(19) United States
(12) Patent Application Publication
(10) Pub. No.: US 2015/0232913 A1
(43) Pub. Date: Aug. 20, 2015

(54) BIOSENSING SYSTEMS AND METHODS FOR ASSESSING ANALYTE CONCENTRATIONS

(71) Applicants: Kenneth F. Reardon, Fort Collins, CO (US); Brian Heinze, Fort Collins, CO (US); Jonathan Vickers, Littleton, CO (US); Kyle Smith, Broomfield, CO (US); Xingfeng Huang, Fort Collins, CO (US); Lauren Magliozzi, Fort Collins, CO (US); Zachary Menard, Fort Collins, CO (US); Devon Osbourne, Fort Collins, CO (US)

(72) Inventors: Kenneth F. Reardon, Fort Collins, CO (US); Brian Heinze, Fort Collins, CO (US); Jonathan Vickers, Littleton, CO (US); Kyle Smith, Broomfield, CO (US); Xingfeng Huang, Fort Collins, CO (US); Lauren Magliozzi, Fort Collins, CO (US); Zachary Menard, Fort Collins, CO (US); Devon Osbourne, Fort Collins, CO (US)

(73) Assignee: OptiEnz Sensors, LLC, Fort Collins, CO (US)

(21) Appl. No.: 14/701,333

(22) Filed: Apr. 30, 2015

Related U.S. Application Data
(63) Continuation-in-part of application No. 13/988,723, filed on May 21, 2013.
(60) Provisional application No. 62/031,333, filed on Jul. 31, 2014.
(60) Provisional application No. 61/510,382, filed on Jul. 21, 2011.

(51) Int. Cl. C12Q 1/32; C12Q 1/54

(52) U.S. Cl. CPC C12Q 1/32; C12Q 1/54

(57) ABSTRACT

The present disclosure relates generally to the materials and methods involving biosensing systems. More specifically, the present disclosure relates to biosensors for making real-time, continuous, and quantitative assessments of analyte concentrations in aqueous environments using oxidases. The present disclosure addresses the need for improved methods and systems for making quantitative assessments of various analytes in a continuous and real-time manner, without the need for expensive and time-consuming laboratory processing.

### RELATED APPLICATIONS

This application is a continuation-in-part application of U.S. patent application Ser. No. 13/988,723, filed on May 21, 2013, which is a national phase application, filed pursuant to 35 U.S.C. §371, which claims the benefit of PCT Patent Application No. PCT/US2011/061956, filed Nov. 22, 2011, which claims the benefit of U.S. Provisional Patent Application Ser. Nos. 61/415,920, filed Nov. 22, 2010, and 61/510,382, filed Jul. 21, 2011. This application also claims priority to U.S. Provisional Patent Application Ser. Nos. 61/986,720, filed Apr. 30, 2014, and 62/031,333, filed Jul. 31, 2014. These applications are incorporated herein in their entirety for all purposes.

### GOVERNMENT RIGHTS

This invention was made with Government support under BES-0529048 and IIP-1345146 awarded by the National Science Foundation. The U.S. Government has certain rights in this invention.

### FIELD

The present disclosure relates generally to the materials and methods involving biosensing systems. More specifically, the present disclosure relates to biosensors comprising oxidases for making real-time, continuous and quantitative assessments of analyte concentrations in an aqueous environment.

### BACKGROUND

The demand for tools that measure chemical components in solution quickly, accurately, and efficiently has increased dramatically in recent years. Industries such as oil and gas exploration, extraction and refinement, food and beverage production, and environmental remediation all require precise measurements of chemical compounds and contaminants. Historically, identifying various compounds in aqueous environments has required physical sample collection, chemical pretreatment and extensive laboratory processing (e.g., gas chromatography) in order to generate a single measurement. Often times, samples are shipped to external laboratories for analysis, with results typically being returned weeks later. Although accurate results may be obtained through this process, it is a complex, time-intensive effort that requires expensive equipment and highly skilled labor.

Given the demand for enhanced techniques for measuring and monitoring various chemical components in a continuous and real-time manner, chemical sensors have been developed for laboratory analysis, industrial process control, physiological assessment, and environmental monitoring. Typically, chemical recognition takes place, followed by the conversion of chemical information into an electrical or optical signal. The basic principles of operation of the chemical sensors utilizing electrochemical and optical transduction are well understood. However, this technology has not yet developed to the point of being compatible with continuous, real-time monitoring systems.

In addition, alcohols such as methanol, ethanol, and butanol are produced and used in large quantities around the world. In the production of these alcohols, continuous monitoring at various points in the synthesis and purification processes would enable on-line monitoring, control, and optimization of those processes. For example, various alcohols are produced by microorganisms (bacteria, yeast, cyanobacteria and the like) and can be used as biofuels and commodity chemicals. Continuous monitoring of alcohols, such as ethanol, methanol, and butanol during these batch or continuous fermentation processes would allow the plant operators to know whether the process was proceeding properly or whether a process upset, such as biological contamination, had occurred. An example in which the continuous monitoring of methanol would be advantageous includes when methanol is a component in the fluids injected into shale formations during hydrofracturing. Flowback water containing methanol must often be treated, and the efficiency and effectiveness of the treatment process can be improved by continuous, on-line monitoring of methanol concentrations.

Additionally, many different products in the daily diets of humans and animals include various forms of carbohydrates (e.g., lactose, sucrose, glucose, galactose, xylose, and the like). For example, the primary sugar in milk is lactose. Lactose is found at levels of about 2-8 percent in milk. Lactose is a disaccharide sugar composed of galactose and glucose. In the dairy industry it is often necessary to measure lactose concentrations. For example, the production of lactose-free milk requires analysis of the lactose levels in the final product and could be optimized by lactose measurements during the process.

Analytical methods that are currently used to measure the concentration of lactose in milk take samples from the milk solutions and then send them to laboratories where they are analyzed. When the milk samples are removed, and during the time it takes to test these samples, the chemistry of the sample often changes and thus the test results may be inaccurate or inconsistent. Current analytical methods have a limited range of measurement because the response of their detection element saturates, returning the same signal for two different concentrations. In order to obtain an accurate concentration measurement under saturating conditions, the solution is diluted and then measured again. This can lead to measurement errors and is not readily suitable for continuous and in-situ measurements.

### SUMMARY

These and other needs are addressed by the various aspects, embodiments, and configurations of the present disclosure.

The present instrumentalities advance the art and overcome the problems discussed above by providing biosensing systems, biosensing elements and methods for use in detecting one or more analytes such as lactose and hydrogen peroxide in milk, milk byproducts, or other solutions that may contain carbohydrate and/or hydrogen peroxide and also providing biosensing systems that allow measurements at high concentrations of an analyte and avoid sample dilution.

In one aspect, a biosensing system that measures lactose concentration in a solution is disclosed, wherein the biosensing system comprises an optode comprising an optical fiber having a first tip (also referred to as the distal tip), and a second tip (also referred to as the proximal end), the first tip is covered by a luminescent transducer layer, the luminescent transducer layer is covered by a biocomponent layer, the biocomponent layer is covered by a porous membrane, the second tip is coupled to a photon-detection device, and the photon-detection device is coupled to a signal processing system.

In one embodiment, the biosensing system inter-relates the lactose concentration in the solution, the depth of the biocomponent layer, the depth of the porous membrane, the diffusion coefficient of the porous membrane, the Kₘ and Vₘₐₓ of the reaction between the biocomponent and lactose are selected such that Da is greater than the value of 1-β and the quotient between Da² and 4β is from about 10 to at least 1000, and Vₘₐₓ is the maximum reaction rate achieved by the biocomponent layer under saturation lactose concentrations, and Kₘ is the lactose concentration at which the reaction rate achieved by the biocomponent layer is half of Vₘₐₓ, and β is the lactose concentration in the said solution divided by Kₘ of said biocomponent for lactose, and hₑ is the thickness of the enzyme biocomponent layer which is embedded within a matrix, and hₚ is the thickness of a porous polymeric or ceramic material which sits atop the enzyme biocomponent layer, and Dₚ is the diffusion coefficient of the polymer coating, and Da is a dimensionless number, and Da is (hₑVₘₐₓhₚ)/(DₚKₘ).

In another embodiment, the biosensing system luminescent transducer layer contains a luminescent agent that is selected from the group consisting of a fluorescent agent, a phosphorescent agent, a bioluminescent agent, or a chemiluminescent agent.

In one embodiment, the biosensing system luminescent transducer layer contains a luminescent agent that is selected from the group consisting of trisodium 8-hydroxy-1,3,6-trisulphonate, fluoro(8-anilino-1-naphthalene sulphonate), tris(bipyridine)ruthenium(II) complex, RuDPP, ruthenium complexes, platinum tetrakis(pentafuorophenyl)porphyrin, platinum complexes, and acridinium- and quinidinium-based reagents, fluorescein, fluoresceinamine, or a fluorescein containing compound.

In one embodiment, the biosensing system biocomponent layer comprises a biocomponent selected from the group consisting of at least one enzyme selected from the group consisting of enzymes from Enzyme Commission numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23.

In one embodiment, the biosensing system biocomponent layer comprises a biocomponent displaced within a matrix comprising a hydrogel or other polymer, and wherein the hydrogel is selected from the group consisting of algal polysaccharides, agarose, alginate, gelatin, collagen, pectin, poly(carbamoyl) sulfonate, locust bean gum, and gellan, and wherein the other polymer is selected from the group consisting of polyacrylamide, polystyrene, polymethacrylate, polyvinylalcohol and polyurethane, and wherein the biocomponent is adsorbed or immobilized within said matrix layer by physisorption or chemisorption.

In one embodiment, the biosensing system biocomponent is bound to the matrix layer through adding crosslinking agents to the biocomponent disposed within the matrix layer, and wherein the crosslinking agents are selected from the group consisting of glutaraldehyde, hexamethylene diisocyanate and 1,5-dinitro-2,4-difluorobenzene, glutaraldehyde, polyethyleneimine, hexamethylenediamine and formaldehyde.

In one embodiment, the biosensor luminescent transducer layer is bound in a layer of molecules bound to the first tip of the optical fiber, the layer of molecules is selected from the group consisting of cellulose, cellulose derivatives, silica, glass, dextran, starch, agarose, porous silica, chitin and chitosan.

In one embodiment, the biosensing system has a membrane that is polycarbonate having a pore size of from about 0.005 μm to about 0.025 μm.

In one embodiment, the biosensing system has a membrane that comprises a coating of polyurethane.

In one embodiment, the biosensing system biocomponent is beta-galactosidase and glucose oxidase and wherein said luminescent transducer layer interacts with oxygen.

In one embodiment, the biosensing system biocomponent is beta-galactosidase, glucose oxidase and catalase and wherein said luminescent transducer layer interacts with oxygen.

In one embodiment, the biosensing system biocomponent is beta-galactosidase and glucose oxidase and wherein said luminescent transducer layer interacts with protons.

In one embodiment, the biosensing system biocomponent is beta-galactosidase, glucose oxidase and catalase and wherein said luminescent transducer layer interacts with protons.

In one embodiment, the biosensing system biocomponent is beta-galactosidase and glucose oxidase and wherein said luminescent transducer layer interacts with oxygen and protons.

In one embodiment, the biosensing system biocomponent is beta-galactosidase, glucose oxidase and catalase and wherein said luminescent transducer layer interacts with oxygen and protons.

In one embodiment, the biosensing system biocomponent is beta-galactosidase and galactose oxidase and wherein said luminescent transducer layer interacts with oxygen.

In one embodiment, the biosensing system biocomponent is beta-galactosidase, galactose oxidase and catalase and wherein said luminescent transducer layer interacts with oxygen.

In one embodiment, the biosensing system biocomponent is beta-galactosidase and galactose oxidase and wherein said luminescent transducer layer interacts with protons.

In one embodiment, the biosensing system biocomponent is beta-galactosidase, galactose oxidase and catalase and wherein said luminescent transducer layer interacts with protons.

In one embodiment, the biosensing system biocomponent is beta-galactosidase and galactose oxidase and wherein said luminescent transducer layer interacts with oxygen and protons.

In one embodiment, the biosensing system biocomponent is beta-galactosidase, galactose oxidase and catalase and wherein said luminescent transducer layer interacts with oxygen and protons.

In one embodiment, the biosensing system biocomponent is carbohydrate oxidase and wherein said luminescent transducer layer interacts with oxygen.

In one embodiment, the biosensing system biocomponent is carbohydrate oxidase and catalase and wherein said luminescent transducer layer interacts with oxygen.

In one embodiment, the biosensing system biocomponent is carbohydrate oxidase and wherein said luminescent transducer layer interacts with protons.

In one embodiment, the biosensing system biocomponent is carbohydrate oxidase and catalase and wherein said luminescent transducer layer interacts with protons.

In one embodiment, the biosensing system biocomponent is carbohydrate oxidase and wherein said luminescent transducer layer interacts with oxygen and protons.

In one embodiment, the biosensing system biocomponent is carbohydrate oxidase and catalase and wherein said luminescent transducer layer interacts with oxygen and protons.

In one embodiment, the biosensing system biocomponent is cellobiose dehydrogenase and wherein said luminescent transducer layer interacts with protons.

In one aspect, a method of measuring the concentration of lactose in a solution is disclosed, the method comprises, communicating the interaction of a biocomponent with the lactose in the solution to a display and/or data storage device by communication means, the communication means comprising said biocomponent, lactose, oxygen and/or protons, a porous membrane, a biocomponent layer, a transducer layer, an optical fiber, a photon-detection device, a signal processor and said display and/or data storage device, the porous member separates the biocomponent layer from the solution, the biocomponent layer comprises the biocomponent displaced within a matrix, the biocomponent interacts with the lactose and either uses or generates oxygen and/or protons in the solution during the interaction, and the biocomponent layer is in contact with the transducer layer, and the transducer layer luminesces and wherein the luminescence is partially quenched by the oxygen and/or protons, and the luminescence is communicated to the photon-detection device through said optical fiber having a first end and a second end, the first end of the optical fiber is in contact and communicates with the transducer layer and the aid second end of the optical fiber is in contact and communicates with the signal processor, and the signal processor processes the communication from the luminescence of the transducer layer into a communication comprising the concentration of lactose in the solution, and the signal processor communicates the concentration of lactose in the solution to the display and/or data storage device.

In one embodiment, the method of measuring lactose concentration in the solution uses the following variables and the following algorithm in order to construct a biosensing system that measures lactose in the linear response range, the variables are the concentration of lactose in the solution, the depth of the biocomponent layer, the depth of the porous membrane, the diffusion coefficient of the porous membrane, the Kₘ and Vₘₐₓ of the reaction between the biocomponent and lactose are selected such that Da is greater than the value of 1-β and the quotient between Da² and 4β is from about 10 to at least 1000, and wherein Vₘₐₓ is the maximum reaction rate achieved by the biocomponent layer under saturation lactose concentrations, and wherein Kₘ is the lactose concentration at which the reaction rate achieved by the biocomponent layer is half of Vₘₐₓ, and wherein β is the lactose concentration in the said solution divided by Kₘ of said biocomponent for lactose, and wherein hₑ is the thickness of the enzyme biocomponent layer which is embedded within a matrix, and wherein hₚ is the thickness of a porous polymeric or ceramic material which sits atop the enzyme biocomponent layer, and wherein Dₚ is the diffusion coefficient of the polymer coating, and wherein Da is a dimensionless number, and wherein Da is (hₑVₘₐₓhₚ)/(DₚKₘ).

In one aspect, a biosensing system that detects carbohydrates in a solution is disclosed wherein the biosensing system comprises a biocomponent and a transducer. In one embodiment, the biosensing system has a biocomponent that is selected from the group consisting of enzymes from Enzyme Commission numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23. In one embodiment, the biosensing system has a biocomponent that is catalase and at least one enzyme selected from the group consisting of Enzyme Commission numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23. In one embodiment, the biosensing system has a transducer that interacts with oxygen. In one embodiment, the biosensing system has a transducer that that interacts with protons. In one embodiment, the biosensing system has a transducer that interacts with oxygen and protons.

In one aspect, a biosensing system that detects carbohydrate in a solution is disclosed wherein the biosensing system comprises a biocomponent, and a transducer, and a photon-detection device, and a signal processing system. In one embodiment, the biosensing system has a biocomponent that is selected from the group consisting of enzymes from Enzyme Commission numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23. In one embodiment, the biosensing system has a biocomponent that is catalase and at least one enzyme selected from the group consisting of Enzyme Commission numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23. In one embodiment, the biosensing system has a transducer that interacts with oxygen. In one embodiment, the biosensing system has a transducer that interacts with protons. In one embodiment, the biosensing system has a transducer that interacts with oxygen and protons.

In an aspect, a biosensing system that detects lactose in a solution is disclosed wherein the biosensing system comprises a biocomponent, and a transducer, and a photon-detection device, and a signal processing system. In an embodiment, the biosensing system biocomponent is beta-galactosidase and glucose oxidase and the transducer interacts with oxygen. In an embodiment, the biosensing system biocomponent is beta-galactosidase, glucose oxidase and catalase and the transducer interacts with oxygen. In an embodiment, the biosensing system biocomponent is beta-galactosidase and glucose oxidase and the transducer interacts with protons. In an embodiment, the biosensing system biocomponent is beta-galactosidase, glucose oxidase and catalase and the transducer interacts with protons. In an embodiment, the biosensing system biocomponent is beta-galactosidase and glucose oxidase and the transducer interacts with oxygen and protons. In an embodiment, the biosensing system biocomponent is beta-galactosidase, glucose oxidase and catalase and the transducer interacts with oxygen and protons. In an embodiment, the biosensing system biocomponent is beta-galactosidase and galactose oxidase and the transducer interacts with oxygen. In an embodiment, the biosensing system biocomponent is beta-galactosidase, galactose oxidase and catalase and the transducer interacts with oxygen. In an embodiment, the biosensing system biocomponent is beta-galactosidase and galactose oxidase and the transducer interacts with protons. In an embodiment, the biosensing system biocomponent is beta-galactosidase, galactose oxidase and catalase and the transducer interacts with protons. In an embodiment, the biosensing system biocomponent is beta-galactosidase and galactose oxidase and the transducer interacts with oxygen and protons. In an embodiment, the biosensing system biocomponent is beta-galactosidase, galactose oxidase and catalase and the transducer interacts with oxygen and protons. In an embodiment, the biosensing system biocomponent is carbohydrate oxidase and the transducer interacts with oxygen. In an embodiment, the biosensing system biocomponent is carbohydrate oxidase and catalase and the transducer interacts with oxygen. In an embodiment, the biosensing system biocomponent is carbohydrate oxidase and the transducer interacts with protons. In an embodiment, the biosensing system biocomponent is carbohydrate oxidase and catalase and the transducer interacts with protons. In an embodiment, the biosensing system biocomponent is carbohydrate oxidase and the transducer interacts with oxygen and protons. In an embodiment, the biosensing system biocomponent is carbohydrate oxidase and catalase and the transducer interacts with oxygen and protons. In an embodiment, the biosensing system biocomponent is cellobiose dehydrogenase and the transducer interacts with protons.

In one aspect, a biosensing system that measures hydrogen peroxide in a solution is disclosed wherein the biosensing element comprises a biocomponent and a transducer. In an embodiment, the biosensing system biocomponent is catalase and the transducer interacts with oxygen.

In an aspect, a biosensing system that detects hydrogen peroxide in a solution is disclosed wherein the biosensing system comprises a biocomponent, and a transducer, and a photon-detection device, and a signal processing system. In an embodiment, the biosensing system biocomponent is catalase and the transducer interacts with oxygen.

In an aspect, a biosensing system that detects lactose and hydrogen peroxide in a solution is disclosed wherein the biosensing system comprises a biocomponent, and a transducer, and a photon-detection device, and a signal processing system. In one embodiment, the biosensing system biocomponent is cellobiose dehydrogenase and catalase and the transducer layer interacts with oxygen and protons.

In one aspect, a method of detecting carbohydrate in a solution involves placing a biosensing system into contact with a solution containing carbohydrate, wherein the biosensing system comprises a biocomponent that interacts with the carbohydrate to consume oxygen, and the biocomponent is in contact with a transducer that luminesces and whose luminescence is partially quenched with oxygen, and the transducer is in contact with an optical fiber or other optical device that transfers photons to a photon-detection device that thereby transfers the luminescent photons of the transducer to a photon detection device and a signal processing system that provides the value of the concentration of the carbohydrate in the solution. In one embodiment, the method uses a biocomponent comprising catalase and at least one enzyme selected from the group consisting of Enzyme Commission numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23. In another embodiment, the method uses a transducer that comprises a RuDPP-based oxygen optode. In one embodiment, the method uses a photon-detecting device that comprises an image sensor and a signal processing system that comprises a transimpedance amplifier whose output is coupled to a microprocessor whose output is coupled to a display that displays the concentration of the carbohydrate in the solution. In one embodiment, the biosensing element of a biosensing system that detects lactose and hydrogen peroxide in a solution comprises a first biocomponent that reacts with lactose and a second biocomponent that reacts with hydrogen peroxide, wherein the first biocomponent is one or more enzymes selected from the group consisting of beta-galactosidase, glucose oxidase, galactose oxidase, cellobiose dehydrogenase and carbohydrate oxidase, and wherein the second biocomponent is catalase, and wherein the first biocomponent and the second biocomponent are within the same cells, and wherein the cells are immobilized within a matrix, and wherein the matrix is in contact with a transducer layer. In one embodiment, the cells are alive. In one embodiment, the cells are dead. In one embodiment, the transducer layer is comprised of a first chemical transducer that interacts with oxygen and a second chemical transducer that interacts with protons.

In one aspect, the sensing element of a biosensing system that detects lactose and hydrogen peroxide in a solution is disclosed wherein the sensing element comprises a first biocomponent that reacts with lactose and a second biocomponent that reacts with hydrogen peroxide, and the first biocomponent is one or more enzymes selected from a group consisting of beta-galactosidase, glucose oxidase, galactose oxidase, cellobiose dehydrogenase and carbohydrate oxidase, and wherein the second biocomponent is catalase, and wherein the first biocomponent and the second biocomponent are immobilized within a matrix, and wherein the matrix is in contact with a transducer layer.

In one aspect, a method for detecting the concentration of lactose and hydrogen peroxide in a solution is disclosed wherein a first biosensing system detects the lactose concentration and a second biosensing system detects the hydrogen peroxide concentration.

Embodiments of the present disclosure include biosensing systems for measuring the concentration of an analyte in a solution. The biosensing systems include an optode comprising an optical fiber having a distal tip and a proximal tip, a photon-detection device coupled to the proximal tip, and a signal processing system coupled to the photon-detection device. The distal tip includes a transducer layer and a biocomponent layer, and the biocomponent layer further includes at least one oxidase from Enzyme Commission number 1 (EC 1) that catalyzes a chemical reaction with the analyte. The transducer layer converts an input signal generated from the chemical reaction with the analyte in the biocomponent layer into an output signal detectable by the photon-detection device, and the signal processing system generates a value from the output signal detectable by the photon-detection device that corresponds to the concentration of the analyte in the solution.

In one embodiment, the transducer layer further includes one or more of a fluorescent luminescent agent, a phosphorescent luminescent agent, a bioluminescent luminescent agent, a chemiluminescent luminescent agent, and derivatives and combinations thereof. In some embodiments, the transducer layer includes one or more of trisodium 8-hydroxy-1,3,6-trisulphonate, fluoro (8-anilino-1-naphthalene sulphonate), tris(bipyridine)ruthenium(II) complex, RuDPP, ruthenium complexes, platinum tetrakis(pentafuorophenyl)porphyrin, platinum complexes, acridinium-based reagents, quinidinium-based reagents, fluorescein, fluoresceinamine, a fluorescein containing compound, and derivatives and combinations thereof.

In one embodiment, the biosensing system includes at least one oxidase categorized in EC numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, 3.2.1.23, 1.1.3.2, 1.1.3.4, 1.1.3.10, 1.1.3.13, and derivatives and combinations thereof. In some embodiments, the biocomponent layer includes a hydrogel matrix having one or more of algal polysaccharides, agarose, alginate, gelatin, collagen, pectin, poly(carbamoyl)sulfonate, locust bean gum, gellan, and combinations and derivatives thereof. In some embodiments, the biocomponent layer includes a matrix having a cross-linking agent and one or more of a bovine serum albumin, a lysozyme, alginate, a sol-gel polyvinyl alcohol, and combinations and derivatives thereof. In some embodiments, the cross-linking agent is one or more of glutaraldehyde, hexamethylene diisocyanate and 1,5-dinitro-2,4-difluorobenzene, polyethyleneimine, hexamethylenediamine formaldehyde, and combinations and derivatives thereof. In other embodiments, the biocomponent layer includes a matrix that is neither hydrogel-based nor a cross-linked polymer, but is a sol-gel-based matrix.

In one embodiment, the biosensing system includes a distal tip that further includes one or more polymer-based diffusion layers. In some aspects, the biosensing the one or more polymer-based diffusion layers include one or more of a polyurethane-based polymer and a tetrafluoroethylene-based fluoropolymer, and combinations and derivatives thereof.

In one embodiment, the biocomponent layer further includes one or more enzymes categorized in EC numbers 1.11.1, 1.11.1.6, 1.11.1.7, and combinations and derivatives thereof.

In one embodiment, the biocomponent layer further includes one or more stabilizing agents, including but not limited to, one or more of β-mercaptoethanol, cysteine, dithitreitol (DTT) α-thioglycerol, and other thiol containing reducing agents and combinations and derivatives thereof.

In one embodiment, the biosensing system is used to detect and/or quantify an analyte that is a carbohydrate, including but not limited to, glucose, galactose, sucrose, and xylose. In another embodiment, the biosensing system is used to detect and/or quantify an analyte that is an alcohol, including but not limited to ethanol, methanol, or butanol. In another embodiment, the biosensing system uses at least one oxidase to detect and/or quantify an analyte, including oxidases categorized in EC numbers 1.1.3.2, 1.1.3.4, 1.1.3.10 and 1.1.3.13. In some aspects, the oxidase is a purified and/or isolated enzyme.

Embodiments of the present disclosure include a method for measuring the concentration of an analyte in a solution. The method includes obtaining a biosensing system having an optode that includes an optical fiber having a distal tip and a proximal tip, a photon-detection device coupled to the proximal tip, and a signal processing system coupled to the photon-detection device; placing the distal tip into the solution, the distal tip comprising a transducer layer and a biocomponent layer, wherein the biocomponent layer comprises at least one oxidase from Enzyme Commission number 1 (EC 1) that catalyzes a chemical reaction with the analyte, wherein the transducer layer converts an input signal generated from the chemical reaction with the analyte in the biocomponent layer into an output signal detectable by the photon-detection device; and using the signal processing system to generate a value from the output signal detectable by the photon-detection device that corresponds to the concentration of the analyte in the solution.

In one embodiment, the method further includes sterilizing the distal tip of the biosensing system prior to use. In another embodiment, the method includes using the value from the output signal to assess one or more bioprocesses of a microorganism.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1. Standard curve generated from hydrogen peroxide standards measured using a biosensing system, according to one embodiment of the present disclosure. Signal change was measured relative to a blank solution of phosphate buffer having a pH of 7.2.

FIG. 2. Standard curve generated from lactose standards measured using a biosensing system, according to one embodiment of the present disclosure. Signal change was measured relative to a blank solution of phosphate buffered saline (pH 7.4).

FIG. 3. Response curve generated from lactose standards measured using a lactose biosensing system after 0 and 48 hours in solution at pH 4.8 and 40° C., according to one embodiment of the present disclosure. Signal change was measured relative to a blank solution containing no lactose.

FIG. 4. Response curve generated from H₂O₂ standards measured using a peroxide biosensing system after 0 and 19 hours in solution at pH 4.8 and 40° C., according to one embodiment of the present disclosure. Signal change was measured relative to a blank solution containing no H₂O₂.

FIG. 5. Response curve generated from lactose standards measured using a lactose biosensing system after 0 and 16 h in solution at pH 6.5 and temperature 49° C., according to one embodiment of the present disclosure. Signal change was measured relative to a blank solution containing no lactose.

FIG. 6. Response curve generated from H₂O₂ standards measured using a peroxide biosensing system after 0 and 16 h in solution at pH 6.5 and temperature 49° C., according to one embodiment of the present disclosure. Signal change was measured relative to a blank solution containing no H₂O₂.

FIG. 7. Graphical representation of Michaelis-Menten equation relationships between enzyme reaction rate and substrate concentration. Kₘ is the concentration of substrate at which the reaction rate is equal to ½ the reaction rate under saturating substrate conditions (Vₘₐₓ) of the enzymatic reaction.

FIG. 8. Representation of enzymatic biosensing element for measuring analytes in high concentrations, according to one embodiment of the present disclosure. D_{b} is the diffusion coefficient of a substrate/analyte in the bulk solution; D_{b} is the diffusion coefficient of a substrate/analyte in the polymer/ceramic coating; Dₑ is the diffusion coefficient of a substrate/analyte in the enzyme layer which is embedded within a matrix; hₚ is the height of the polymer/ceramic coating; and hₑ is the height of the enzyme layer which is embedded within a matrix. The enzyme layer sits atop a transducer which may be part of an optode.

FIG. 9. Response curve for a lactose biosensing system that includes a lactose sensor with a thin film of enzyme immobilized on the surface, according to one embodiment of the present disclosure. Signal change was measured relative to a blank solution of phosphate buffered saline (pH 7.4).

FIG. 10. Response curve for a lactose biosensing system that includes a lactose sensor with a porous diffusion layer, according to one embodiment of the present disclosure. Signal change was measured relative to a blank solution of phosphate buffered saline (pH 7.4).

FIG. 11. Response curve for a lactose biosensing system that includes a lactose sensor having a less porous diffusion layer compared to the porous diffusion layer used in the lactose biosensing system of FIG. 10, according to one embodiment of the present disclosure. Signal change was measured relative to a blank solution of phosphate buffered saline (pH 7.4).

FIG. 12. System for providing design parameters used for constructing biosensing elements, according to one embodiment of the present disclosure.

FIG. 13. Schematic representation of a biosensing system, according to one embodiment of the present disclosure.

FIG. 14. Schematic representation of the distal tip (i.e., first tip) of a biosensing system, according to one embodiment of the present disclosure.

FIG. 15. Schematic representation of exemplary method for using a biosensing system to measure the concentration of an analyte in a solution, according to one embodiment of the present disclosure.

FIG. 16. Response curve for a glucose biosensing system that includes a glucose sensor having a reduced amount of glucose oxidase immobilized in a cross-linked BSA matrix, according to one embodiment of the present disclosure.

FIG. 17. Response curves for glucose biosensing systems that include thick or thin polyurethane diffusion layer and a glucose sensor having glucose oxidase immobilized in a cross-linked BSA matrix, according to one embodiment of the present disclosure.

FIG. 18. Response curve for a glucose biosensing system that includes a glucose sensor having glucose oxidase immobilized in a cross-linked lysozyme polymer matrix, according to one embodiment of the present disclosure.

FIG. 19. Response curve for a glucose biosensing system that includes a glucose sensor having glucose oxidase immobilized in an alginate polymer matrix, according to one embodiment of the present disclosure.

FIG. 20. Response curves for glucose biosensing systems that include glucose sensors having glucose oxidase and catalase either mixed in a single layer or in separate layers, according to one embodiment of the present disclosure.

FIG. 21. Response curve for a glucose biosensing system that includes a glucose sensor having glucose oxidase immobilized in a sol-gel polymer matrix, according to one embodiment of the present disclosure.

FIG. 22. Response curve for a glucose biosensing system that includes a glucose sensor having lactose oxidase immobilized in a cross-linked BSA polymer, according to one embodiment of the present disclosure.

FIGS. 23A-23B. Response curves for glucose biosensing systems that include glucose sensors having glucose oxidase immobilized in a cross-linked BSA matrix, with (FIG. 23B) and without (FIG. 23A) mercaptoethanol treatment, according to one embodiment of the present disclosure.

FIG. 24. Response curve for a sucrose biosensing system that includes a sucrose sensor having lactose oxidase immobilized in a cross-linked BSA matrix, according to one embodiment of the present disclosure.

FIG. 25. Response curve for a galactose biosensing system that includes a galactose sensor having lactose oxidase immobilized in a cross-linked BSA matrix, according to one embodiment of the present disclosure.

FIG. 26. Response curve for a glucose biosensing system that includes a glucose sensor having pyranose oxidase immobilized in a cross-linked BSA matrix, according to one embodiment of the present disclosure.

FIG. 27. Response curve for a xylose biosensing system that includes a xylose sensor having pyranose oxidase immobilized in a cross-linked BSA matrix, according to one embodiment of the present disclosure.

FIG. 28. Graphical representation demonstrating the effects of pH on glucose concentration measurements taken using glucose biosensing systems, according to one embodiment of the present disclosure.

FIG. 29. Graphical representation of the changes in glucose concentrations during aerobic fermentation of B. atrophaeus determined using a glucose biosensing system having a thick polyurethane diffusion layer, according to one embodiment of the present disclosure.

FIG. 30. Graphical representation of the changes in glucose concentrations during aerobic fermentation of P. stipitis determined using a glucose biosensing system having a thick polyurethane diffusion layer, according to one embodiment of the present disclosure.

FIG. 31. Response curves comparing the effects of sterilization using gamma irradiation on the activity of glucose biosensing systems, according to one embodiment of the present disclosure.

FIG. 32. Response curves for two glucose biosensing systems sterilized with a chemical sterilization agent, according to one embodiment of the present disclosure.

FIG. 33. Response curves comparing the effects of temperature incubation at 50° C. on the activity of glucose biosensing systems having glucose oxidase from Aspergillis niger (Biomatik, Cat. No. A4149) immobilized in a cross-linked BSA matrix, according to one embodiment of the present disclosure.

FIG. 34. Response curves comparing the effects of temperature incubation at 50° C. on the activity of glucose biosensing systems having glucose oxidase from Aspergillis niger (Sigma Aldrich, Cat. No. G2133) immobilized in a cross-linked BSA matrix, according to one embodiment of the present disclosure.

FIG. 35. Response curves comparing the effects of temperature incubation at 50° C. on the activity of glucose biosensing systems having glucose oxidase from Aspergillis niger (EMD/Calbiochem, Cat. No. 345386) immobilized in a cross-linked BSA polymer matrix, according to one embodiment of the present disclosure.

FIG. 36. Response curves comparing the effects of temperature incubation at 50° C. on the activity of glucose biosensing systems having glucose oxidase from Aspergillis niger (Sigma Aldrich, Cat. No. G6125) immobilized in a cross-linked BSA matrix, according to one embodiment of the present disclosure.

FIG. 37. Response curve for an ethanol biosensing system that includes an ethanol sensor having alcohol oxidase immobilized in a cross-linked BSA matrix, according to one embodiment of the present disclosure.

FIG. 38. Response curve for a butanol biosensing system that includes a butanol sensor having alcohol oxidase immobilized in a cross-linked BSA matrix, according to one embodiment of the present disclosure.

FIG. 39. Response curve for a methanol biosensing system that includes a methanol sensor having alcohol oxidase immobilized in a cross-linked BSA matrix, according to one embodiment of the present disclosure.

FIG. 40. Response curve for a methanol biosensing system that includes a methanol sensor having alcohol oxidase immobilized in a sol gel-polyvinyl alcohol polymer matrix, according to one embodiment of the present disclosure.

FIG. 41. Response curve for a methanol biosensing system that includes a methanol sensor having a Nafion coating, according to one embodiment of the disclosure.

FIG. 42. Response curve of the continuous sensing of methanol in a flow-through chamber, according to one embodiment of the present disclosure.

FIG. 43. Graphical representation comparing the active lifetime of methanol biosensing systems with and without catalase, according to one embodiment of the present disclosure.

### DETAILED DESCRIPTION

The present disclosure relates generally to the materials and methods involving biosensing systems. More specifically, the present disclosure relates to biosensors comprising oxidases for making real-time, continuous and quantitative assessments of analyte concentrations in an aqueous environment.

One way to provide in-line analysis in a sample is to use a biosensing system. Biosensing systems offer the potential of measurements that are specific, continuous, rapid, and reagentless. Biosensing elements of biosensing systems combine a biocomponent which is coupled to a transducer to yield a device capable of measuring chemical concentrations. A biocomponent may be any biological detection agent. Examples of biocomponents include enzymes, whole cells, microorganisms, RNA, DNA, aptamers and antibodies. The biocomponent interacts with an analyte via a binding event and/or reaction. The role of the transducer is to convert the biocomponent detection event into a signal, usually optical or electrical. A transducer is typically a physical sensor such as an electrode, or a chemical sensor. The analyte normally interacts with the biocomponent through a chemical reaction or physical binding. For example, in the case of a biosensing system that uses an enzyme biocomponent, the enzyme biocomponent would react with the analyte of interest and a product or reactant of the enzyme catalyzed reaction such as oxygen, ammonia, hydrochloric acid or carbon dioxide, may be detected by an optical, electrochemical or other type of transducer.

### DEFINITIONS

Biocomponent: A biocomponent binds, catalyzes a reaction of, or otherwise interacts with analytes, compounds, atoms or molecules. A biocomponent may refer to a single type or species of biocomponent or may refer to a mixture of multiple types or species of biocomponent. A biocomponent may alternatively be referred to in the plural form as biocomponents. Biocomponents may refer to multiple singular species of biocomponents or to multiple different types of species of biocomponents. Non-limiting examples of biocomponents include aptamers, DNA, RNA, proteins, enzymes, antibodies, cells, whole cells, tissues, single-celled microorganisms, and multicellular microorganisms. A biocomponent may be a cell or microorganism that has biocomponent enzymes within the cell or microorganism.

Analyte: An analyte is the substance or chemical constituent that is to be measured. In a reaction based biosensing system, the reaction of the analyte with a biocomponent causes a change in the concentration of a reactant or product that is measurable by the transducer. An analyte may also be a substrate of an enzyme. In other biosensing systems, the biocomponent may bind the analyte and not catalyze a reaction.

Transducer: A transducer is a device or compound which converts an input signal into an output signal of a different form. A transducer may convert a chemical input signal into an optical output signal, for example. A transducer may also be a device or compound that receives energy from one system and supplies energy of either the same or of a different kind to another system, in such a manner that the desired characteristics of the energy input appear at the output. In a reaction-based biosensing system, a transducer is a substance or device that interacts with the atoms, compounds, or molecules produced or used by the biocomponent. The interaction of the transducer with the atoms, compounds, or molecules produced or used by the biocomponent causes a signal to be generated by the transducer. The transducer may also generate a signal as an inherent property of the transducer. The signal may be an electrical current, a photon, a luminescence, or a switch in a physical configuration. In one embodiment, the signal produced by the transducer is quenched by a reactant or product of the biocomponent.

Optical transducer: An optical transducer is an optode that incorporates a luminescent reagent that luminesces. The luminescent reagent interacts with an atom, molecule, or compound and that interaction causes a change in the intensity and/or lifetime of the fluorescence of the optical transducer.

Physical transducer: A physical transducer is a device that interacts with an atom, molecule, photon or compound and that interaction causes a shift in its physical properties.

Biosensor: A biosensor measures compounds, atoms or molecules using a biocomponent. A biosensor may alternatively be referred to as a biosensing system and/or a biosensing element.

Biosensing system: A biosensing system contains a biosensing element, a photon-detection device, and a signal processing system. A biosensing system may alternatively be referred to as a biosensor system. Biosensing system may alternatively refer to various parts of the biosensing system such as the biosensing element, for example.

Biosensing element: A biosensing element detects analytes. A biosensing element comprises a biocomponent and a transducer. In certain embodiments, a biosensing element comprises a biocomponent, a transducer and/or an optode.

Crosslinking: Crosslinking is the process of linking polymeric molecules to one another. Crosslinking may be through chemical bonds or ionic interactions.

Matrix: A matrix is an interlacing, repeating cell, net-like or other structure that embodies the biocomponents. The immobilization material is an example of a matrix.

Immobilization material: Immobilization material is the substance, compound or other material used to immobilize the biocomponent onto the biosensing element transducer layer. The immobilization material may be a matrix or may be less ordered than a matrix.

Optode: An optode is an optical sensor device that optically measures a specific substance or quantity. An optode is one type of optical transducer. In one embodiment, for example, an optode requires a luminescent reagent, a polymer to immobilize the luminescent reagent and instrumentation such as a light source, detectors and other electronics. Optodes can apply various optical measurement schemes such as reflection, absorption, an evanescent wave, luminescence (for example fluorescence and phosphorescence), chemiluminescence, and surface plasmon resonance.

pH sensor: A pH sensor measures the concentration of hydrogen ions in a solution.

pH optode: A pH optode is an optode that has a detection element that interacts with hydrogen ions. An example of a detection element that interacts with hydrogen ions is, fluorescein, fluoresceinamine or other fluorescein containing compounds. In an embodiment, for example, a pH optode based on luminescence has a luminescent reagent that is pH responsive.

Luminescence: Luminescence is a general term which describes any process in which energy is emitted from a material at a different wavelength from that at which it is absorbed. Luminescence may be measured by intensity and/or by lifetime decay. Luminescence is an umbrella term covering fluorescence, phosphorescence, bioluminescence, chemoluminescence, electrochemiluminescence, crystalloluminescence, electroluminescence, cathodoluminescence, mechanoluminescence, triboluminescence, fractoluminescence, piezoluminescence, photoluminescence, radioluminescence, sonoluminescence, and thermoluminescence.

Fluorescence: Fluorescence is a luminescence phenomenon in which electron de-excitation occurs almost spontaneously, and in which emission from a luminescent substance ceases when the exciting source is removed. Fluorescence may be measured by intensity and/or by lifetime of the decay.

Phosphorescence: Phosphorescence is a luminescence phenomenon in which light is emitted by an atom or molecule that persists after the exciting source is removed. It is similar to fluorescence, but the species is excited to a metastable state from which a transition to the initial state is forbidden. Emission occurs when thermal energy raises the electron to a state from which it can de-excite. Phosphorescence may be measured by intensity and/or by lifetime of the decay.

Oxygen sensor: An oxygen sensor measures the concentration of oxygen in a solution.

Oxygen optode: An oxygen optode is an optode that has a detection element that interacts with oxygen. An example of a detection element that interacts with oxygen is Tris(4,7-diphenyl-1,10-phenanthroline)Ru(II) chloride, also known as RuDPP. Other examples include, but are not limited to, trisodium 8-hydroxy-1,3,6-trisulphonate, fluoro (8-anilino-1-naphthalene sulphonate), tris(bipyridine)ruthenium(II) complex, ruthenium complexes, platinum tetrakis(pentafuorophenyl)porphyrin, platinum complexes, acridinium-based reagents, quinidinium-based reagents, fluorescein, fluoresceinamine, a fluorescein containing compound, and derivatives and combinations thereof, as would be readily understood by one of ordinary skill in the art based on the present disclosure.

Photon-detection device: A photon-detection device is a class of detectors that multiply the current produced by incident light by as much as 100 million times in multiple dynode stages, enabling, for example, individual photons to be detected when the incident flux of light is very low. Photon-detection devices may be vacuum tubes, solid state photomultipliers or other devices that interact with incident light, and amplify or otherwise process the signal and/or photons produced by that interaction. Alternative embodiments of a photon-detection device include an image sensor, CCD sensors, CMOS sensors, photomultiplier tubes, charge coupled devices, photodiodes and avalanche photodiodes.

Signal processing system: A signal processing system processes the signal from a biosensing system into information that can be displayed to an end user. An example of a signal processing system is a converter or sampler device such as a signal processor or a transimpedance amplifier that accepts the output of a photon-detection device and in turn provides the input of a microprocessor that converts the signal into an output corresponding to the concentration of an analyte within the solution that was measured by the biosensing system. The output of the microprocessor is then communicated to an end user, for example by displaying the concentration on a screen.

Image sensor: An image sensor is a device that converts an optical image to an electric signal. Examples of image sensors include charge-coupled devices (CCD) or complementary metal-oxide-semiconductor (CMOS) active pixel sensors.

Sampler device: A sampler device reduces a continuous signal to a discrete signal. A common example is the conversion of a sound wave or light wave (a continuous signal) to a sequence of samples (a discrete-time signal).

Solution: A mixture of one or more substances in one or more liquids. Solution does not necessarily indicate that a particular substance is a solid dissolved in a liquid, and does not necessarily indicate a particular degree of homogeneity or non-homogeneity. A solution may be aqueous, or water-based, or a solution may be based primarily of a different liquid, such as an alcohol(s). A solution may or may not include microorganisms or other components such as, but not limited to, flavors, sweeteners, growth modifiers, emulsifiers, food colors, acidulants, pH adjusting agents, stabilizers, and the like.

Avalanche photodiode: An avalanche photodiode (APD) is a highly sensitive semiconductor electronic device that exploits the photoelectric effect to convert light to electricity. APDs can be thought of as photodetectors that provide a built-in first stage of gain through avalanche multiplication.

Converter: A converter is a current-to-voltage converter, and is alternatively referred to as a transimpedance amplifier. A converter is an electrical device that takes an electric current as an input signal and produces a corresponding voltage as an output signal. In another embodiment a converter may be a voltage-to-current converter.

Amperometric: Amperometric means to measure an electrical current.

Damkohler numbers (Da): Da are dimensionless numbers used to relate chemical reaction timescales to other phenomena occurring in a system. Da represents a dimensionless reaction time.

Michaelis-Menten equation: The Michaelis-Menten equation describes the rate of enzymatic reactions by relating reaction rate ν to [S], the concentration of a substrate S. Vₘₐₓ is the maximum rate achieved by the system, at maximum (saturating) substrate concentrations. The Michaelis constant Kₘ is the substrate concentration at which the reaction rate is half of Vₘₐₓ. The equation is as follows:

$$v = \frac{{V}_{max} [ S ]}{{K}_{m} + [ S ]}$$

Enzyme Commission number (EC number): The enzyme commission number is a nomenclature system used to classify enzymes by the reactions they catalyze. The recommendations of the Nomenclature Committee of the International Union of Biochemistry and Molecular Biology on the Nomenclature and Classification of Enzymes by the Reactions they Catalyse determine the EC number of an enzyme. For example, EC number 1 corresponds to oxidoreductases (also referred to as dehydrogenases or oxidases). Within the EC 1 category, oxidoreductases can be further classified into 22 subclasses, such as EC 1.11 (or EC 1.11.1), which corresponds to peroxidases. These subclasses can be divided into further classes, such as EC 1.11.1.6, which corresponds to catalase. EC numbers and classification nomenclature would be apparent to one of ordinary skill in the art based on the present disclosure.

EC number 1.1.3: EC number 1.1.3 includes oxidoreductases that act on the CH—OH group of donors with oxygen as an acceptor such as: EC 1.1.3.3 malate oxidase, EC 1.1.3.4 glucose oxidase, EC 1.1.3.5 hexose oxidase, EC 1.1.3.6 cholesterol oxidase, EC 1.1.3.7 aryl-alcohol oxidase, EC 1.1.3.8 L-gulonolactone oxidase, EC 1.1.3.9 galactose oxidase, EC 1.1.3.10 pyranose oxidase, EC 1.1.3.11 L-sorbose oxidase, EC 1.1.3.12 pyridoxine 4-oxidase, EC 1.1.3.13 alcohol oxidase, EC 1.1.3.14 catechol oxidase (dimerizing), EC 1.1.3.15 (S)-2-hydroxy-acid oxidase, EC 1.1.3.16 ecdysone oxidase, EC 1.1.3.17 choline oxidase, EC 1.1.3.18 secondary-alcohol oxidase, EC 1.1.3.194-hydroxymandelate oxidase, EC 1.1.3.20 long-chain-alcohol oxidase, EC 1.1.3.21 glycerol-3-phosphate oxidase, EC 1.1.3.23 thiamin oxidase, EC 1.1.3.27 hydroxyphytanate oxidase, EC 1.1.3.28 nucleoside oxidase, EC 1.1.3.29 N-acylhexosamine oxidase, EC 1.1.3.30 polyvinyl-alcohol oxidase, EC 1.1.3.37 D-arabinono-1,4-lactone oxidase, EC 1.1.3.38 vanillyl-alcohol oxidase, EC 1.1.3.39 nucleoside oxidase (H₂O₂-forming), EC 1.1.3.40 D-mannitol oxidase, and EC 1.1.3.41 alditol oxidase.

EC number 1.2.3: EC number 1.2.3 includes oxidoreductases that act on the aldehyde or oxo group of donors with oxygen as an acceptor such as: EC 1.2.3.1 aldehyde oxidase, EC 1.2.3.3 pyruvate oxidase, EC 1.2.3.4 oxalate oxidase, EC 1.2.3.5 glyoxylate oxidase, EC 1.2.3.6 pyruvate oxidase (CoA-acetylating), EC 1.2.3.7 indole-3-acetaldehyde oxidase, EC 1.2.3.8 pyridoxal oxidase, EC 1.2.3.9 aryl-aldehyde oxidase, EC 1.2.3.11 retinal oxidase, EC 1.2.3.12 vanillate demethylase, EC 1.2.3.134-hydroxyphenylpyruvate oxidase, and EC 1.2.3.14 abscisic aldehyde oxidase.

EC number 1.3.3: EC number 1.3.3 includes oxidoreductases that act on the CH—CH group of donors with oxygen as an acceptor such as: EC 1.3.3.3 coproporphyrinogen oxidase, EC 1.3.3.4 protoporphyrinogen oxidase, EC 1.3.3.5 bilirubin oxidase, EC 1.3.3.6 acyl-CoA oxidase, EC 1.3.3.7 dihydrouracil oxidase, EC 1.3.3.8 tetrahydroberberine oxidase, EC 1.3.3.9 secologanin synthase, EC 1.3.3.10 tryptophan α,β-oxidase, EC 1.3.3.11 pyrroloquinoline-quinone synthase, and EC 1.3.3.12 L-galactonolactone oxidase.

EC number 1.4.3: EC number 1.4.3 includes oxidoreductases that act on the CH—NH₂ group of donors with oxygen as an acceptor such as: EC 1.4.3.1 D-aspartate oxidase, EC 1.4.3.2 L-amino-acid oxidase, EC 1.4.3.3 D-amino-acid oxidase, EC 1.4.3.4 amine oxidase, EC 1.4.3.5 pyridoxal 5′-phosphate synthase, EC 1.4.3.7 D-glutamate oxidase, EC 1.4.3.8 ethanolamine oxidase, EC 1.4.3.10 putrescine oxidase, EC 1.4.3.11 L-glutamate oxidase, EC 1.4.3.12 cyclohexylamine oxidase, EC 1.4.3.13 protein-lysine 6-oxidase, EC 1.4.3.14 L-lysine oxidase, EC 1.4.3.15 D-glutamate(D-aspartate) oxidase, EC 1.4.3.16 L-aspartate oxidase, EC 1.4.3.19 glycine oxidase, EC 1.4.3.20 L-lysine 6-oxidase, EC 1.4.3.21 primary-amine oxidase, EC 1.4.3.22 diamine oxidase, and EC 1.4.3.23 7-chloro-L-tryptophan oxidase.

EC number 1.5.3: EC number 1.5.3 includes oxidoreductases that act on the CH—NH group of donors with oxygen as an acceptor such as: EC 1.5.3.1 sarcosine oxidase, EC 1.5.3.2 N-methyl-L-amino-acid oxidase, EC 1.5.3.4 N6-methyl-lysine oxidase, EC 1.5.3.5 (S)-6-hydroxynicotine oxidase, EC 1.5.3.6 (R)-6-hydroxynicotine oxidase, EC 1.5.3.7 L-pipecolate oxidase, EC 1.5.3.10 dimethylglycine oxidase, EC 1.5.3.12 dihydrobenzophenanthridine oxidase, EC 1.5.3.13 N1-acetylpolyamine oxidase, EC 1.5.3.14 polyamine oxidase (propane-1,3-diamine-forming), EC 1.5.3.15 N8-acetylspermidine oxidase (propane-1,3-diamine-forming), EC 1.5.3.16 spermine oxidase, EC 1.5.3.17 non-specific polyamine oxidase, and EC 1.5.3.18 L-saccharopine oxidase.

EC number 1.6.3: EC number 1.6.3 includes oxidoreductases that act on NADH or NADPH with oxygen as an acceptor such as EC 1.6.3.1 NAD(P)H oxidase.

EC number 1.7.3: EC number 1.7.3 includes oxidoreductases that act on other nitrogenous compounds as donors with oxygen as an acceptor such as: EC 1.7.3.1 nitroalkane oxidase, EC 1.7.3.2 acetylindoxyl oxidase, EC 1.7.3.3 factor-independent urate hydroxylase, EC 1.7.3.4 hydroxylamine oxidase, and EC 1.7.3.5 3-aci-nitropropanoate oxidase.

EC number 1.8.3: EC number 1.8.3 includes oxidoreductases that act on a sulfur group of donors with oxygen as an acceptor such as: EC 1.8.3.1 sulfite oxidase, EC 1.8.3.2 thiol oxidase, EC 1.8.3.3 glutathione oxidase, EC 1.8.3.4 methanethiol oxidase, EC 1.8.3.5 prenylcysteine oxidase, and EC 1.8.3.6 farnesylcysteine lyase.

EC number 1.9.3: EC number 1.9.3 includes oxidoreductases that act on a heme group of donors with oxygen as an acceptor such as EC 1.9.3.1 cytochrome-c oxidase.

EC number 1.10.3: EC number 1.10.3 includes oxidoreductases that act on diphenols and related substances as donors with oxygen as an acceptor such as: EC 1.10.3.1 catechol oxidase, EC 1.10.3.2 laccase, EC 1.10.3.3 L-ascorbate oxidase, EC 1.10.3.4 o-aminophenol oxidase, EC 1.10.3.53-hydroxyanthranilate oxidase, EC 1.10.3.6 rifamycin-B oxidase, EC 1.10.3.9 photosystem II, EC 1.10.3.10 ubiquinol oxidase (H⁺-transporting), EC 1.10.3.11 ubiquinol oxidase, and EC 1.10.3.12 menaquinol oxidase (H⁺-transporting).

EC number 1.16.3: EC number 1.16.3 includes oxidoreductases that oxidize metal ions with oxygen as an acceptor such as EC 1.16.3.1 ferroxidase.

EC number 1.17.3: EC number 1.17.3 includes oxidoreductases that act on CH or CH₂ groups with oxygen as an acceptor such as: EC 1.17.3.1 pteridine oxidase, EC 1.17.3.2 xanthine oxidase, and EC 1.17.3.36-hydroxynicotinate dehydrogenase.

EC number 1.21.3: EC number 1.21.3 includes oxidoreductases that act on X—H and Y—H to form an X—Y bond with oxygen as an acceptor such as: EC 1.21.3.1 isopenicillin-N synthase, EC 1.21.3.2 columbamine oxidase, EC 1.21.3.3 reticuline oxidase, EC 1.21.3.4 sulochrin oxidase [(+)-bisdechlorogeodin-forming], EC 1.21.3.5 sulochrin oxidase [(−)-bisdechlorogeodin-forming, and EC 1.21.3.6 aureusidin synthase.

EC number 3.2.1.23: EC number 3.2.1.23 includes enzymes that are hydrolases, including glycosylases and glycosidases, i.e. enzymes hydrolysing O- and S-glycosyl compounds such as: EC 3.2.1.23 β-galactosidase.

Carbohydrate oxidase: Carbohydrate oxidases include enzymes classified under EC 1.1.3. Carbohydrate oxidase refers to oxidases that use at least oxygen and a carbohydrate as reactants. Non-limiting examples of carbohydrate oxidases include oxidases such as hexose oxidases which are capable of oxidizing several saccharides including glucose, galactose, maltose, cellobiose, and lactose. Additional examples of carbohydrate oxidases include monosaccharide oxidases, oligosaccharide oxidases and polysaccharide oxidases.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “means” as used herein shall be given its broadest possible interpretation in accordance with 35 U.S.C. §112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### Advantages

Advantages in using biosensing systems for measuring analytes include fast measurement, generally on the order of minutes. This is a big advantage over traditional methods like GC or HPLC in which a lot of time is spent in collection of the sample and extraction of analytes from the sample.

Small size is another advantage of using biosensing systems. Biosensing systems and biosensing elements of the present disclosure have a compact design for field use and are therefore capable of measurements in confined places such as needles and catheters in vivo and in conditions where weight is critical like spacecraft or airplanes.

Another advantage of using biosensing systems is that they can be used to measure multiple analytes in a small sample in a continuous real-time measurement in a reversible manner with extremely low signal loss in an optical fiber as compared to electronic sensors such as amperometric assays. Furthermore, biosensing systems are capable of measuring at greater depths such as taking measurements in groundwater monitoring.

An advantage is the ability of biosensing systems to measure complex samples with no prior preparation of samples, no addition of the reagents in the samples. Biosensing systems can provide direct measurements in blood, food, and waste water, for example. This is important as removal of the sample from its environment (as in case of analyses by GC or HPLC) can change its chemistry and can thereby lead to inaccurate results. Also, this eliminates and simplifies sample separation steps and reduces the cost of the process. Measurements using biosensing systems can be made with minimum perturbations of the sample.

Biosensing systems have high specificity and sensitivity for measuring analytes of interest. Although traditional methods such as GC or HPLC may be very sensitive, they rely upon separating compounds before they are able to detect and identify the compounds. Other methods such as solid-phase enzyme immunoassay (ELISA) may be both sensitive and specific, but may not be as cost effective as a biosensing system, portable for field use or able to perform continuous, in-situ measurements.

Another advantage for using biosensing systems of the present disclosure is the low cost of mass production compared to most of the traditional methods like GC or HPLC. Biosensing systems of the present disclosure are easy to use compared to traditional measurement techniques such as gas chromatography, ion-chromatography and high-pressure liquid chromatography. Biosensing systems using the proper biocomponents can also measure the toxicity of chemicals whereas analytical methods such as GC and HPLC can only measure concentration.

### Biocomponents

Biocomponents react with, bind to or otherwise interact with an analyte. Reactive biocomponents produce or react with atoms, molecules or compounds that interact with the transducer.

Enzymes are proteins that can serve as biocomponents that catalyze reactions with their substrates. Substrates may be analytes. The products or reactants of the enzymatic reactions are usually measured by the biosensing system. In one embodiment, the products of the substrates that react with the analyte may themselves be acted upon and thereby produce additional products which may be measured by the biosensing system. Therefore, a biosensing system may measure primary, secondary or even higher orders of products caused by an initial reaction or binding of the analyte with the biocomponent.

Generally, enzymes for use in biosensing systems may be disposed within whole cells or extracted from cells and purified. Whole cells and microorganisms are also biocomponents and are generally less expensive than purified enzymes and may provide an environment for longer enzyme stability. The cells and organisms used as biocomponents may or may not be living (able to replicate). Whether or not the cells are living, diffusion mechanisms and membrane-bound pumps may still be active that allow for the exchange of analytes and other compounds with the environment of the cell. It is often advantageous to use a dead cell or microorganism as a biocomponent at least because the proteolytic enzymes and pathways operating in a living cell generally cease to function and the enzymes, for example, that are responsible for binding or reacting with the analytes therefore last longer than they would in a living cell. Another advantage of using dead cells or microorganisms is that if the biosensing system is used in-situ, such as in-line testing of milk being produced at a factory, there can be no contamination of the sample with cells or microorganisms that may infect or adulterate the sample.

Purified enzymes may be used as a biocomponent in biosensing systems. Often, the extraction, isolation and purification of a particular enzyme can be expensive. Additionally, enzymes often lose their activity when separated from their intracellular environment that provides structural proteins, co-factors, consistent pH levels, buffers and other factors that contribute to the molecular integrity of the enzyme. Some enzymes are more robust than others. For example, enzymes isolated from extremophilic organisms such as hyperthermophiles, halophiles, and acidophiles often are more resistant to being exposed to environments substantially different from those found inside of a cell or microorganism. Extracellular enzymes are also usually more robust than enzymes that are membrane bound or solely exist within the cytosol.

Using only purified enzymes, in comparison to whole cells, can lead to increased sensor response by performing the desired reaction. A whole cell contains many enzymes, some of which may react with the analyte in a manner that does not yield the desired sensor signal. For example, a whole cell might also contain glucose dehydrogenase, which catalyzes a different, undesired reaction in which glucose is consumed but no change in oxygen concentration results. Immobilizing only the enzyme that catalyzes the desired reaction eliminates competing, undesired reactions and increases the sensor response to the analyte.

Using purified enzymes can lead to increased sensor response by increasing the concentration of immobilized enzyme. The concentration of an enzyme present in the detection layer on a sensor tip influences the response range of that sensor. Purified enzymes can be added at a higher concentration than enzyme contained in whole cells because the other components of the cell (normally the desired enzyme comprises <10% of the cell mass) occupy space in the matrix that could be occupied by the desired enzyme.

Using purified enzymes can lead to increased sensor selectivity. Sensor selectivity is the ability to report the concentration of a certain analyte in the presence of other chemicals. By immobilizing only the desired detection enzyme on the end of a sensor, the desired reaction that occurs is the one catalyzed by the detection enzyme on the target analyte. In contrast, a whole cell contains many different enzymes, some of which may catalyze reactions that create the same chemical changes on the sensor tip as does the desired enzyme with the target analyte. For example, a cell might contain both glucose oxidase and L-amino acid oxidase. If the solution that is monitored contains not only glucose but also one or more L-amino acids, a sensor based on whole cells would respond to both chemicals in a manner that would be unknown to the user.

Using purified enzymes can enhance control of sensor manufacturing. Repeatable sensor production requires the application of an identical quantity of enzyme on each sensor tip. In contrast, when cells containing the detection enzyme are cultivated, the amount of this enzyme in each cell may vary. The exact amount of a purified enzyme can therefore be known and the correct amount applied to each sensor tip in a highly reproducible manner, in comparison to applying whole cells.

Although isolated and/or purified enzymes have many functional advantages, the ability of an isolated and/or purified enzyme to catalyze its corresponding chemical reaction under certain conditions is unpredictable. The ability of a purified enzyme to catalyze a reaction is dependent on a variety of factors, such as pH, temperature, thermostability characteristics, genetic sequence, source or origin, and the conditions under which the enzyme was purified and/or isolated. For example, in some cases, two identical oxidase enzymes (e.g., same genetic sequence, source or origin, etc.), will not necessarily function to catalyze the same oxidation reaction, even if the factors under which the reaction takes place are identical. Therefore, experimentation is typically required to determine if a specific enzyme will function to catalyze a reaction in any given assay. Also, if the desired enzyme is not available commercially, it must be produced, for example, in cell cultures and subsequently purified; this purification process may be complex and some enzyme activity may be lost. Additionally, enzymes may be less stable in their purified form than in their native physiological environment, such as a whole cell.

An enzyme's resistance to becoming inactivated due to environmental factors, or even by the nature of the reaction that they catalyze, may be increased through mutagenic techniques. Such techniques are well known in the art and include various incarnations of changing the coding nucleotide sequence for the protein through various techniques. The proteins produced by expressing the mutagenic nucleotide sequences may then be tested for resistance to environmental factors and/or increased reactivity with substrates. Such an increase in reactivity may be due to advantageous binding specificity and/or increased kinetics of the binding and/or reaction catalyzed by the enzyme.

Methods of choosing cells and microorganisms that increase the response of the biosensing system may also be used to create biosensing systems that possess increased sensitivity, have quicker response times and last longer. Such techniques include directed evolution and using micro-assays to determine an increase in the production amount and/or rate of production of the molecules and/or atoms that react with the transducer layer.

### Transducers

A transducer is a device or compound which converts an input signal into an output signal of a different form. A transducer may convert a chemical input signal into an optical output signal, for example. A transducer may also be a device or compound that receives energy from one system and supplies energy of either the same or of a different kind to another system, in such a manner that the desired characteristics of the energy input appear at the output. In a reaction-based biosensing system, a transducer is a substance or device that interacts with the atoms, compounds, or molecules produced or used by the biocomponent. The interaction of the transducer with the atoms, compounds, or molecules produced or used by the biocomponent causes a signal to be generated by the transducer. The transducer may also generate a signal as an inherent property of the transducer. The signal may be an electrical current, a photon, a luminescence, or a switch in a physical configuration. In one embodiment, the signal produced by the transducer is quenched by a reactant or product of the biocomponent. A transducer is a device that produces a measurable signal, or change in signal, upon a change in its chemical or physical environment. Transducers suited for biosensing systems that use enzymes as the biocomponent are those that interact with the reactants and/or products of the biocomponent and send a signal that is processed into a measurement reading. The nature of the interaction of the biological element with the analyte has a major impact on the choice of transduction technology. The intended use of the biosensing system imposes constraints on the choice of suitable transduction technique.

Amperometric transducers work by maintaining a constant potential on the working electrode with respect to a reference electrode, and the current generated by the oxidation or reduction of an electroactive species at the surface of the working electrode is measured. This transduction method has the advantage of having a linear response with a relatively simple and flexible design. Also, the reference electrode need not be drift-free to have a stable response. Since the signal generated is highly dependent on the mass transfer of the electroactive species to the electrode surface there can be a loss in sensitivity due to fouling by species that adsorb to the electrode surface. As a result of fouling, use of amperometric transducers is restricted where continuous monitoring is required. Enzymes, particularly oxidoreductases, are well suited to amperometric transduction as their catalytic activity is concerned with electron transfer.

Electroactive species that can be monitored at the electrode surface include substrates of a biological reaction (e.g., O₂, NADH), final products (e.g., hydrogen peroxide for oxidase reactions, benzoquinone for phenol oxidation) and also electrochemical mediators that can directly transfer electrons from the enzyme to the working electrode surface (e.g., hexacyanoferrate, ferrocene, methylene blue).

Potentiometric transducers work by having a potential difference between an active and a reference electrode that is measured under the zero current flow condition. The three most commonly used potentiometric devices are ion-selective electrodes (ISEs), gas-sensing electrodes and field-effect transistors (FETs). All these devices obey a logarithmic relationship between the potential difference and the activity of the ion of interest. This makes the sensors have a wide dynamic range. One disadvantage of this transducer is the requirement of an extremely stable reference electrode. Ion selective electrodes are commonly used in areas such as water monitoring. FETs are commercially attractive as they can be used to make miniaturized sensors, but manufacturing cost of FETs are high. Examples of potentiometric sensors are for acetaldehyde and cephalosporins, where the sensing electrode measures pH. Other examples are sensors used to measure creatinine, glutamine and nitrate with the sensing electrode detecting NH₃ gas.

Conductimetric transducers are often used to measure the salinity of marine environments. Conductance is measured by the application of an alternating current between two noble metal electrodes immersed in the solution. Due to specific enzyme reactions, they convert neutral substrates into charged products, causing a change in the conductance of the medium. This method can be used to make more selective and informative sensors by using multi-frequency techniques.

Optical transducers use optical phenomena to report the interaction of the biocomponent and the analyte. The main types of photometric behavior which have been exploited are ultraviolet and visible absorption, luminescence such as fluorescence and phosphorescence emission, bioluminescence, chemiluminescence, internal reflection spectroscopy using evanescent wave technology and laser light scattering methods.

One embodiment of an optical transducer uses luminescent reagents. In optical transducers that use luminescent reagents, a luminescent substance is excited by incident light, and as a result it emits light of longer wavelength. The intensity and/or lifetime decay of emitted light changes when an atom, molecule or compound binds or otherwise interacts with the luminescent substance. The atom, molecule or compound may be a reactant or product of the biocomponent. Thus, if a reactant or product of the biocomponent reacts with the luminescent transducer and affects the intensity and/or lifetime decay of the light emitted by the transducer layer, the change in the measurement of the intensity and/or lifetime decay can be measured as a response to a particular analyte. There are several luminescent reagents that may be useful as optical transducers. Examples include Tris(4,7-diphenyl-1,10-phenanthroline)Ru(H) chloride, also known as RuDPP, for oxygen sensors, trisodium 8-hydroxy-1,3,6-trisulphonate fluorescein, fluoresceinamine and other compounds containing fluorescein for pH sensors, fluoro(8-anilino-1-naphthalene sulphonate) for Na+ ion sensor and acridinium- and quinidinium-based reagents for halides.

Chemiluminescent and bioluminescent sensors work on principles similar to fluorescent sensors Chemiluminescence occurs by the oxidation of certain substances, usually with oxygen or hydrogen peroxide, to produce visible light. Bioluminescence is, for example, the mechanism by which light is produced by certain enzymes, such as luciferase.

Calorimetric transducers use the heat generated from biological reactions and correlate it with the reaction conditions. In order to measure such small amounts of heat liberated during the reaction, a very sensitive device is required. In the calorimetric technique a very sensitive, electrical resistance thermometer is used to detect temperature changes down to 0.001° C. This method is advantageous, as it is independent of the chemical properties of the sample. Calorimetric transduction has been used in a wide range of areas, including clinical chemistry, determination of enzyme activity, monitoring gel filtration, chromatography, process control and fermentation.

An acoustic transducer uses materials such as piezoelectrics as a sensor transducer due to their ability to generate and transmit acoustic waves in a frequency-dependent manner. The optimal resonant frequency for acoustic-wave transmission is highly dependent on the physical dimensions and properties of the piezoelectric crystal. Any change in the mass of the material at the surface of the crystal will cause quantifiable changes in the resonant frequency of the crystal. There are two types of mass-balance acoustic transducers: bulk wave and surface acoustic wave. Acoustic transduction is a relatively cheap technique but it has the disadvantage of having low sensitivity with non-specific binding. This technique is commonly used to measure the concentration of volatile gases and vapors. A piezoelectric immunobiosensor for measuring an analyte of interest in drinking water may use a piezoelectric crystal coated with polyclonal antibodies that bind to that analyte. When the analyte molecules come into contact with the antibodies, they bond with the antibodies causing a change in the crystal mass, which in turn leads to a shift in the oscillation frequency and produces a measurable signal that can be measured and correlated to the concentration of the analyte of interest within the sample.

### Optical and Signal Processing Systems

In an embodiment, biosensing systems of the present disclosure have a biocomponent, a transducer, a photon-detection device, and a signal-processing system. A signal processing system processes the signal from a photon-detection device into information that can be displayed to an end user. An example of a signal processing system is a microprocessor that accepts an input signal from a photon-detection device that is coupled to a biosensing element. The signal processing system then uses a software program that encodes an algorithm. The algorithm used by the software transforms the data provided by the input signal and provides an output signal that correlates to a numerical display of the concentration of an analyte that the biosensing system detected.

In an embodiment of the present disclosure, a biosensing system comprises a biocomponent attached to a fiber optic pH optode, lens focusing system, photomultiplier (PMT), analog/digital (A/D) converter and a microprocessor. The biosensing system may contain a biosensing element that is coupled to a polymethylmethacrylate (PMMA) optical fiber optic. The length of this connecting optical fiber may vary from 1 mm to well over 1 km. In an embodiment, the other end of this cable is attached to a metal casing containing a 5 W halogen lamp or other light source and a lens focusing system. The light source should be able to operate at high temperatures, having a very short warm-up time in order to reach a constant power output. In one embodiment, light from the halogen lamp is first passed through a bandpass filter such as a 480-nm bandpass filter, for example. The light is then collected, paralleled and focused to the tip of fiber optic cable using a lens focusing system. An embodiment of the lens focusing system comprises spheric, aspheric, and convex lenses, and a dichroic mirror. Light from the lamp that radiates in opposite directions to the lens system may be refocused by the spheric lens and paralleled by the aspheric lens.

When light, for example light at 480 nm, is incident on a sensing tip coated with PVA/fluoresceinamine dye, fluorescence occurs. In an embodiment, this light is then passed back through a 520 nm bandpass filter or other bandpass filter having a frequency of light that is either blue or red shifted in comparison to the incident light wavelength, paralleled by focusing lens and then directed by the dichroic mirror onto the window of a single channel photo-detection device. The change in intensity and/or lifetime decay properties of the light can be measured. The photon detection device processes this light and the output potentiometric signal is sent to a computer interface using a connector block where it was converted into a digital signal by a data acquisition card. The final output is observed on a computer using software such as LabView software or other algorithmic software that interprets the signals from the sensing tip and processes them into correlating concentration measurements of the atom, compound, molecule or analyte of interest.

In one embodiment, the transducer of the biosensing element uses an evanescent wave to detect the luminescence of a reagent of the transducer. The evanescent wave could result from a carrier wave propagating within a planar waveguide or fiber optical cable. The carrier wave could be coupled to a photon-detection device that measures the interference of the evanescent wave with the carrier wave. This interference would correlate to the activity of the transducer and therefore the activity of the biocomponent and thus the concentration of an analyte of interest could be calculated from measuring the interference of the carrier wave within the planar waveguide.

### Biosensing Elements

This disclosure embodies an optical enzymatic biosensing system for lactose and hydrogen peroxide. Several biosensing system designs are disclosed herein including biosensing elements on the tip of a fiber optical cable, and biosensing elements displaced upon a surface, for example. The biosensing system may be based on an optical pH or optical oxygen sensor. Carbohydrate oxidase may be used alone as the biocomponent or in conjunction with catalase. The biosensing elements may be separate from one another or combined into the same tip or biosensing element.

Some enzymes that react with lactose, such as carbohydrate oxidase, produce hydrogen peroxide as a by-product. In one embodiment, hydrogen peroxide can then be detected in the biosensing element and used as an indicator of the concentration of lactose in the aqueous solution. Some biosensing systems are made using food-grade enzymes and materials. These biosensing systems are advantageously used for measuring analytes in milk or other food products.

The disclosure presented herein is a set of biosensing system designs based on optical transduction. Optical enzymatic biosensing system designs using an optical signal transaction are more robust and less susceptible to chemical interference than electrochemical (e.g., amperometric) methods. In one embodiment, optical pH and optical oxygen sensors (optodes) employ fluorophores that are sensitive to either protons (H⁺ ions) or molecular oxygen. Optical enzymatic biosensing elements are formed by combining a transducer and/or optode with a biocomponent that catalyzes a reaction with the analyte and results in altered pH or oxygen levels.

### Hydrogen Peroxide as an Analyte

Hydrogen peroxide may be involved with, used, or produced in various processes in the dairy industry. Hydrogen peroxide is often used in food production to sterilize lines, including those carrying various foods and food ingredients. For example, lines that carry milk, processing vessels, and milk jugs are sterilized prior to filling to kill bacteria and prevent contamination of the fresh milk. Although hydrogen peroxide is not supposed to reach the consumer, sometimes the milk can arrive contaminated. Hydrogen peroxide has been shown to cause damage to the heart, lungs, arteries and veins upon ingestion. While the concentrations in milk are not likely to be fatal, the possibility of side effects still exists, and milk should be checked to ensure that it is safe to consume. This is particularly important since milk is a common drink for babies and small children.

### Measuring Oxygen Generated by Catalase

In one embodiment, catalase is used as a biocomponent coupled to an oxygen optode that measures a change in the concentration of oxygen in the solution. Catalase catalyzes the decomposition of hydrogen peroxide into water and oxygen. Thus, when hydrogen peroxide is in a solution and interacts with the biosensing element, oxygen is produced. The oxygen produced interacts with the transducer by quenching some of the luminescence of the transducer. Thus, the transducer produces a signal that is correlated to the concentration of oxygen in the sample which is related to the concentration of hydrogen peroxide.

### Lactose as an Analyte

Several enzymes that react directly with lactose produce or consume an atom, molecule or compound that can be measured directly by the biosensing system are discussed herein. Additionally, several enzymes that react with at least one of the products of the initial reaction with lactose and create at least one product or use a reactant that interacts with the transducer layer of the biosensing element are discussed herein. Enzymes from several different enzyme commission number codes may be used as biocomponents in the biosensing systems and biosensing elements of the disclosures presented herein. Enzymes for use in the biosensing systems and biosensing elements disclosed herein may be selected from the group consisting of EC numbers, 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, and 3.2.1.23. Examples of embodiments of biosensing systems and biosensing elements for measuring lactose as an analyte include the following:

Measuring Use of Oxygen from Oxidation of Glucose

Lactose can be a substrate for beta-galactosidase. Beta-galactosidase is an enzyme that hydrolyzes lactose into galactose and glucose. The glucose thereby generated may then be oxidized with glucose oxidase. When glucose oxidase reacts with glucose, hydrogen peroxide and a glucono-lactone are generated. Using this scheme, oxygen is used and hydrogen peroxide is generated when lactose is present in a solution. The concentration of oxygen can be measured by an oxygen optode to detect oxygen consumption. Therefore, the concentration of lactose in a solution correlates to consumption of oxygen and the production of hydrogen peroxide.

In another embodiment, catalase may be added to the biosensing element. A benefit of this system is that the hydrogen peroxide generated by the action of the oxidase actually inhibits the catalysis of the oxidase through non-specific inhibition caused by the breakdown of hydrogen peroxide into hydroxyl radicals that react with amino acid moieties on the oxidase. In this embodiment, the catalase quickly degrades the hydrogen peroxide that is generated through the activity of the oxidase.

### Measuring pH Changes Due to Glucono-Lactone Degradation

In an embodiment of the above reactions of lactose, a biosensing system may use a pH optode to measure the pH change caused by the production of the hydrogen ions produced by the spontaneous hydrolysis of the D-glucono-1,5-lactone generated by the action of glucose oxidase on the glucose created by the reaction of lactose with beta-galactosidase.

In another embodiment, catalase may be added to the biosensing element. A benefit of this system is that the hydrogen peroxide generated by the action of the oxidase actually inhibits the catalysis of the oxidase through non-specific inhibition caused by the breakdown of hydrogen peroxide into hydroxyl radicals that react with amino acid moieties on the oxidase. In this embodiment, the catalase quickly degrades the hydrogen peroxide that is generated through the activity of the oxidase.

### Measuring Use of Oxygen by the Oxidation of Galactose

In another embodiment, cleavage of lactose with beta-galactosidase is followed by oxidation of the produced galactose with galactose oxidase in a reaction that uses oxygen and generates hydrogen peroxide. The concentration of oxygen can be measured by an oxygen optode to detect oxygen consumption. Therefore, the concentration of lactose in a solution correlates to the consumption of oxygen and the production of hydrogen peroxide.

In another embodiment, catalase may be added to the biosensing element. A benefit of this system is that the hydrogen peroxide generated by the action of the oxidase actually inhibits the catalysis of the oxidase through non-specific inhibition caused by the breakdown of hydrogen peroxide into hydroxyl radicals that react with amino acid moieties on the oxidase. In this embodiment, the catalase quickly degrades the hydrogen peroxide that is generated through the activity of the oxidase. Generally, if the only source of hydrogen peroxide is the oxidase (detection enzyme) reaction, then the catalase or peroxidase can be co-immobilized with the oxidase to remove hydrogen peroxide rapidly after the hydrogen peroxide is formed. If the major source of hydrogen peroxide is the solution in which the measurement/monitoring is to be performed, the catalase or peroxidase can be included in a separate layer above the oxidase layer. In some cases, catalase and peroxidase can be both co-immobilized with an oxidase as well as placed in a layer above that.

### Measuring pH Changes Due to Galactono-Lactone Degradation

In an embodiment of the above reactions of lactose, a biosensing system may use a pH optode to measure the pH change caused by the production of the hydrogen ions produced by the spontaneous hydrolysis of the D-galactono-1,5-lactone produced by the action of galactose oxidase on the galactose created by the reaction of lactose with beta-galactosidase.

In another embodiment, catalase may be added to the biosensing element. A benefit of this system is that the hydrogen peroxide generated by the action of the oxidase actually inhibits the catalysis of the oxidase through non-specific inhibition caused by the breakdown of hydrogen peroxide into hydroxyl radicals that react with amino acid moieties on the oxidase. In this embodiment, the catalase quickly degrades the hydrogen peroxide that is generated through the activity of the oxidase.

### Measuring Oxygen Use by Carbohydrate Oxidase

In an embodiment, carbohydrate oxidase oxidizes lactose while using oxygen to create a lactone and hydrogen peroxide. Thus, the use of oxygen is measured and correlated to the concentration of lactose in the solution. In another embodiment, the detection of the generation of hydrogen peroxide is correlated to the concentration of lactose in the solution either alone or in coordination with the detection of the use of oxygen.

In another embodiment, catalase may be added to the biosensing element. In this embodiment, the catalase quickly degrades the hydrogen peroxide that is generated through the activity of the oxidase.

### Measuring pH Changes Due to δ-Lactone Degradation

In an embodiment of the above reaction of lactose with carbohydrate oxidase, a biosensing system may use a pH optode to measure the pH change caused by the production of the hydrogen ions produced by lactobionic acid created from the spontaneous hydrolysis of the enzymatic product δ-lactone.

In another embodiment, catalase may be added to the biosensing element. A benefit of this system is that the hydrogen peroxide generated by the action of the oxidase actually inhibits the catalysis of the oxidase through non-specific inhibition caused by the breakdown of hydrogen peroxide into hydroxyl radicals that react with amino acid moieties on the oxidase. In this embodiment, the catalase quickly degrades the hydrogen peroxide that is generated through the activity of the oxidase.

### Measuring Net Oxygen Consumption by Carbohydrate Oxidase and Catalase and Measuring pH Changes Due to the Degradation of the Lactone in the Same Biosensing Element

In an embodiment, carbohydrate oxidase reacts with lactose to use oxygen and generate hydrogen peroxide, and the hydrogen peroxide generated then reacts with catalase to form water and oxygen. A benefit of this system is that the hydrogen peroxide generated by the action of carbohydrate oxidase actually inhibits the catalysis of carbohydrate oxidase through non-specific inhibition caused by the breakdown of hydrogen peroxide into hydroxyl radicals that react with amino acid moieties on carbohydrate oxidase. Thus, using this embodiment, the protons generated through the spontaneous degradation of the lactone change the pH of the solution. The measurement of the pH of the solution is therefore correlated to the concentration of lactose in the sample. One advantage of using this co-system of both carbohydrate oxidase and catalase is that the oxygen substrate is generated through the degradation of the inhibitory hydrogen peroxide. Thus, oxygen is recycled in the system and hydrogen peroxide is broken down before it can degrade carbohydrate oxidase.

Measuring pH from Cellobiose Dehydrogenase Activity

In an embodiment, cellobiose dehydrogenase reacts with lactose and flavin adenine dinucleotide, reducing flavin adenine dinucleotide and oxidizing cellobiose into cellobiono-1,5-lactone and generating protons. The protons cause a change in the pH of the solution which is measured and correlated to the concentration of lactose in the solution.

### Using a Carbohydrate Biosensing System and Hydrogen Peroxide Biosensing System in Tandem

In yet another embodiment, a carbohydrate biosensing system and a hydrogen peroxide biosensing system may be used concurrently within the same sample but in different biosensing elements. In such an embodiment, hydrogen peroxide and carbohydrate concentrations are each measured by distinct biosensing elements or by a biosensing element that has both catalase and one or more of carbohydrate oxidase, beta-galactosidase, glucose oxidase, galactose oxidase, carbohydrate oxidase or cellobiose dehydrogenase.

As an example of an embodiment able to measure both pH and oxygen at the same time within the same biosensing system, catalase would react with hydrogen peroxide to produce oxygen which interacts with an oxygen-sensitive transducer layer on the biosensing element while cellobiose dehydrogenase reacts with lactose to produce a change in the pH of the solution which is measured by a pH-sensitive transducer layer on the same biosensing element. Thus one biosensing system simultaneously measures the concentrations of two different analytes that correlate to the concentrations of two different compounds of interest, here lactose and hydrogen peroxide.

### Biosensing System Detection Range of Hydrogen Peroxide Biosensing Element

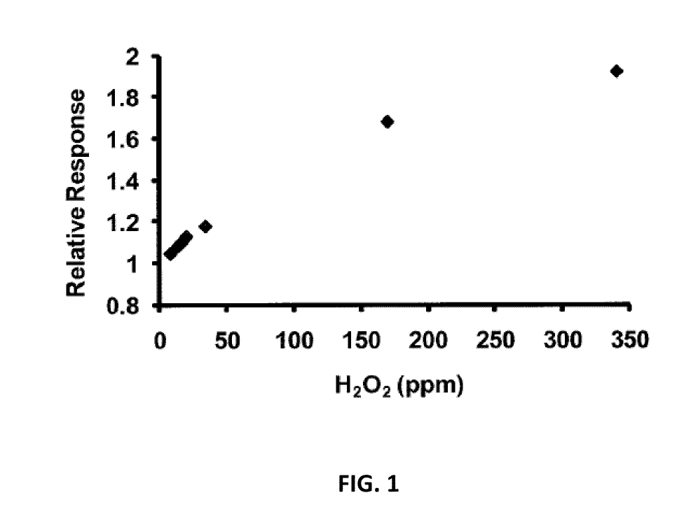

Biosensing systems were tested in the concentration range of 8-340 ppm H₂O₂ (340 ppm H₂O₂ is the same as 10 mM H₂O₂), see FIG. 1. This biosensing system gave a linear response from 8 to 170 ppm H₂O₂, with increasing but nonlinear response at higher concentrations of analyte.

In another example, the linear range of this biosensing system may be extended to about 60 mM by using a variety of different techniques to construct the biosensing element such as through various immobilization techniques and/or various cross-linking techniques.

### Biosensing System Detection Range of Lactose Sensor

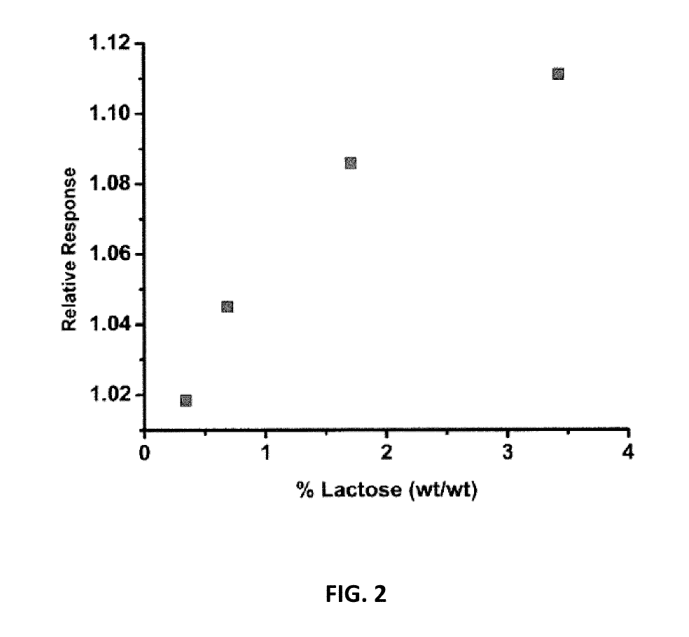

Biosensing systems were tested in the concentration range of 0.014-3.4% (wt/wt) lactose using biosensing elements and systems engineered for the lower and higher end of this concentration regime, respectively. Both lower concentration and higher concentration biosensing element types showed linear concentration dependence over specific concentration windows. The response of the higher concentration biosensing system is shown in FIG. 2. This particular biosensing system gave a linear response for lactose concentrations up to 1.7%, and had signal saturation for concentrations above this threshold.

In a prophetic example, the linear range of this biosensing system may be extended to at least 20% lactose by using a variety of different techniques to construct the biosensing system such as through various immobilization techniques and/or various cross-linking techniques.

### Biosensing System Detection at High Analyte Concentrations

An analysis of lactose concentrations that is in-line with the processing of milk would save money and time involved in sending the samples to a lab for analysis while also allowing for the adjustment of processing the milk at the factory where the processing could easily be shifted towards another product or changed according to the reading of the lactose concentration of the milk.

Some biosensing system applications may require the measurement of relatively high analyte concentrations, such as the measurement of lactose in milk (ca. 5% by weight, or 50 g/L) or ethanol content of beer (ca. 6% by weight, or 60 g/L). These concentrations are high enough to saturate the response of the biocomponent, meaning that all of the binding sites of an antibody or all of the enzymatic reaction sites are occupied. Under these saturating conditions, the biosensing system response is no longer dependent upon the analyte concentration and no measurement can be made.

One embodiment of the present disclosure is for biosensing systems that contain biosensing elements that use enzymes as biocomponents and can be used to provide a linear response in high analyte concentrations. Biosensing elements for the measurement of analytes at high concentrations can be used in many scenarios (such as the food and beverage examples listed above) and the concepts are broadly applicable for the measurement of other analytes in other solutions such as the measurement of halogenated hydrocarbons, for example.

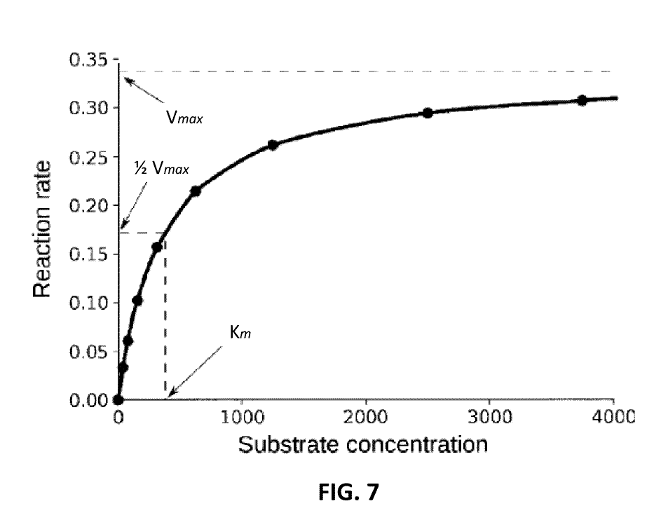

Biosensing elements using enzymes as biocomponents may be constructed as thin enzyme-containing films deposited or placed over the transducer/fluorescent chemical layer. The response of biosensing systems that use these biosensing elements (signal as a function of analyte concentration) is governed by the rate of the enzymatic reaction and the manner in which that rate depends on the analyte concentration. For most enzymes, this relationship is the saturation type shown in FIG. 7 and modeled by the Michaelis-Menten equation in which the rate depends nearly linearly on analyte concentration at low concentrations but becomes independent of concentration at high concentrations. The Michaelis-Menten equation describes the rate of enzymatic reactions by relating reaction rate ν to [S], the concentration of a substrate S. Vₘₐₓ is the maximum rate achieved by the system, at maximum (saturating) substrate concentrations. The Michaelis constant Kₘ is the substrate concentration at which the reaction rate is half of Vₘₐₓ. The equation, equation 1, is as follows:

$$v = \frac{{V}_{max} [ S ]}{{K}_{m} + [ S ]}$$

For biosensing element that has a thin-layer of enzyme biocomponent, this means that the biosensing element response becomes saturated and consequently it is not possible to distinguish one high concentration value from another.

To describe this high concentration range more accurately, it is convenient to use the Michaelis-Menten equation, which relates the enzymatic reaction rate R_{enz} to the concentration of the analyte (C_{A}) as represented in the following equation, equation 2; R_{enz}=kC_{E}C_{A}/K_{M}+C_{A} in which k and K_{M} are parameters of the enzymatic reaction rate (depending on the enzyme and the analyte) and C_{E} is the concentration of enzyme. The combined term kC_{E} is frequently presented as Vₘₐₓ, the maximum reaction rate (“velocity”). The Michaelis-Menten equation has been found to accurately describe many different enzyme-catalyzed reactions.

When analyte concentrations are low enough that C_{A} is much less than K_{M}, the Michaelis-Menten equation approximately reduces to a first-order (linear) dependence of the reaction rate on the analyte concentration, R_{enz}=(Vₘₐₓ/K_{M})C_{A} This linear response is the desired operating condition for a biosensing element. However, for biosensing elements that have a thin layer of enzyme biocomponent, this range extends only to values of C_{A} that are small relative to K_{M}; “small” can be interpreted as when C_{A} is 10% or less of K_{M}. At higher analyte concentrations, the relationship of the enzymatic reaction rate to the analyte concentration, and thus the relationship of the biosensing element response to the analyte concentration, becomes increasingly nonlinear. Once the analyte concentration becomes much larger than K_{M} such that C_{A}+K_{M}=C_{A}, the enzymatic reaction rate and the biosensing system response become essentially independent of C_{A}. Modifying the Michaelis-Menten equation for this case of C_{A}>>K_{M} yields R_{enz}=Vₘₐₓ.

The analysis above is based on the assumption that the analyte concentration in the vicinity of the enzyme molecules of the biocomponent layer (“local” concentration) is the same as in the solution in which the biosensing element is placed (“bulk solution” concentration). However, this situation can be manipulated such that the local concentration is lowered such that it falls within the linear measurement range. The local concentration can be related to the bulk solution concentration by either calculating the reaction-diffusion behavior of the system or through experimental calibration procedures.

A solution to extend the linear (useful) measurement range of biosensing elements that have an enzyme biocomponent beyond that available with thin-film designs is to add a mass transfer (diffusion) barrier. This diffusion barrier may take the form of a polymer coating, a membrane, or any other material through which the analyte passes more slowly than through the measurement medium. An effective diffusion barrier could also be created by increasing the thickness of the enzyme layer. Biosensing elements that have an increased thickness of the enzyme biocomponent layer are generally referred to as a thick-film biosensing element. Linear measurement ranges can be extended through the use of thick-film biosensing element designs. The rates of analyte mass transfer and reaction remain coupled in thick-film biosensing element designs. Thus, at some analyte concentration, the rate of mass transfer is high enough that the analyte concentration near the enzymes exceeds the linear reaction rate range and the biosensing system no longer has a direct, linear response to the analyte concentration.

In one embodiment, biosensing systems of the present disclosure use a design scheme for the construction of biosensing elements capable of measurements at high analyte concentrations. This is based on the combination of a high mass transfer resistance and a high biocomponent enzyme concentration, so that the analyte concentration near the transducer/fluorophore layer always remains in the linear reaction rate (and biosensing element response) range.

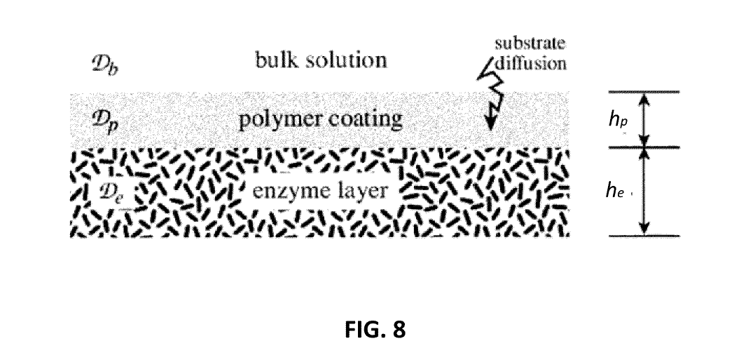

For any given concentration of any particular analyte, the appropriate ranges of the mass transfer coefficient of the analyte/substrate from the bulk solution to the enzyme biocomponent layer, and the reaction rate parameters of the enzyme layer, can be determined according to equation 3: ((Da+1−β)²/4β)>>1. Da is a dimensionless number used to relate chemical reaction timescales to other phenomena occurring in a system. Da represents a dimensionless reaction time. And where β=the substrate concentration in the bulk solution divided by K_{M} of the enzyme for the substrate; and where Da is (hₑVₘₐₓhₚ)/(DₚK_{M}) where hₑ is the thickness of the enzyme biocomponent layer which is embedded within a matrix; hₚ is the thickness of a porous polymeric or ceramic material which sits atop the enzyme biocomponent layer; where Dₚ is the diffusion coefficient of the polymer coating, see FIG. 8.

Therefore, by using equation 3, the calculations provide specific design parameters such as the thickness of the enzymatic biocomponent (detection) and mass transfer resistance layers such that a linear biosensing element and thus a linear biosensing system response is obtained for a given concentration, see FIG. 8.

In one embodiment of the present disclosure a method is used to provide the design parameters for constructing biosensing elements used in biosensing systems. The method uses a microprocessor that uses software encoding an algorithm that uses equation 3 to determine hₑ, the thickness of the enzyme biocomponent layer which is embedded within a matrix; the thickness of a porous polymeric or ceramic material hₚ, which sits atop the enzyme biocomponent layer; and a polymer coating that has the proper diffusion coefficient Dₚ, that can all be used to construct a biosensing element that has a linear response in a given range of analyte concentration in a solution.

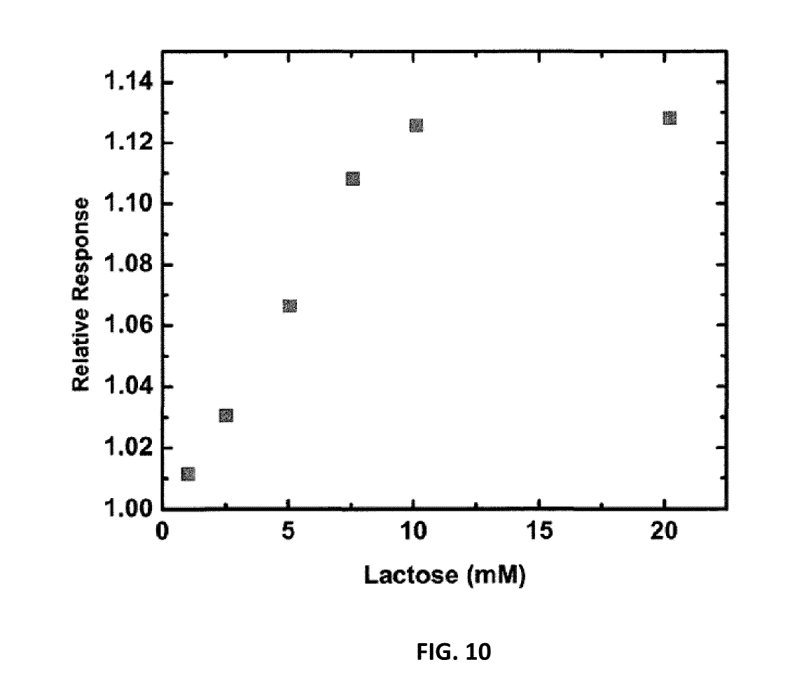

The effect of having differing membrane materials placed upon the top of an enzyme biocomponent thin film are exemplified in the following embodiments of the biosensing elements and biosensing systems of the present disclosure. In one embodiment, a lactose biosensing system includes only a thin film of enzyme biocomponent that is immobilized on the surface of the biosensing element. In another embodiment, the lactose biosensing system includes a porous membrane placed over the same thickness of enzyme biocomponent layer. In another embodiment, the lactose biosensing system can include the same thickness of enzyme biocomponent layer, as well as a membrane layer placed over its biosensing element that is less porous than the porous membrane of the biosensing element represented in FIG. 10.

In some embodiments, biosensing elements of the present disclosure can have a membrane material consisting of track-etched polycarbonate with a pore size of 0.015 μm. Additional mass transfer resistance can be provided for biosensing elements, for example, by casting a polyurethane coating on top of the porous membrane material.

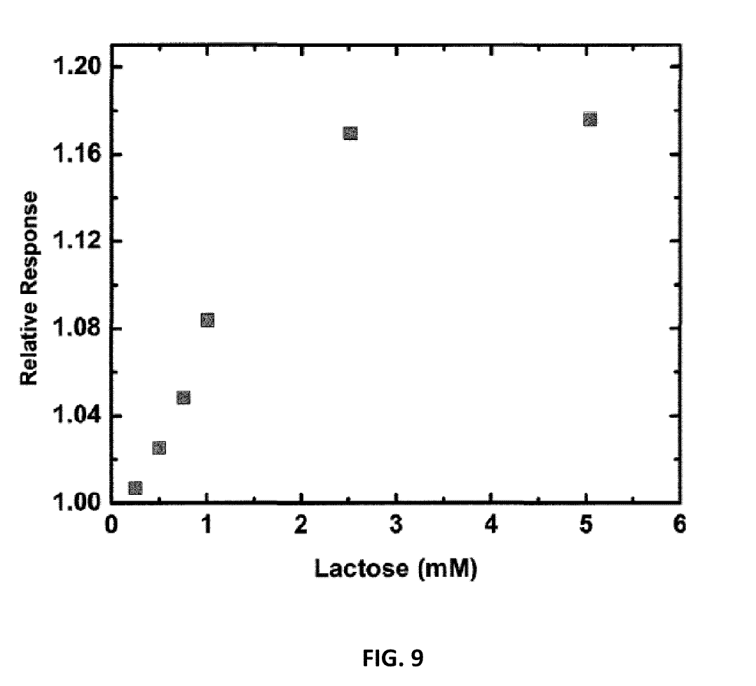

The response of a lactose biosensing system to a series of lactose standards is show in FIG. 9. The lactose biosensing element's response begins to saturate at concentrations above 1.01 mM lactose, and in some cases, up to and above 2.0 mM. Signal saturation is due to the presence of substrate/analyte at concentrations that exceed the K_{M} of the enzyme.

In some embodiments, a biosensing element includes a diffusion layer or diffusion barrier on top of the enzyme biocomponent layer. The diffusion layer can provide a region through which the analyte molecules must diffuse before reaching the detection enzyme layer. This layer can be made of any material through which the analyte molecules can diffuse, but do so at a slower rate than the material in which the detection (and/or enzymes such as catalase/peroxidase) enzymes are immobilized. This diffusion layer or barrier extended the linear detection range of the biosensing system into higher concentration ranges, as shown, for example, in FIG. 10. In some cases, a porous polycarbonate membrane can be immobilized on top of the enzyme biocomponent layer to act as barrier to analyte mass transfer, which can result in a lower analyte concentration in the enzyme biocomponent layer compared to that in bulk solution. In some cases, the diffusion layer serves to significantly restrict the diffusion of a undesirable chemical or contaminant that is found in the same solution as the analyte. For example, the diffusion layer can be selectively permeable, with higher permeability to the analyte and lower permeability to interfering chemicals. In some cases, diffusion layers are fabricated with polyurethane materials, such as HYDROTHANE and/or derivatives thereof. In other cases, diffusion layers are fabricated with fluoropolymer-copolymer materials, such as the sulfonated tetrafluoroethylene based fluoropolymer-copolymer, Nafion.

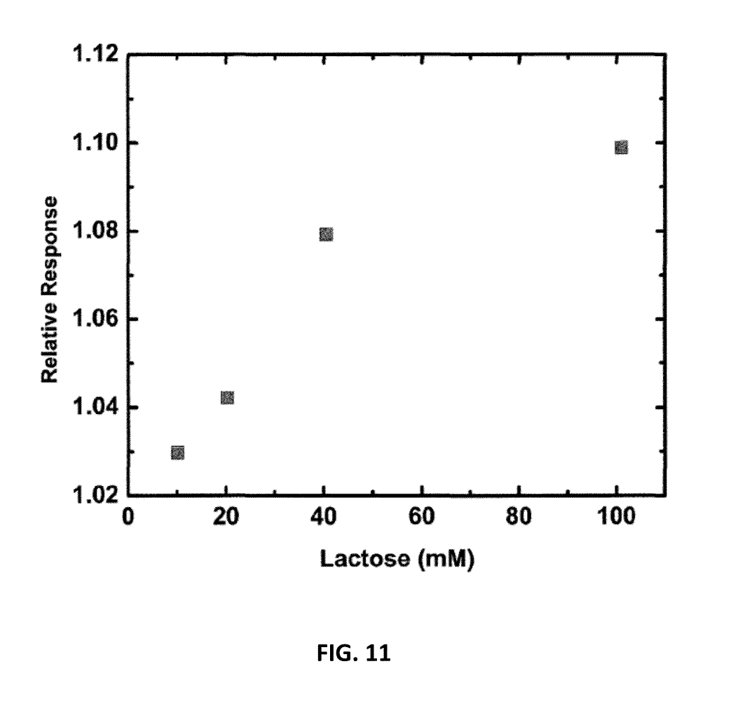

Various embodiments of the biosensing elements of the present disclosure can also include a less porous polycarbonate membrane, which can cause a decrease in the porosity of the diffusion layer and result in the ability to measure lactose at even higher concentrations (see, e.g., FIG. 11). For example, the linear response detection range of biosensing element embodied in FIG. 11 was extended into this higher concentration regime as a direct result of the increased mass transfer resistance of the less porous diffusion layer.

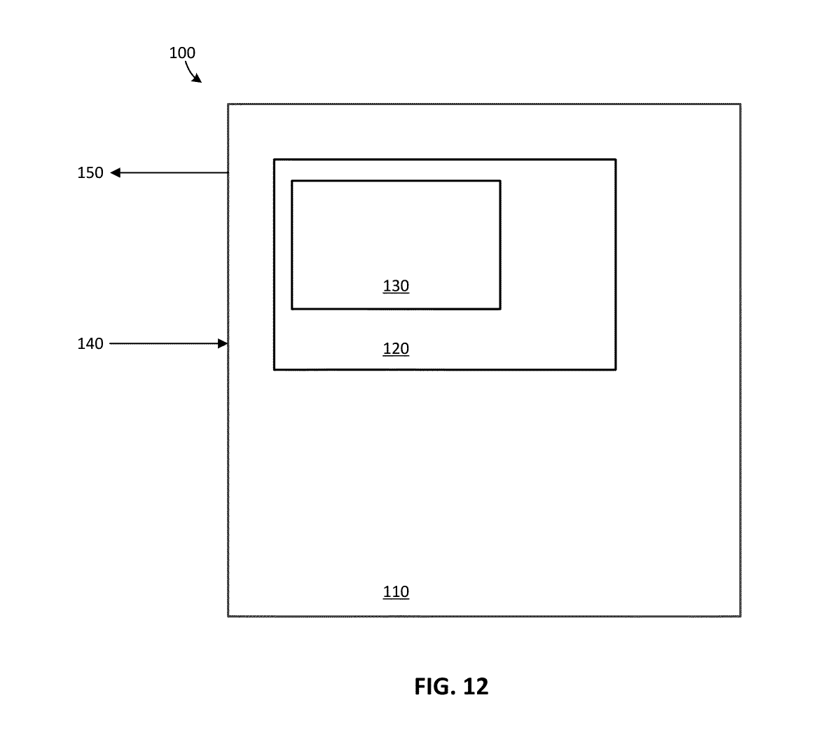

FIG. 12 shows one exemplary embodiment of a system 100 that is used to provide the appropriate design parameters for constructing biosensing elements used in biosensing systems that have a linear response in a given range of an analyte concentration in a solution. System 100 uses a computer 110 that has a microprocessor 120 that contains software 130 that processes input data 140 to provide output data 150 that contains the appropriate design parameters used for constructing biosensing elements used in biosensing systems that have a linear response in a given range of an analyte concentration in a solution. Output data 150 is displayed upon a screen or saved in a memory storage device or may be transmitted to another memory device or display device.

### Effects of Environmental Conditions

Effects of two different environmental conditions on the response characteristics of peroxide and lactose biosensing systems are summarized below.

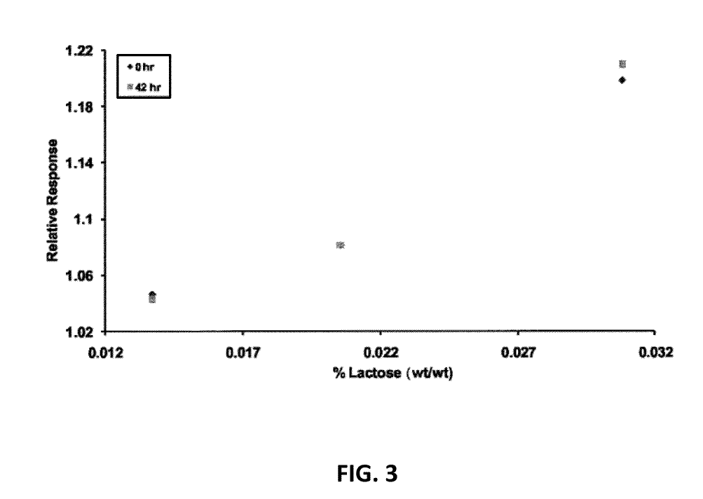

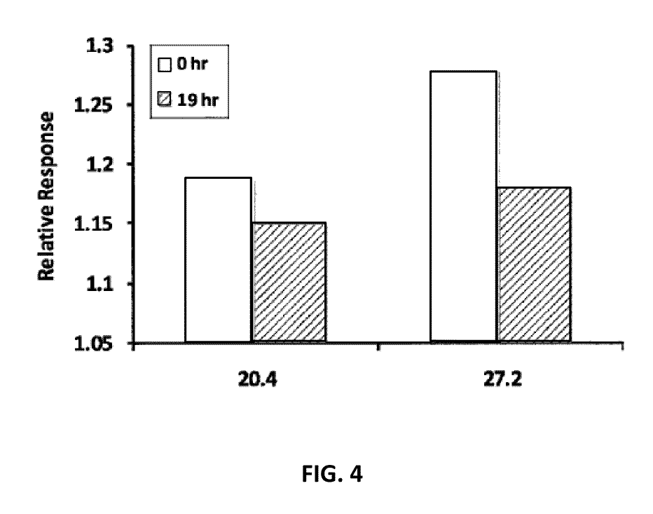

Condition 1. The first set of environmental tests involved exposing biosensing elements to a solution at pH 4.8 and 40° C. for 42 hours. The response of a lactose biosensing system at 0 and 42 h under these conditions is shown in FIG. 3. The enzyme does not lose activity under this set of conditions. Results for the same test with a H₂O₂ biosensing system are shown in FIG. 4. The enzyme in this biosensing element lost activity under this given set of environmental conditions.

In a prophetic example, an alternative to making biosensing elements that do not appreciably lose activity during a given amount of time at a given temperature, such as the parameters of condition 1, is to calibrate the biosensing system to account for loss of signal with time.

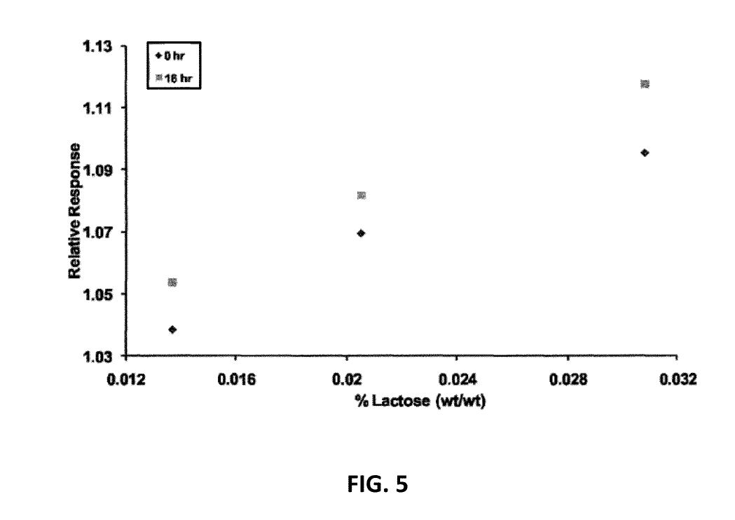

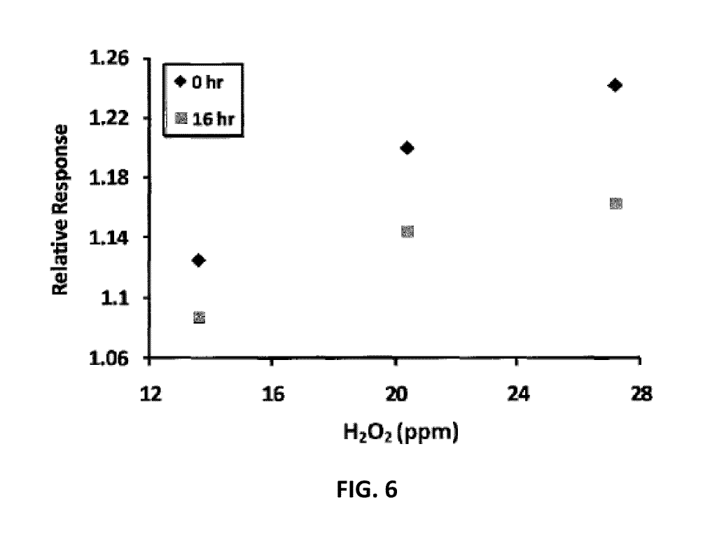

Condition 2. The second set of environmental tests involved incubating biosensing elements in a solution at pH 6.5 and 49° C. for 16 hours. Results for the lactose biosensing system are shown in FIG. 5. The stability of this biosensing element was tested over a period of 16 h. The enzyme biocomponent used in this biosensing element was stable under the given set of conditions. FIG. 6 shows a similar experiment conducted with a H₂O₂ biosensing element and, like the earlier results seen for Condition 1 using this biosensing element type, there is a decrease in enzyme biocomponent activity over time.

In a prophetic example, an alternative to making biosensing elements that do not appreciably lose activity during a given amount of time at a given temperature, such as the parameters of condition 2, is to calibrate the biosensing system to account for loss of signal with time.

Constructing the Biosensing System and/or Biosensing Element

In one embodiment, the biosensing element is constructed by putting an immobilized biocomponent within a matrix and coupling that biocomponent-containing matrix onto a transducer. In another embodiment, a biosensing system is created by bonding, affixing or otherwise causing the biocomponent to be in contact with an optode.

In one aspect, the biosensing system includes an optode having an optical fiber with a first tip (also referred to as the distal tip), and a second tip (also referred to as the proximal end). The first tip can be covered by a luminescent transducer layer, and the luminescent transducer layer can be covered by a biocomponent layer, which is located distal to the transducer layer. The biocomponent layer can be covered by a diffusion layer or barrier (e.g., porous membrane) that is distal to the biocomponent layer. Additionally, the second tip can be coupled to a photon-detection device, and the photon-detection device can coupled to a signal processing system. The biosensing system biocomponent (e.g., purified enzymes) can be immobilized in the biocomponent layer using a matrix that has been treated with cross-linking agents. Any suitable cross-linking agents can be used, including but not limited to glutaraldehyde, hexamethylene diisocyanate and 1,5-dinitro-2,4-difluorobenzene, glutaraldehyde, polyethyleneimine, hexamethylenediamine and formaldehyde. The biosensor luminescent transducer layer can be located in the first tip of the optical fiber, proximal to the purified enzymes immobilized in the matrix of the biocomponent layer. The transducer layer can be constructed of various materials, including but not limited to, cellulose, cellulose derivatives, silica, glass, dextran, starch, agarose, porous silica, chitin and chitosan.

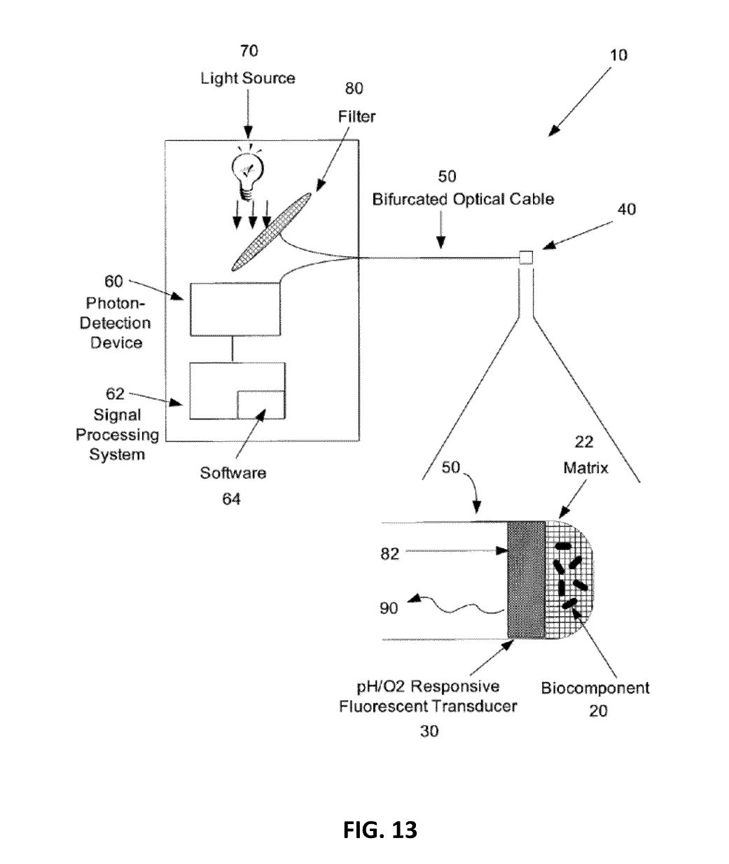

An embodiment of biosensing system of the present disclosure is depicted in FIG. 13. FIG. 13 depicts a biosensing system 10. Biosensing system 10 includes a biocomponent 20 that is displaced within a matrix 22. Matrix 22 is in direct contact with a transducer 30. Transducer 30 is in direct contact with an end of a bifurcated optical cable 50. Biocomponent 20 and transducer 30 comprise a biosensing element 40. Bifurcated optical cable 50 transmits light from a light source 70 through a filter 80. The light that is transmitted through filter 80 is transmitted through bifurcated optical cable 50 at a first light wavelength 82. Transducer 30 interacts with first light wavelength 82 and luminesces at a second light wavelength 90. Second light wavelength 90 is transmitted through bifurcated optical cable 50 and is detected by a photon-detection device 60 that produces a signal that is sent to a signal processing system 62. Signal processing system 62 contains software 64 that uses an algorithm for determining the concentration of an analyte in a solution based on the luminescence of transducer 30 at second wavelength 90.

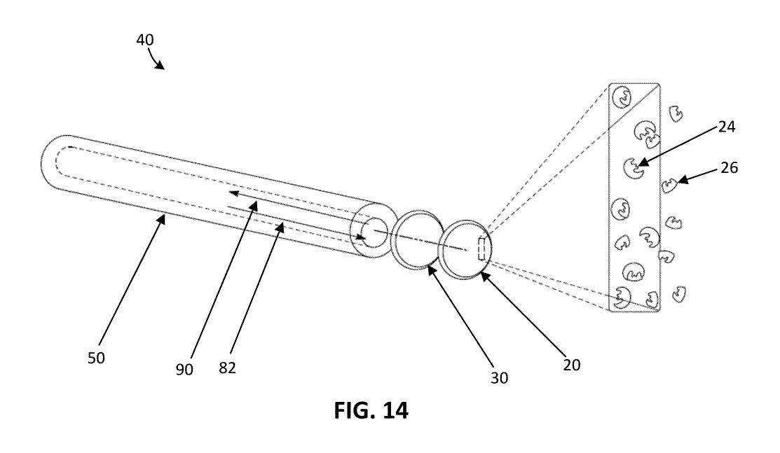

In one embodiment, the biocomponent 20 at the distal tip (i.e., first tip) of biosensing element 40 includes purified or substantially purified enzymes 24 immobilized in a matrix in the biocomponent 20 that interact or associate with one or more analytes 26 (FIG. 14). Separate layers comprising the transducer 30 and biocomponent 20 may be constructed of a matrix that includes a plurality of enzymes 24 to which analytes 26 may bind. For example, when an analyte 26, such as alcohol or glucose, diffuse into the matrix in which biocomponent 20 is immobilized, enzymes 24, such as oxidases, bind to and interact with the analyte 26, and an enzyme-catalyzed reaction occurs in which oxygen is consumed. This reaction alters the characteristic fluorescence lifetime of an immobilized fluorophore in the transducer 30 and is proportional to the concentration of the analyte 26. For example, oxygen can be consumed in the reaction catalyzed by one or more oxidases, such as alcohol oxidase or glucose oxidase, which acts as a transduction pathway to measure the alcohol concentration using a fluorescent dye that is sensitive to oxygen.

Method of Using the Biosensing System and/or Biosensing Element

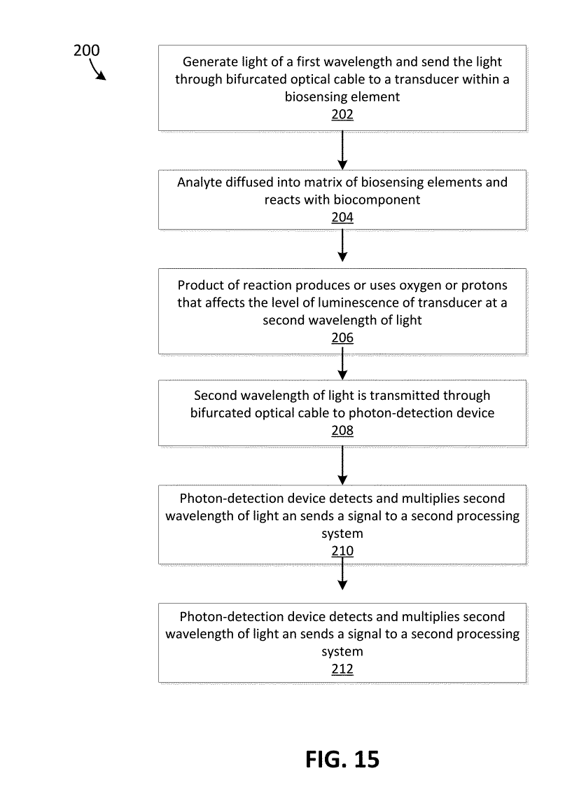

FIG. 15 shows one exemplary method 200 for using a biosensing system to measure the concentration of an analyte in a solution. In step 202, method 200 is implemented by generating light of a first wavelength 82 by light source 70 as it passes through filter 80 and travels down bifurcated optical cable 50 to interact with transducer 30 of biosensing element 40. In step 204, an analyte diffuses into matrix 22 and reacts with biocomponent 20. In step 206, the product of the reaction of the analyte with biocomponent 20 produces or uses oxygen and/or hydrogen ions that interact with transducer 30 to affect the amount of fluorescence at a second light wavelength 90 of transducer 30. In step 208, the second light wavelength 90 is transmitted through bifurcated optical cable 50 and detected by photon-detection device 60. In step 210, photon-detection device 60 detects and multiplies the signal of second light wavelength 90 and sends a signal to signal processing system 62. In step 212, signal processing system 62 has software 64 that uses an algorithm that transforms the signal from photon-detection device 60 into an output that can be read as a numerical representation of the concentration of the analyte.

### Immobilization of the Biocomponent

In order to make a biosensing system and/or biosensing element, the biocomponent needs to be sufficiently bound to or in contact with the transducer. This can be achieved by immobilizing the biocomponent on the transducer. The viability of a biosensing system and/or biosensing element depends on the processing and type of material used for immobilizing the biocomponent. The material used for immobilizing the biocomponent may be referred to as a matrix, matrix material or as an immobilizing material.

Biocomponents may be very sensitive to the immobilizing process and the material that is used for immobilization. The immobilization process should not damage the biocomponent. The pH, ionic-strength, and any other latent chemistries of the matrix should be compatible with the biocomponent. The reactants and products of the biocomponent should not affect the material used for immobilization. The biocomponent should be effectively immobilized and there should not be any leakage of the biocomponent from the matrix during the active lifetime of the biosensing system and/or biosensing element. The immobilization material should be non-toxic and non-polluting. The material should have proper permeability to allow sufficient diffusion of substrates, products and gases. The matrix material should allow for sufficient cell activity and cell density. The immobilization material should protect the biocomponent from biotic and abiotic environmental stresses that would lower biocomponent activity or lifetime.

### Techniques of Immobilization

In one embodiment, adsorption is used to immobilize the biocomponent. Many substances adsorb enzymes, cells, microorganisms and other biocomponents on their surfaces, e.g., alumina, charcoal, clay, cellulose, kaolin, silica gel and collagen. Adsorption can be classified as physical adsorption (physisorption) and chemical adsorption (chemisorption). Physisorption is usually weak and occurs via the formation of van der Waals bonds or hydrogen bonds between the substrate and the enzyme molecules. Chemisorption is much stronger and involves the formation of covalent bonds. Adsorption of the biocomponent may be specific through the interaction of some moiety, link or other reactive component of the biocomponent or may be non-specific.

In another embodiment, microencapsulation is used to immobilize the biocomponent. In this method, a thin microporous semipermeable membrane is used to surround the biocomponent. Because of the proximity between the biocomponent and the transducer and the very small thickness of the membrane, the biosensing element response is fast and accurate. In one embodiment the biocomponent is bonded to the sensor via molecules that conduct electrons, such as polypyrrole. The membrane used for microencapsulation may also serve additional functions such as selective ion permeability, enhanced electrochemical conductivity or mediation of electron transfer processes. Examples of membranes that may be used for microencapsulation immobilization of biocomponents are cellulose acetate, polycarbonate, collage, acrylate copolymers, poly(ethylene glycol) and polytetrafluoroethylene (PTFE). Additional materials that may be used are agarose, and alginate and polylysine, which together form an alginate-polylysine-alginate microcapsule.

In another embodiment, entrapment is used to immobilize the biocomponent. In this method cells are physically constrained (entrapped) to stay inside a three-dimensional matrix. The materials used for entrapment allow for uniform cell distribution, biocompatibility and good transport of substrates and products. Both natural and synthetic materials (like alginate, agarose and collagen) may be used for entrapment.

In another embodiment, hydrogels are used to immobilize the biocomponent. Hydrogels provide a hydrophilic environment for the biocomponent and they require only mild conditions to polymerize. Hydrogels are capable of absorbing large quantities of water which can facilitate reactions such as hydrolysis. Both natural and synthetic hydrogels may be used such as algal polysaccharides, agar, agarose, alginate, and carrageenan, polyacrylamide, polystyrene and polyurethane.

Alginate, a hydrogel, provides a good, biocompatible microenvironment for the biocomponent with gentle encapsulation process. It is a naturally occurring linear polymer composed of β-(1,4)-linked D-mannuronic acid and a-(1,4)-L-guluronic acid monomers. Commercially, alginate is obtained from kelp, but bacteria such as Azotobacter vinelandii, several Pseudomonas species and various algae also produce it. When alginate is exposed to Ca²⁺ ions, a cross-linking network is formed by the bonding of Ca²⁺ ions and polyguluronic portions of the polymer strand by a process known as ionic gelation. The gelation process is temperature-independent. Complete gelling time without biocomponents may be from about 1 minute to greater than about 30 minutes. Gelling time usually increases with an increase in biocomponent density and decreases with an increase in CaCl₂ concentration.

In another embodiment, sol-gels may be used to entrap biocomponents into silicate networks. Sol-gels, which do not require the use of cross-linking agents to form matrices, may require milder polymerization processes and create matrices that exhibit good mass transport and molecular access properties particularly for electrochemical and optical transduction modes. A sol-gel is composed of silicates and can be used to entrap the detection enzyme and retain it on the tip of a sensor. Since silicate sol-gels are often brittle and may crack, a sol-gel fabrication protocol can be used in which a polymer such as polyvinyl alcohol is blended with the sol-gel as it hardens, producing a matrix that is more pliable and less likely to crack than a simple sol-gel.

In another embodiment, a bovine serum albumin, or BSA, matrix can be used to immobilize enzymes. BSA is a protein that is readily available in a purified form. The matrix is formed by mixing the detection enzyme with BSA and then using glutaraldehyde to cross-link both of these proteins together, forming an insoluble matrix in which the detection enzyme is entrapped. In other embodiments, lysozyme can be used instead of BSA to immobilize enzymes within a cross-linked matrix.

In another embodiment, cross-linking is used to immobilize the biocomponent. Cross-linking chemically bonds the biocomponent to solid supports or to other supporting materials such as a gel. Bifunctional agents such as glutaraldehyde, hexamethylene diisocyanate and 1,5-dinitro-2,4-difluorobenzene may be used to bind the biocomponent to the solid support. Cross-linking produces long-term stability under more strenuous experimental conditions, such as exposure to flowing samples, stirring, washing, etc.

In another embodiment, covalent bonding is used to immobilize the biocomponent. Covalent bonding uses a particular group present in the biocomponent, which is not involved in catalytic action, and attaches it to the support matrix (transducer or membrane) through a covalent bond. The radicals that take part in this reaction are generally nucleophilic in nature (e.g., —NH₂, —COOH, —OH, —SH and imidazole groups).

### Stabilization

Generally, it has been challenging to produce biosensing systems that are stable and long-lived. However, biosensing systems and biosensing elements of the present disclosure are stable and long-lived, can stand prolonged storage and can also perform well in use for extended periods. Biocomponents may be stabilized through various means, depending upon the type of biocomponent and transducer used.

In one embodiment, the biocomponent may be stabilized through molecular modification. Molecular modification improves the stability of enzymes, and other biocomponents, through changing certain amino acids or nucleotides in the peptide or nucleic acid sequence, respectively. Molecular modifications may increase the temperature stability of various enzymes by modifying the amino acids at the catalytically active enzyme reaction site, through site-directed mutagenesis.

Another method for improving the stability of biocomponents, such as enzymes, is through glycosylation. Since glycosylated proteins are very stable, grafting or otherwise bonding polysaccharides or short chains of sugar molecules onto protein molecules usually improves the stability of the biocomponent.

In one embodiment, the biocomponent may be stabilized through cross-linking Cross-linking of the biocomponent may occur through covalent bonding, entrapment, encapsulation and other immobilization techniques or processes. These immobilization processes can improve enzyme stability by reducing the biocomponent's mobility and thereby reducing degradation of its three-dimensional structure. In addition, cross-linking prevents the loss of biocomponents from their immobilized matrix. Using the entrapment method discussed above, the loss of biocomponents may further be reduced by the addition of certain gel-hardening agents such as glutaraldehyde, polyethyleneimine, hexamethylenediamine and formaldehyde.

In another embodiment for stabilizing the biocomponent, freeze drying, also known as lyophilization, may be used. Freeze drying is a method for long-term preservation of microorganisms and enzymes. It involves removal of water from frozen bacterial suspensions by sublimation under reduced pressure. The lyophilization is performed in the presence of cryoprotective agents such as glycerol and DMSO which reduce the damage caused during freezing and during thawing. Lyophilized biocomponents, for example dried cells, are stable to degradation by keeping the lyophilized biocomponents below 4° C., and away from oxygen, moisture and light. Even after prolonged periods of storage, such as about 10 years, lyophilized biocomponents may then be rehydrated and restored to an active state. Two examples of lyophilizing of biocomponents include centrifugal freeze-drying and prefreezing.

In another embodiment, the biocomponents may be stabilized through heat shocking Heat shocking involves heating vacuum-dried cells at a high temperature (about 300° C. for example) for a very short time (about 2-3 minutes for example). With the proper combination of temperature and heating time, biocomponents such as whole cells and microorganisms can be killed but still retain an active enzyme system that may be used to detect a compound of interest. These dead cells and microorganisms can be kept for a long time away from moisture without any requirement of nutrients.

In another embodiment, the addition of carbohydrates and other polymers will stabilize the biocomponents. Carbohydrates used to stabilize biocomponents include polyalcohols and various sugars such as trehalose, maltose, lactose, sucrose, glucose and galactose, for example. This stabilization may occur due to the interaction of polyhydroxyl moieties from the polyalcohols and/or sugars with water with the biocomponents, thus increasing hydrophobic interactions and keeping the biocomponents in a stable conformation.

In an additional embodiment, stabilization of the biocomponents may occur through freezing the biocomponents. When a biocomponent is frozen, the metabolic activities may be reduced considerably. Storage of the biosensing elements at temperatures wherein the biocomponents remain frozen may increase the stability and lifetime of the biosensing system.

The disclosure will be further described in the following examples, which do not limit the scope of the disclosure described in the claims.

### Examples

### pH Optode Construction

Plastic clad fiber optic cables with core diameter of 1 mm and length of 6-8 inches were used to make biosensing elements for use in biosensing systems. The first 1.5 to 3 mm of cladding was removed from both ends of these cables using wire strippers, taking care not to scratch the sides of the fiber. Each surface was polished in a figure eight pattern using polishing glass, fine grit papers and a polishing disc which held the optical fiber perpendicular to the polishing surface. After polishing, each end was cleaned with isopropyl alcohol and examined at 100× magnification under a microscope to ensure that there were no scratches through the core and no chips in the edges that extend into the core of the fiber. The smooth surface of the fiber-end was important for producing a stable response and for reducing signal losses due to refraction of light. Around 5 mm of cladding was removed again from one of the ends of the fiber in order to insert a connector ferrule which connects each sensor to the 1 m long optical fiber. From the sensing end around 1 mm of the cladding was removed. Each of these cables was fit with a gasket and a cap to fit a 2 mL glass vial at the sensing end.

The pH optode was formed using a modified immobilization procedure by affixing a pH-sensitive fluorescent dye to the end of the fiber optic cable. At first, 0.5 g of cyanuric chloride was dissolved in 20 mL of acetone. To this solution, 1.0 g of polyvinyl alcohol (PVA, MW=10,000) and 10 mL of deionized water (dH₂O) were added. After mixing for 17 minutes at room temperature, this solution was filtered and the resultant filtrate was washed with a mixture of water/acetone (1:2). This filtrate was then added in a solution containing 100 mg of fluoresceinamine in 10 mL acetone. The mixture was allowed to react for 35 minutes, then was filtered, washed with small amount of acetone (˜10 mL) and subsequently dried.

In order to make the hydrogel, 5 μL of 6M HCl (acts as catalyst), 5 μL of 5% (v/v) solution of glutaraldehyde (Grade 1: 50% solution) and 25 μL of 5% (w/v) of PVA/fluoresceinamine dye in dH₂O were mixed together. One drop of this mixture was added to the tip of the optical fiber using a 100 μL pipette and allowed to polymerize for ˜30 seconds. Prior to the transfer step, the fiber optic end was cleaned by exposure to 2 M HCl followed by washing with water and then drying. This was important as the hydrogel adhered best to a cleaned surface. After the tip was coated, it was then stored in 0.1 M Na₂HPO₄ (Sigma Chemicals, 99% purity) at room temperature.

In order to test the performance of each pH optode, these probes were connected to the detector system and the pH optode was allowed to reach equilibrium (>99% of steady state value) in a phosphate buffer solution at a pH of 7. Once it reached equilibrium (allowed to stay at equilibrium for couple of minutes), it was then transferred to another solution phosphate buffer having a pH of 6.9 and allowed to reach a new equilibrium (response time of about 3 to about 5 minutes). These values of pH of the buffer solution were chosen because they lie in the groundwater pH range in which biosensing element would be finally tested. These readings were taken at 800 V and the PMT amplifier adjusted to obtain a signal in the linear response range. Two criteria used in deciding whether the biosensing element is good enough or not were the magnitude of the change in equilibrium value (more is better) and stability in the biosensing element response.

### Preparation of Biocomponent

### Cell Cultures

Cells may be grown and isolated by methods well known in the art. In order to make a biosensing system, cells were immobilized using the entrapment method. The cells used for immobilization had been stored at 4° C. in a phosphate-buffered saline solution. This cell suspension was centrifuged at 15000×g for 2 minutes. The cell pellet was then washed with saline (9 g/L of NaOH [pH 7.1]) and again centrifuged. This cycle was repeated three times. Then a 4% (w/v) aqueous solution of Na-alginate was added at a ratio of about 1.0 to about 1.2 g/g. Next, the sides of the pH optode were carefully rinsed and wiped to remove any traces of phosphate, which inhibits gelation. The cell-alginate mixture was stirred well with a pipette tip and a small drop of gel was carefully deposited on the tip of the pH optode. The tip was now dipped into an ice-cold solution of 7% (w/v) of CaCl₂ 2H₂O for 15 minutes. When exposed to Ca²⁺ ions, a cross-linking network was formed by the bonding of Ca²⁺ ions and polyguluronic portions of the polymer strand by a process known as ionic gelation. Gelling time increases with increase in cell density and decreases with increase in CaCl₂ concentration. After immobilization, the diameter of the biosensing element on the tip was about 2 mm. Once the biosensing element was made, it was stored in the measurement solution (NaOH solution [pH 7.0] in which all the readings were taken).

Although the pH-sensitive dye layer is quite stable physically and does not easily fall off from the optode tip, it is advisable not to touch the optode surface with a pipette tip. Also, in order to have a stable response, it was important that no bubbles were present inside the bead after immobilization.

### Preparation of Biosensing Element Using Dry-Heated Cells

In order to prepare dry heated cells, cells stored at 4° C. in phosphate-buffered saline solution were centrifuged at 15,000×g for 3 minutes and were washed twice with distilled water. These cells were suspended in a small quantity of water (3 mL of stored cell suspension were washed and then suspended in 0.5 mL of water). This suspension was put in a 10-mL beaker and water was completely removed by vacuum drying at 35° C. It took about an hour to dry these cells. The dried cells were then scratched off from the surface of beaker using a spatula. The beaker was then covered with aluminum foil and left in the oven at a constant temperature of 270° C. and for a given period of time (30 sec, 60 sec, etc.). These dry heated cells looked like a highly porous solid and had a light orange color. These dry-heated cells (˜0.003-0.004 g) were also immobilized using the same entrapment method. However it was found that when these cells were directly mixed with 4% (w/v) of alginate, there were a lot of small bubbles in the cell-alginate mixture. Since it was important to eliminate these bubbles in order to obtain a stable response, these cells were first suspended in 10 μL of NaOH (pH 7.0) in a 1.5 mL-vial and then 8% (w/v) of alginate was added to it (from about 0.3 to about 0.5 μg of dry wt. of cells to wt. of alginate). This mixture was used to make the biosensing element.

### Preparation of Biosensing Element Using Chloramphenicol-Treated Cells

Cells stored at 4° C. in phosphate-buffered saline were centrifuged at 15,000×g for 2 minutes and the pellet was then washed twice with saline (9 g/L of NaCl [pH 7.1]) containing 50 μg/mL of chloramphenicol. Next, sodium alginate (4% w/v in water) containing either 50 or 200 μg/mL of chloramphenicol was added and mixed well with the cell pellet. This cell and alginate mixture was kept for 5 minutes at room temperature before it was used to make the biosensing element.

### Preparation of Biosensing Element Using Protease Inhibitor-Treated Cells

Cells stored at 4° C. in phosphate-buffered saline were centrifuged at 15,000×g for 2 minutes and the pellet was then washed twice with saline (9 g/L of NaCl [pH 7.1]) containing 5 μL of protease inhibitor cocktail in 1 mL of saline solution. This cocktail was prepared by adding 215 mg of lyophilized protease inhibitor in a solution containing 1 mL of DMSO (Dimethyl sulfoxide) and 4 mL of deionized water. The cocktail had a broad specificity for the inhibition of serine, cysteine, aspartic and metalloproteases, and aminopeptidases. It was stored at −20° C. in the freezer. These cells were then mixed with Na-alginate solution (4% w/v) containing 200 μL of cocktail per mL of alginate solution. The cell-alginate mixture was left for about 5 minutes at room temperature before it was used for making the biosensing element. The ratio of the weight of wet cells to the weight of alginate used in the experiment was 0.72 g/g.

Preparation of Biosensing Element with a Poly-L-Lysine Coating

The alginate bead was coated with poly-L-lysine (PLL) by preparing the tip of a biosensing element with a biocomponent as described above. The Ca-alginate bead on the biosensing element tip was then washed twice with saline solution (9 g/L of NaCl in water). Then the tip of the biosensing element was immersed in 10 mL of 0.4% (w/v) of poly-L-lysine.HCl solution, stored at 4° C. inside the refrigerator) in saline for 30 minutes at 30° C.

### Oxygen Optode Construction

In one embodiment, the transducer used in a biosensing system is an oxygen optode. An oxygen optode is a sensor based on optical measurement of the oxygen concentration. In one embodiment, a chemical film is glued to the tip of an optical cable and the fluorescence properties of this film depend on the oxygen concentration. Fluorescence is at a maximum when there is no oxygen present. When an O₂ molecule collides with the film, it quenches the photoluminescence. In a given oxygen concentration, there will be a specific number of O₂ molecules colliding with the film at any given time, and the fluorescence properties will be stable.

In one example, a biosensing system for measuring the concentration of oxygen consisted of a layer of immobilized whole cells over an oxygen optode, which was constructed from a 25-cm section of PMMA optical fiber terminated with a straight tip connector. The fiber jacket was detached 1 mm from the end (non-connector terminated) and then polished with 2000-grit and 3 μm polishing film (part of a fiber optic tool kit, IF-TK4-RP2, Industrial Fiber Optics) to minimize potential signal loss due to scattering. One mg of the oxygen-sensitive phosphorophore RuDPP, which is classified as a phosphorophore since its decay lifetime is longer than typical fluorophores, was dissolved into 1 mL chloroform and mixed with 200 mg silicone gel (clear RTV silicone, Permatex, Inc.). A 1-μL aliquot of this mixture was then added to the polished fiber tip. The RuDPP gel layer was affixed to the optical fiber end as soon as the chloroform evaporated. In one prophetic example, previously stored E. coli whole cells (with different plasmids which may encode for galactosidases, lactose oxidases, carbohydrate oxidases, glucose oxidases, galactose oxidases, cellobiose dehydrogenases, and/or catalases, for example) were centrifuged and mixed with sodium alginate solution (2.5%) in a cell-to-alginate ratio (wet cell mass:alginate solution) of 1:1 w/w unless otherwise specified. In one example, purified enzymes comprising galactosidases, lactose oxidases, carbohydrate oxidases, glucose oxidases, galactose oxidases, cellobiose dehydrogenases, and/or catalases, for example, were mixed with sodium alginate solution (2.5%) in a cell-to-alginate ratio (wet cell mass:alginate solution) of 1:1 w/w unless otherwise specified. Two 1 μL aliquots of the cell-alginate mixture were placed on the tip of each oxygen optode and immobilized after immersing the optode in 0.47 M calcium chloride solution for 30 min at 0° C. All biosensing elements were stored at 0° C. in a measurement solution of 0.15 M NaCl and 0.025 M CaCl₂ at pH 7.0.

### Oxygen Optode Based Biosensing System

In one example, the oxygen optode based biosensing system instrumentation consisted of two separate optoelectronic modules: a 470-nm LED and a 450/60 nm optical bandpass filter (Chroma Technologies) as the excitation light source, and a computer-controlled Ocean Optics USB4000-FL spectrometer with 10 nm resolution for detection. The 470-nm excitation light was delivered through one leg of a bifurcated optical fiber assembly that has two 1-mm fibers side-by-side in the common end (Ocean Optics, Inc.), which was connected with the biosensing system via a straight tip connector. The phosphorescent emission light (peak at 620 nm) from the biosensing system was directed back into the detector through the other leg of the bifurcated optical fiber and measured by the spectrometer (sensitivity of approximately 60 photons/count at 600 nm). The spectrometer output from 615 nm to 625 nm was integrated over 200 ms and five such values were averaged to yield one measurement value per second. The change in the intensity or change in the lifetime decay of the emission light over time correlates to the oxygen concentration change at the RuDPP layer of the biosensing element.

### Carbohydrate Analytes

In some embodiments, the biosensing systems of the present disclosure can also be designed to detect and monitor various other carbohydrates and/or carbohydrate-based analytes, including but are not limited to, glucose, sucrose, galactose, and xylose, and analytes incorporating these and other carbohydrates (i.e., carbohydrate-based). As with lactose biosensing systems, biosensing systems designed to detect and monitor other carbohydrate analytes may comprise a biocomponent, and a transducer, and a photon-detection device, and a signal processing system. The biosensing system biocomponent can be glucose oxidase, lactose oxidase, pyranose oxidase, as well as other oxidases, as is apparent to one of ordinary skill in the art based on the present disclosure. In one embodiment, the biosensing system biocomponent includes glucose oxidase and catalase and the transducer interacts with oxygen. In another embodiment, the biosensing system biocomponent is glucose oxidase and the transducer interacts with protons. In another embodiment, the biosensing system biocomponent is glucose oxidase and catalase and the transducer interacts with protons. In another embodiment, the biosensing system biocomponent is glucose oxidase and the transducer interacts with oxygen and protons. In another embodiment, the biosensing system biocomponent is glucose oxidase and catalase and the transducer interacts with oxygen and protons. In another embodiment, the biosensing system biocomponent is pyranose oxidase or lactose oxidase and the transducer interacts with oxygen. In another embodiment, the biosensing system biocomponent is pyranose oxidase or lactose oxidase and catalase and the transducer interacts with oxygen. In another embodiment, the biosensing system biocomponent is pyranose oxidase or lactose oxidase and the transducer interacts with protons. In another embodiment, the biosensing system biocomponent is pyranose oxidase or lactose oxidase and catalase and the transducer interacts with protons. In another embodiment, the biosensing system biocomponent is pyranose oxidase or lactose oxidase and the transducer interacts with oxygen and protons. In another embodiment, the biosensing system biocomponent is pyranose oxidase or lactose oxidase and catalase and the transducer interacts with oxygen and protons. In another embodiment, the biosensing system biocomponent is carbohydrate oxidase and the transducer interacts with oxygen. In another embodiment, the biosensing system biocomponent is carbohydrate oxidase and catalase and the transducer interacts with oxygen. In another embodiment, the biosensing system biocomponent is carbohydrate oxidase and the transducer interacts with protons. In another embodiment, the biosensing system biocomponent is carbohydrate oxidase and catalase and the transducer interacts with protons. In another embodiment, the biosensing system biocomponent is carbohydrate oxidase and the transducer interacts with oxygen and protons. In another embodiment, the biosensing system biocomponent is carbohydrate oxidase and catalase and the transducer interacts with oxygen and protons.

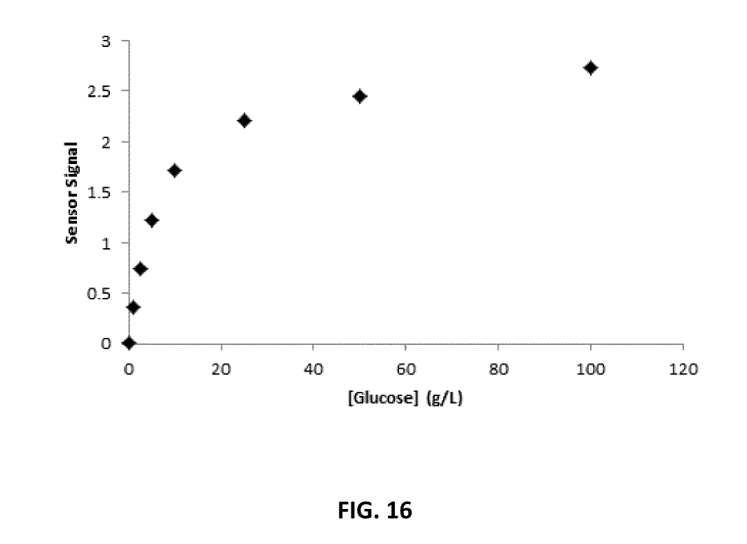

As shown in FIG. 16, a response curve was generated using a glucose biosensing system that includes a glucose sensor having a reduced amount of glucose oxidase immobilized in a cross-linked BSA matrix. The sensor was fabricated by immobilization of glucose oxidase in a glutaraldehyde crosslinked BSA matrix. Approximately 10 μL of glucose oxidase (0.125 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. These experiments were performed in stirred 200 mL beakers containing HEPES buffer at pH 7.2. Sensors were then placed in the beakers and glucose was added to the solution at the concentrations indicated in the FIG. 16. The sensors' signals were recorded after reaching a new steady state signal subsequent to addition of glucose. As shown in FIG. 16, the results of these experiments demonstrate the ability to make high range carbohydrate measurements through adding a reduced amount of oxidase enzyme to the enzymatic sensing layer.

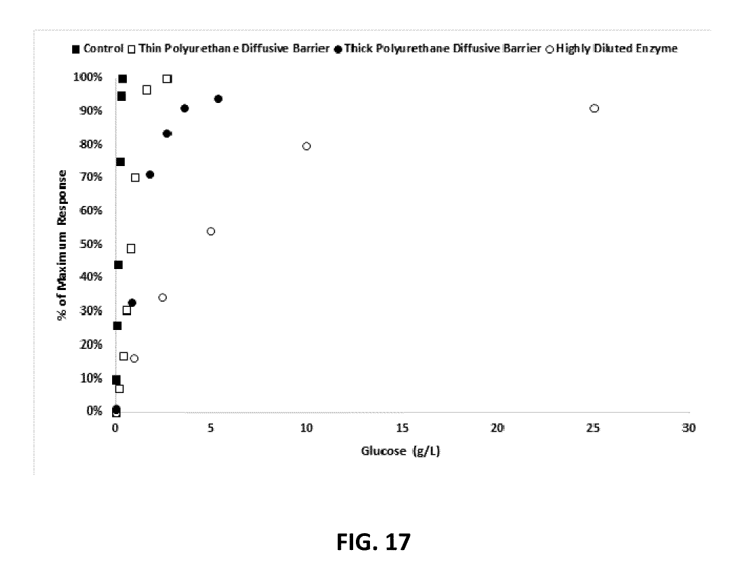

As shown in FIG. 17, response curves were generated using glucose biosensing systems that include either thick or thin polyurethane diffusion layers as well as glucose sensors having glucose oxidase immobilized in a cross-linked BSA matrix. Various analyte detection ranges (linear) were achieved by adding diffusion layer and by decreasing the amount of glucose oxidase within the layer(s) of the biocomponent. The control sensor was fabricated by immobilization of glucose oxidase in a BSA matrix crosslinked by glutaraldehyde. Approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2.

As shown in FIG. 17, thick or thin coats of polyurethane were applied on top of the glucose oxidase-BSA layer by dip coating. The solution used for the thick layer was produced by adding 5 g of polyurethane to 50 mL of toluene and stirred for 12 hours at 50° C. The relative degree of thickness and thinness can be determined experimentally and/or mathematically, as one of ordinary skill in the art would understand based on the present disclosure. This solution was then diluted 1:3 with ethanol fabricating the thin layers. Sensors were dipped into the polyurethane solutions and slowly removed to produce a thick or thin polyurethane coating based on the concentration of the solution (e.g., a more dilute solution produces a thin coating). Subsequent to dipping in the polyurethane solutions, the sensors were allowed to dry at room temperature for 10 minutes. The highly diluted enzyme sensor was then fabricated according to the parameters above. These experiments were performed in stirred 200 mL beakers containing HEPES buffer at pH 7.2. Sensors were placed in the beakers and glucose was added to the solution at the concentrations indicated in FIG. 17. The sensors' signals were recorded after reaching a new steady state signal subsequent to addition of glucose. Addition of the polyurethane diffusion layer to the sensors increases the detection range through creation of a diffusion layer or barrier, which slows diffusion of the carbohydrate into the biocomponent layer comprising the enzymes. The diffusion layer acts to reduce the diffusion rate of the carbohydrate into the biocomponent layer comprising the enzymes and shifts the linear detection range of the response curves to the left (i.e., more sensitive to lower glucose concentrations).

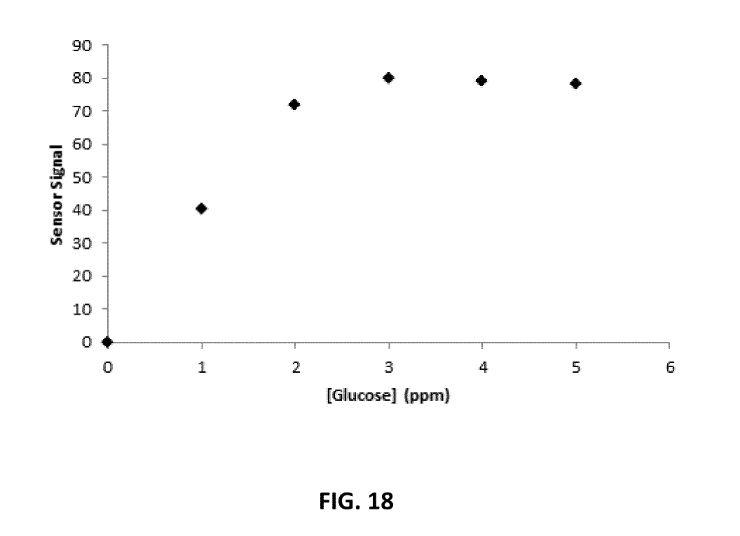

As shown in FIG. 18, a response curve was generated using a glucose biosensing system that includes a glucose sensor having glucose oxidase immobilized in a cross-linked lysozyme polymer matrix. The glucose biosensing sensor was fabricated by immobilization of glucose oxidase in a lysozyme polymer matrix crosslinked with glutaraldehyde. Approximately 40 μL of glucose oxidase (20 mg/mL) was mixed with 60 μL of lysozyme and 1 μL glycerol in DI (deionized) water. 1 μL of the resulting mixture was pipetted on top of the fluorophore layer. 1 μL of 2.5% glutaraldehyde was then pipetted on top of the glucose oxidase-lysozyme solution to initiate crosslinking. The sensor was allowed to cure for 15 minutes before being placed into HEPES buffer at pH 7.2 and stored at 4° C. until use. These experiments were performed in stirred 200 mL beakers containing HEPES buffer at pH 7.2. Sensors were placed in the beakers and glucose was added to the solution at the concentrations indicated in FIG. 18. The sensors' signals were recorded after reaching a new steady state signal subsequent to addition of glucose. As shown in FIG. 18, the results of these experiments demonstrate the efficacy of using lysozyme-immobilized glucose oxidase for measuring glucose concentrations.

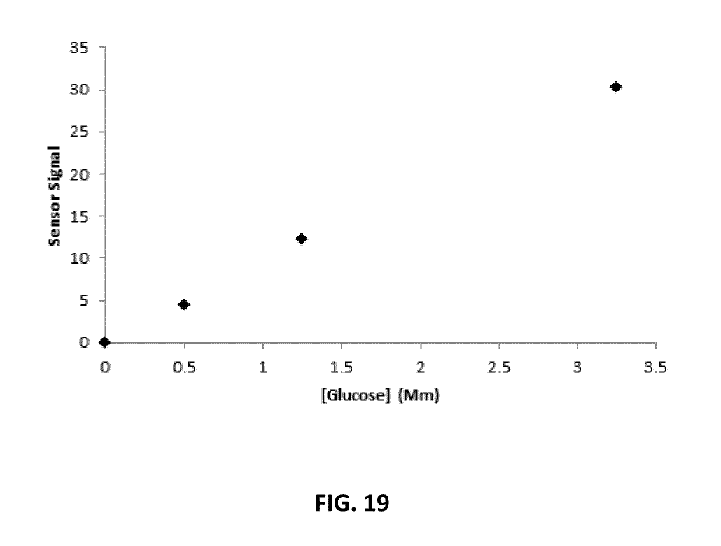

As shown in FIG. 19, a response curve was generated using a glucose biosensing system that includes a glucose sensor having glucose oxidase immobilized in an alginate polymer matrix. The glucose biosensing sensor was fabricated by immobilization of glucose oxidase in an alginate polymer by combining 1 μL of glucose oxidase-BSA solution (containing 120 μg of BSA and 10-50 μg of glucose oxidase) with 1 μL of 3, 6 or 12 percent alginate on Tau Theta oxygen-sensitive patches. Sensors were incubated for 30 seconds at room temperature, and then immersed in 0.2 M calcium chloride. Sensors were further incubated in 0.2 M calcium chloride for 2 hours at room temperature. After immobilization, sensors were stored at 4° C. in 0.1 M HEPES buffer until used. These experiment were performed in stirred 200 mL beakers containing HEPES buffer at pH 7.2. Sensors were placed in the beakers and glucose was added to the solution at the concentrations indicated in FIG. 19. The sensors' signals were recorded after reaching a new steady state signal subsequent to addition of glucose. As shown in FIG. 19, the results of these experiments demonstrate the efficacy of using alginate-immobilized glucose oxidase for measuring glucose concentrations.

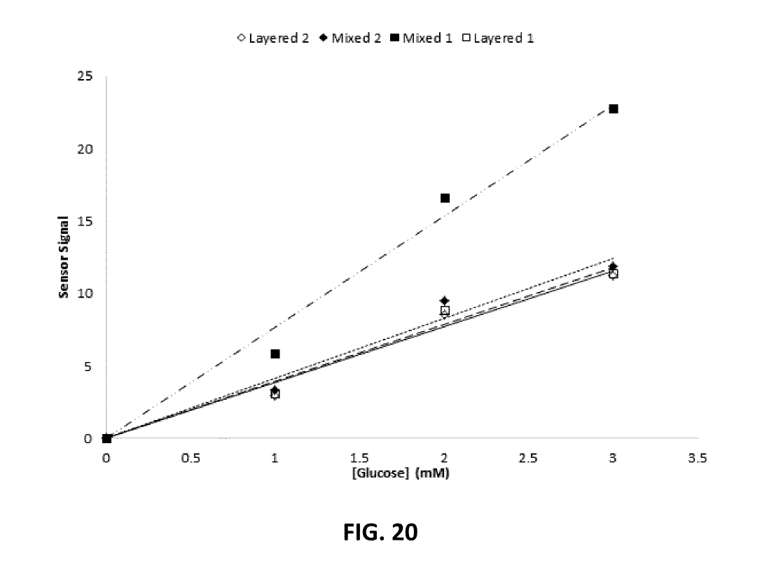

As shown in FIG. 20, response curves were generated using glucose biosensing systems that include glucose sensors having glucose oxidase and catalase either mixed in a single layer or in separate layers. For fabricating a mixed layer comprising both glucose oxidase and catalase in a single layer, approximately 40 μL of a glucose oxidase solution (25 mg/mL in HEPES buffer) was mixed with 60 μL of a 160 mg/mL BSA solution and 5 μL of glycerol (99%) (solution A). Approximately 10 μL of catalase, 90 μL BSA (56 mg/mL), and 90 μL BSA (56 mg/mL) were mixed by vortexing for 5 seconds. Solutions A and B were then combined and mixed with 100 μL of 2.5% glutaraldehyde to initiate crosslinking 0.5 μL of the resulting mixture was then pipetted on top of the flourophore layer and allowed to cure for 15 minutes. For fabricating separate layers comprising glucose oxidase and catalase, approximately 40 μL of a glucose oxidase solution (25 mg/mL in HEPES buffer) was mixed with 60 μL of a 160 mg/mL BSA solution and 5 μL of glycerol (99%). To this solution, 100 μL of 2.5% glutaraldehyde was added to initiate crosslinking Approximately 0.5 μL of the resulting solution was immediately pipetted on top of the fluorophore layer and allowed to cure for 20 min. A second solution was then prepared by mixing 10 μL of CAT, 90 μL BSA (56 mg/mL), and 90 μL BSA (56 mg/mL) by vortexing for 5 seconds. 50 μL of 2.5% glutaraldehyde was then added to the resulting solution and the mixture was vortexed for an additional 5 seconds. 0.5 μL of the resulting mixture was then pipetted on top of the glucose oxidase layer and allowed to cure for 15 minutes. As shown in FIG. 20, the results of these experiments demonstrate the feasibility of using mixed or layered sensor architectures for biosensing systems of the present disclosure.

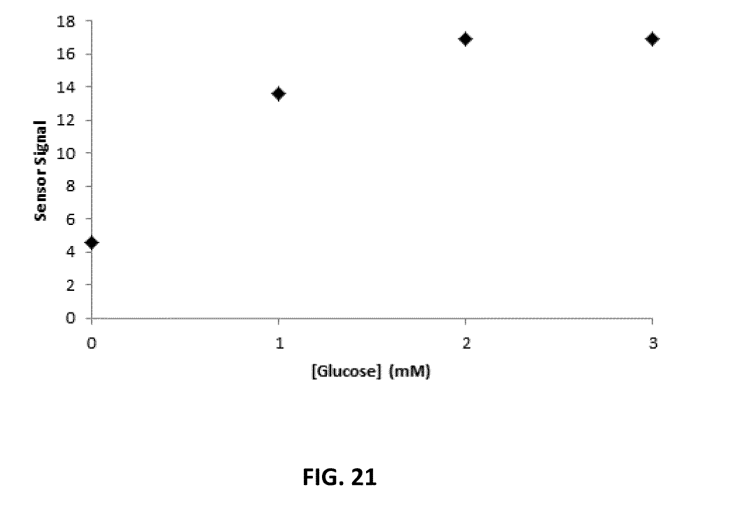

As shown in FIG. 21, a response curve was generated using a glucose biosensing system that includes a glucose sensor having glucose oxidase immobilized in a sol-gel polymer matrix. The glucose biosensor was fabricated by immobilization of glucose oxidase in a sol-gel polymer matrix. Approximately 1550 μL of tetramethyl orthosilicate (TMOS) was mixed with 450 μL DI water and 30 μL 40 mM HCl slowly for 1 hour at 4° C. (solution A). Phosphate buffer (pH 6.5, 0.1M) was mixed with 20 mg/mL glucose oxidase (25 mg/mL). Approximately 800 μL of this solution was rapidly mixed with 800 μL solution B, then 1.5 μL of the resulting mixture was pipetted on top of the fluorescent dye layer and allowed to cure for 1 minute. The resulting sensor was then placed in a vial of 0.1 M phosphate buffer (pH 6.5) and allowed to sit overnight at 4° C. before use. These experiments were performed in stirred 200 mL beakers containing HEPES buffer at pH 7.2. Sensors were placed in the beakers and glucose was added to the solution at the concentrations indicated in FIG. 21. The sensors signals were recorded after reaching a new steady state signal subsequent to addition of glucose. As shown in FIG. 21, the results of these experiments demonstrate the efficacy of using sol-gel-immobilized glucose oxidase for measuring glucose concentrations.

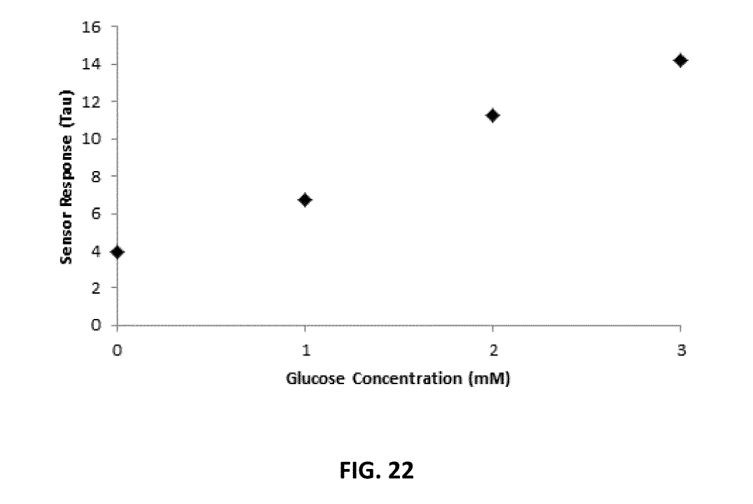

As shown in FIG. 22, a response curve was generated using a glucose biosensing system that includes a glucose sensor having lactose oxidase immobilized in a cross-linked BSA matrix. The glucose biosensor was fabricated by immobilization of lactose oxidase in a glutaraldehyde crosslinked BSA matrix. Approximately 10 μL of a dissolved lactose oxidase solution (50 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. These experiments were performed in stirred 200 mL beakers containing HEPES buffer at pH 7.2. Sensors were placed in the beakers and glucose was added to the solution at the concentrations indicated in FIG. 22. The sensors' signals were recorded after reaching a new steady state signal subsequent to addition of glucose. As shown in FIG. 22, the results of these experiments demonstrate the efficacy of using BSA immobilized lactose oxidase for measuring glucose concentrations.

In some embodiments, the biosensing systems of the present disclosure can be treated with various agents to increase or enhance the thermostability of the system. Thermostabilizing agents can be used, for example, to treat the layer of the biosensor comprising the detection enzymes (e.g., glucose oxidase). Thermostabilizing agents can include, but are not limited to β-mercaptoethanol, cysteine, dithitreitol (DTT) α-thioglycerol, and other thiol containing reducing agents and combinations thereof.

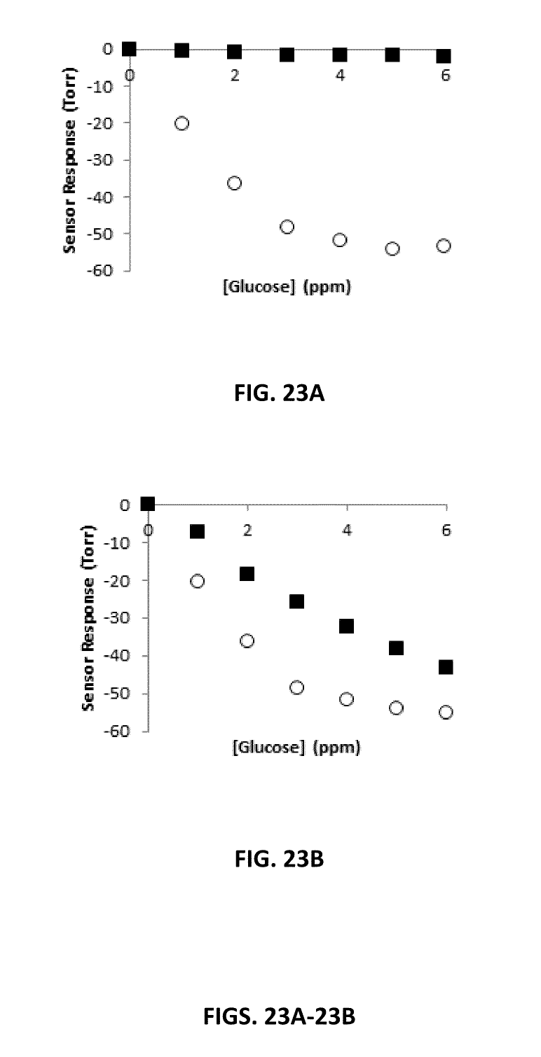

As shown in FIGS. 23A-23B, response curves were generated using glucose biosensing systems that include glucose sensors having glucose oxidase immobilized in a cross-linked BSA matrix, with (FIG. 23B) and without (FIG. 23A) mercaptoethanol treatment. As illustrated in the chemical reaction below, mercaptoethanol treatment modifies free thiols of the glucose oxidase enzyme by reducing disulfide bonds, which is manifested through an increase in the melting point of the protein. Mercaptoethanol can act to reduce the disulfide bonds in the detection enzyme such as glucose oxidase, and this reduced state may be critical for the retention of enzymatic activity.

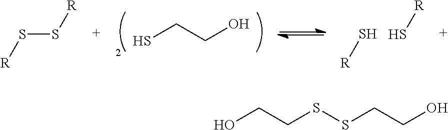

To fabricate mercaptoethanol-treated sensors, approximately 40 μL of glucose oxidase (20 mg/mL) was mixed with 60 μL of BSA (160 mg/mL) and 1 μL of glycerol in DI water. Approximately 1 μL of the resulting mixture was pipetted onto the fluorophore layer. Approximately 1 μL of 2.5% glutaraldehyde was then pipetted on top of the glucose oxidase-BSA solution to initiate crosslinking. The sensor was allowed to cure for 15 minutes, and then was placed into a solution containing 50 mM β-mercaptoethanol and 1 mM N-Ethylmaleimide and allowed to incubate for 12 hours. The sensor was then rinsed with HEPES pH 7.2 buffer 4 times for 2 hours each time and then was placed into HEPES buffer and stored at 4° C. until use. Sensors were tested for thermal stability by first measuring samples in the range of 0-6 ppm glucose. Then the sensors were incubated in water at 55° C. for 72 hours and retested in the same manner. As shown in FIGS. 23A-23B, the sensor treated with β-mercaptoethanol exhibited activity after the 55° C. incubation, while the standard sensor (no β-mercaptoethanol treatment) lost all of its activity; thus mercaptoethanol treatment has a stabilizing effect on the glucose biosensing systems.

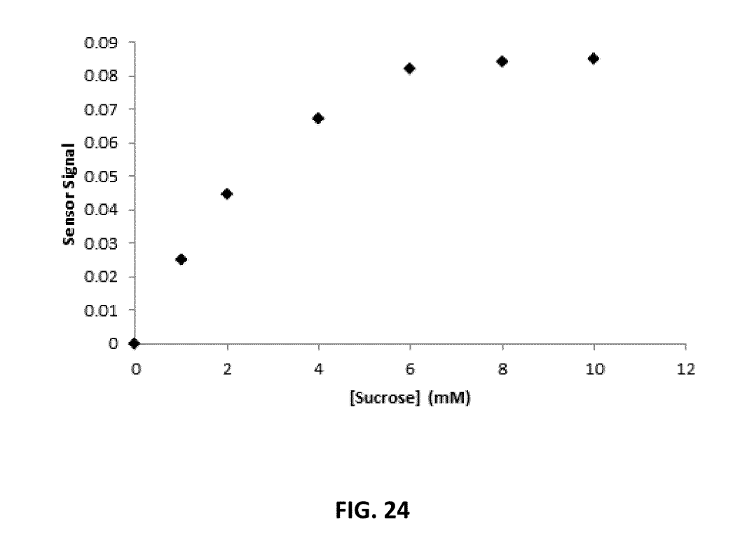

As shown in FIG. 24, a response curve was generated using a sucrose biosensing system that includes a sucrose sensor having lactose oxidase immobilized in a cross-linked BSA matrix. The sucrose biosensor was fabricated by mixing approximately 10 μL of a dissolved lactose oxidase solution (50 mg/mL) with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. Sensors were placed in the beakers and sucrose was added to the solution at the concentrations indicated in FIG. 24. The sensors; signals were recorded after reaching a new steady state signal subsequent to addition of sucrose. As shown in FIG. 24, the results of these experiments demonstrate the efficacy of using BSA-immobilized lactose oxidase for measuring sucrose concentrations.

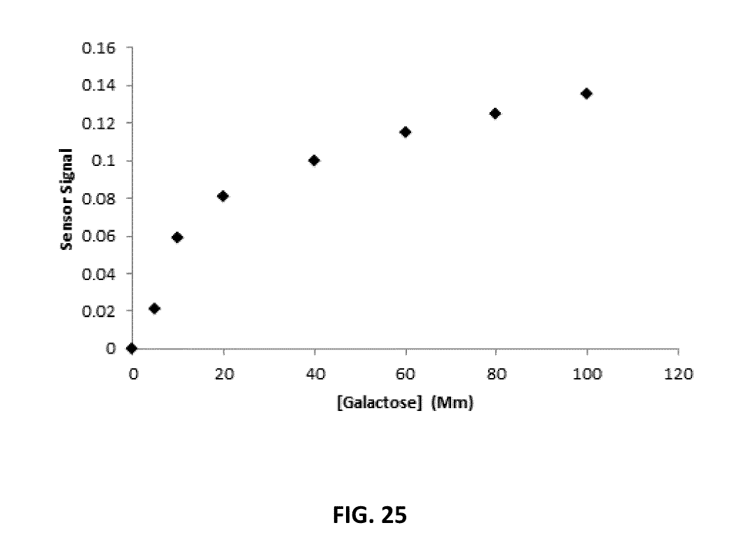

As shown in FIG. 25, a response curve was generated using a galactose biosensing system that includes a galactose sensor having lactose oxidase immobilized in a cross-linked BSA matrix. The galactose biosensor was fabricated by mixing approximately 10 μL of a dissolved lactose oxidase solution (50 mg/mL) with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. Sensors were placed in the beakers and galactose was added to the solution at the concentrations indicated in FIG. 25. The sensors' signals were recorded after reaching a new steady state signal subsequent to addition of galactose. As shown in FIG. 25, the results of these experiments demonstrate the efficacy of using BSA-immobilized lactose oxidase for measuring galactose concentrations.

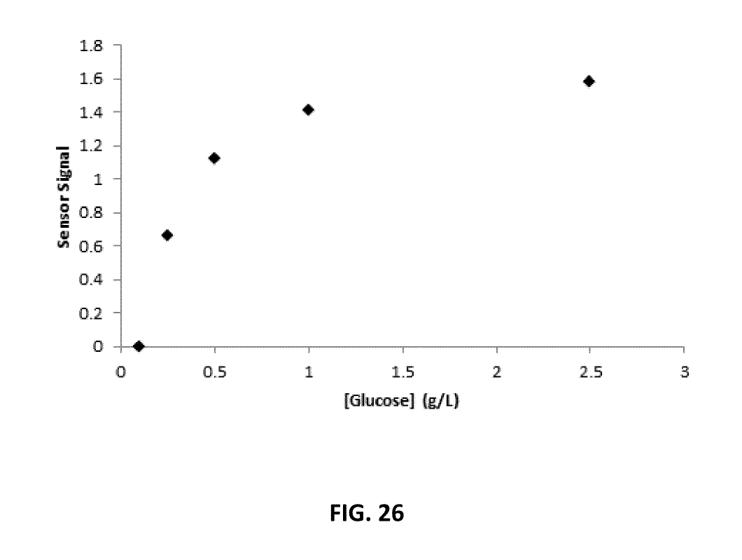

As shown in FIG. 26, a response curve was generated using a glucose biosensing system that includes a glucose sensor having pyranose oxidase immobilized in a cross-linked BSA matrix. The glucose sensor was fabricated by mixing approximately 50 μL of a dissolved pyranose oxidase solution with 50 μL of BSA (320 mg/mL in DI water), 10 μL of glycerol, and 90 μL of glutaraldehyde (2.5% in DI water). Approximately 1 μL of the resulting solution was pipetted onto the fluorophore layer and allowed to cure for 10 minutes before immersion in HEPES buffer at pH 7.2. Sensors were placed in the beakers and glucose was added to the solution at the concentrations indicated in FIG. 26. The sensors' signals were recorded after reaching a new steady state signal subsequent to addition of the glucose. As shown in FIG. 26, the results of these experiments demonstrate the efficacy of using BSA-immobilized pyranose oxidase for measuring glucose concentrations.

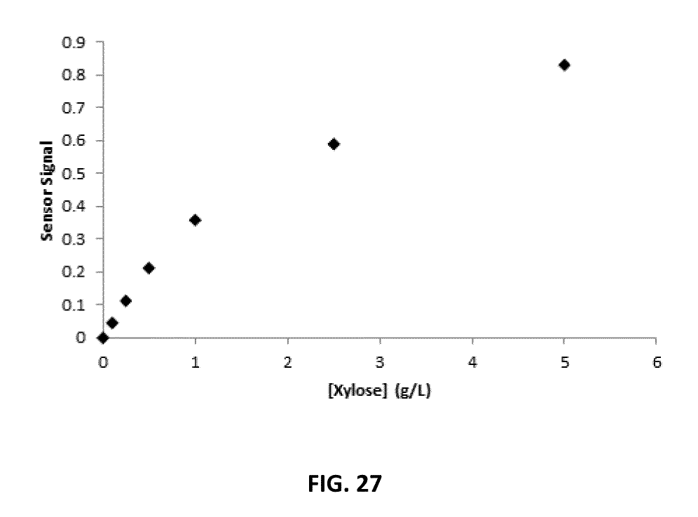

As shown in FIG. 27, a response curve was generated using a xylose biosensing system that includes a xylose sensor having pyranose oxidase immobilized in a cross-linked BSA matrix. The xylose sensor was fabricated by mixing approximately 50 μL of a dissolved pyranose oxidase solution with 50 μL of BSA (320 mg/mL in DI water), 10 μL of glycerol, and 90 μL of glutaraldehyde (2.5% in DI water). Approximately 1 μL of the resulting solution was pipetted onto the fluorophore layer and allowed to cure for 10 minutes before immersion in HEPES buffer at pH 7.2. Sensors were placed in the beakers and xylose was added to the solution at the concentrations indicated in FIG. 27. The sensors' signals were recorded after reaching a new steady state signal subsequent to addition of the xylose. As shown in FIG. 27, the results of these experiments demonstrate the efficacy of using BSA-immobilized pyranose oxidase for measuring xylose concentrations.

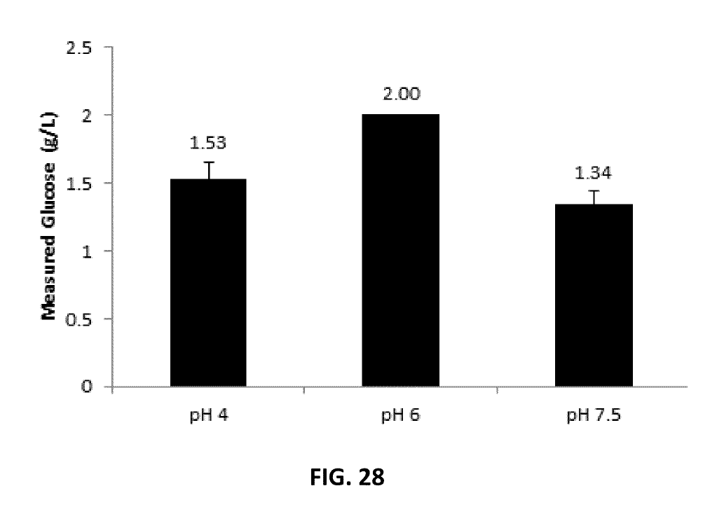

As shown in the graphical representation of FIG. 28, experiments were conducted to assess the effects of pH on glucose concentration measurements taken using glucose biosensing systems. The glucose sensors were fabricated by mixing approximately 10 μL of glucose oxidase (12.5 mg/mL) with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. Sensors were placed in the beakers and glucose was added to the solution at the concentrations indicated in FIG. 28. The sensors' signals were recorded after reaching a new steady state signal subsequent to addition of the glucose. Sensors were calibrated at pH 6 and then used to make measurements at pH 4 and 7.5. Error bars represent standard deviation for six measurements. As shown in FIG. 28, pH is a factor that affects the measurement of glucose concentrations using the glucose biosensing systems of the present disclosure.

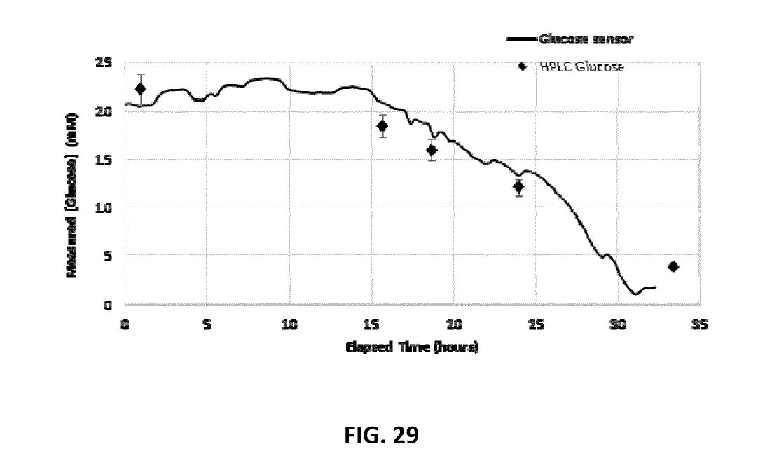

As shown in the graphical representation of FIG. 29, experiments were conducted to determine the changes in glucose concentrations during aerobic fermentation of Bacillus atrophaeus using a glucose biosensing system having a thick polyurethane diffusion layer. Aerobic fermentation of B. atrophaeus was monitored over a period of 38 hours. Glucose sensors were manufactured using glucose oxidase with a thick polyurethane diffusion layer. To fabricate the thick polyurethane layer, a solution was produced by adding 5 g of polyurethane to 50 mL of toluene and stirred for 12 hours at 50° C. This solution was then diluted 1:3 with ethanol fabricating the thin layers. Sensors were dipped into the polyurethane solutions and slowly removed to produce a thick or thin polyurethane coating based on the concentration of the solution (e.g., a more dilute solution produces a thin coating). Subsequent to dipping in the polyurethane solutions, the sensors were allowed to dry at room temperature for 10 minutes. Sensors were inserted into the 1 L fermentation vessel containing yeast extract media and 20 mM glucose. The media was inoculated with B. atrophaeus by adding 1 mL of an overnight culture. The consumption of glucose by B. atrophaeus was monitored in real-time with the glucose sensor. Grab samples were taken at the time points indicated in FIG. 29 and analyzed for glucose concentration by HPLC. As shown in FIG. 29, the results of these experiments demonstrate the efficacy of using the glucose biosensing systems of the present disclosure to monitor glucose concentrations in real-time over a significant length of time without compromising accuracy, as determined by comparisons to glucose levels measured with HPLC, which was used to generate control samples for comparison to the data generated using the sensors. These results also demonstrate that feasibility of using glucose biosensing systems in the presence of microorganisms to measure one or more bioprocesses of the microorganisms (e.g., fermentation).

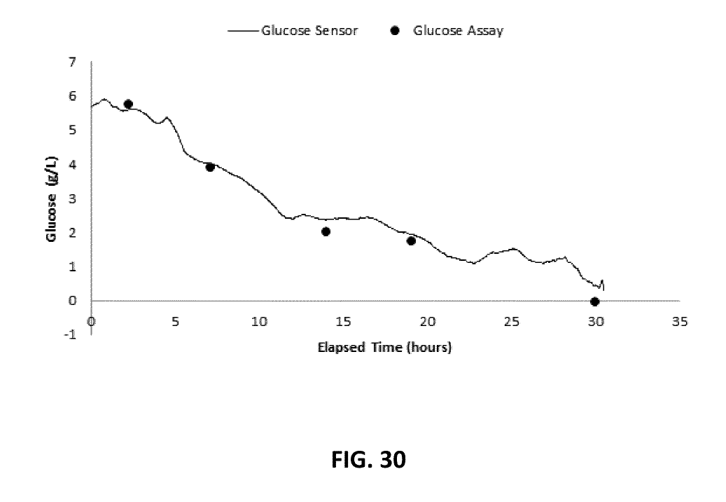

As shown in the graphical representation of FIG. 30, experiments were conducted to determine the changes in glucose concentrations during aerobic fermentation of Pichia stipitis determined using a glucose biosensing system having a thick polyurethane diffusion layer. Aerobic fermentation of P. stipitis was monitored over a period of 38 hours. Glucose sensors were manufactured using glucose oxidase with a thick polyurethane diffusion layer. To fabricate the thick polyurethane layer, a solution was produced by adding 5 g of polyurethane to 50 mL of toluene and stirred for 12 hours at 50° C. This solution was then diluted 1:3 with ethanol to fabricate the thin layers. Sensors were dipped into the thick or thin polyurethane solutions and slowly removed to produce a polyurethane coating. Subsequent to dipping in the polyurethane solutions, the sensors were allowed to dry at room temperature for 10 minutes. Sensors were inserted into the 1 L fermentation vessel containing yeast extract medium and 6 g/L glucose. The media was inoculated with P. stipitis by adding 1 mL of an overnight culture. The consumption of glucose by P. stipitis was monitored in real-time with the glucose sensor. Grab samples were taken at the time points indicated in FIG. 30 and analyzed for glucose concentration by colorimetric glucose assay. As shown in FIG. 30, the results of these experiments demonstrate the efficacy of using the glucose biosensing systems of the present disclosure to monitor glucose concentrations in real-time over a significant length of time without compromising accuracy, as determined by comparisons to glucose levels measured with colorimetric glucose assays, which were used to generate control samples for comparison to the data generated using the sensors. These results also demonstrate that feasibility of using glucose biosensing systems in the presence of microorganisms to measure one or more bioprocesses of the microorganisms (e.g., fermentation).

In some embodiments, oxidase-based biosensors, such as those designed to detect glucose, can be used to monitor analyte concentrations during cultivations of microorganisms, as demonstrated in FIGS. 29-30 above. A sensor that provides real-time, continuous data corresponding to the changing concentration of glucose or other analytes can be used to monitor and control bioprocesses. For example, a common strategy for increasing the productivity of a cultivation is to operate in fed-batch mode. This involves starting in batch mode (i.e., all nutrients are added at the beginning and this solution/mixture is inoculated) and then adding a concentrated solution of glucose and/or other nutrients to the culture. The concentrated solution is then added at a rate that maintains the glucose concentration at a desired level, even while the microorganisms are consuming the glucose. The continuous, real-time data corresponding to glucose concentrations in the solution acquired by the glucose biosensing systems of the present disclosure significantly minimizes variation in the glucose concentrations during a bioprocess such as fermentation. For example, the output of the sensors can be used to control a pump that regulates the rate at which the concentrated glucose solution is added, maintaining the glucose concentration at the desired value.

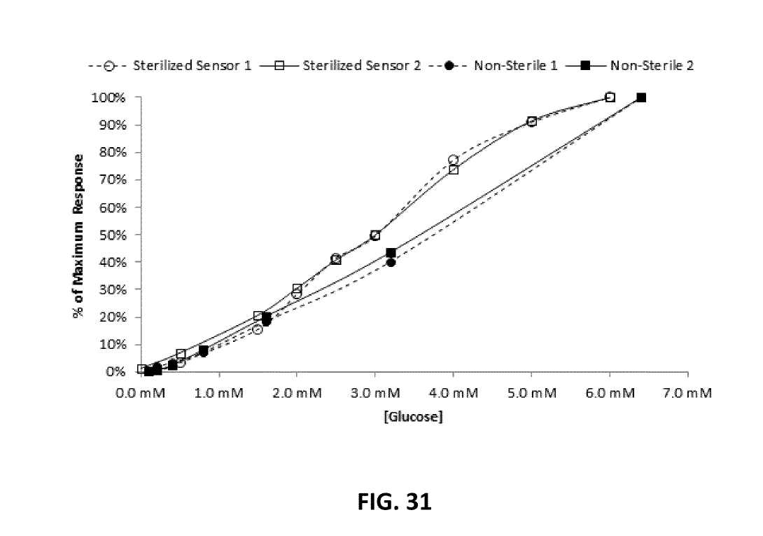

As shown in FIG. 31, response curves were generated to determine the effects of sterilization using gamma irradiation on the activity of glucose biosensing systems. To fabricate the glucose sensors, approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. After curing, sensors were dip coated 5 times in DuPont Nafion 117 with 10 minutes of cure time between dips. Sensors were stored dry after curing. Two sensors received gamma sterilization of 10.4-10.8 kGy for approximately 45 min while two separate sensors were kept as unsterilized controls. Sensors were tested in HEPES buffer at pH 7.4. Concentrations were increased in a stepwise fashion using additions from a 1 M glucose stock solution. Sensor responses at the respective glucose concentrations were recorded and compared to evaluate possible loss of sensor activity due to the gamma sterilization. As shown in FIG. 31, no statistical difference in sensor activity was observed between gamma irradiated and control sensors (compare trend lines between Sterilized Sensors 1 and 2 to Non-Sterilized Sensors 1 and 2), thus demonstrating the feasibility of repeated use (e.g., after sterilization) of the glucose sensors with no loss in sensor function.

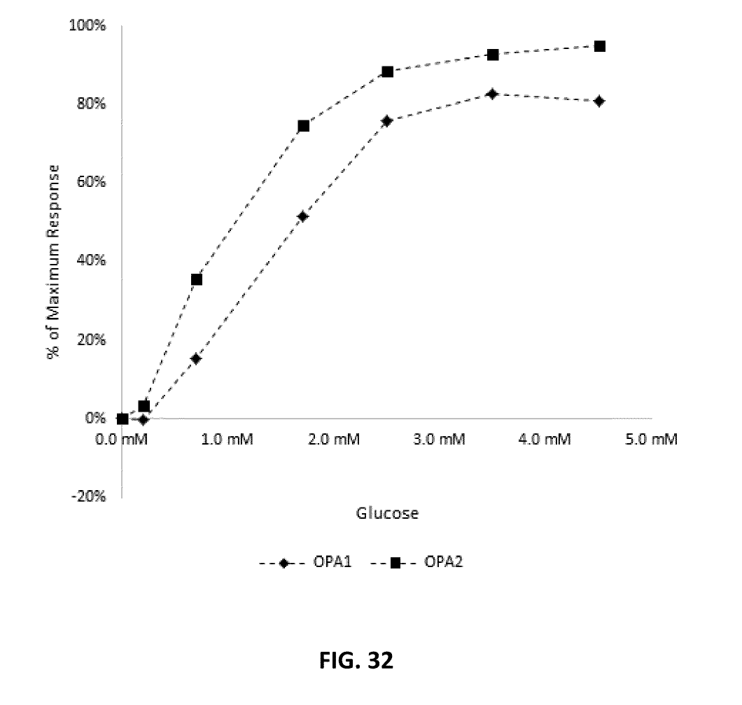

As shown in FIG. 32, response curves were generated to determine the effects of chemical sterilization using CIDEX OPA on the activity of glucose biosensing systems. To fabricate the glucose sensors, approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. All experiments were performed in HEPES buffer at pH 7.4. Glucose concentrations were increased incrementally to the concentrations indicated in FIG. 32 using additions of a 2M stock glucose concentration. Sensors were initially calibrated and then were treated with CIDEX OPA solution according to the manufacturer's instructions (12 minute treatment). As shown in FIG. 32, little difference in sensor activity was observed in two replicate treatments of the sensors with CIDEX OPA, thus demonstrating the feasibility of repeated use (e.g., after sterilization) of the glucose sensors with minimal loss of sensor function (for comparison, see, e.g., FIGS. 16, 18, 19 and 21).

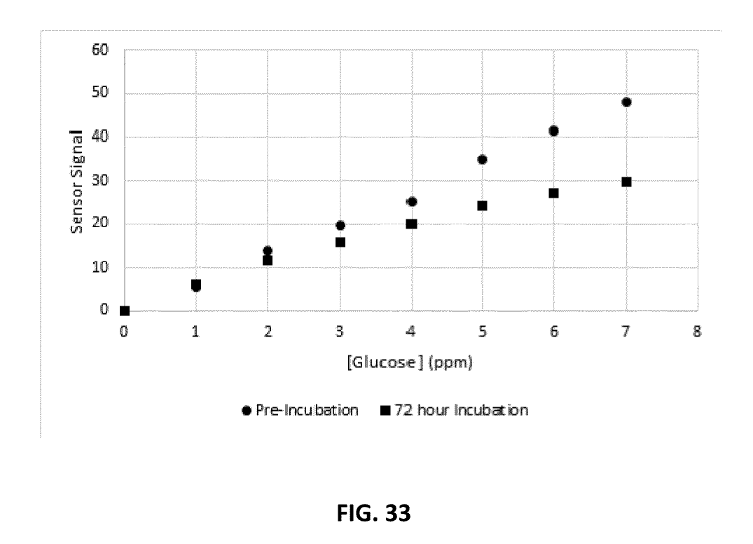

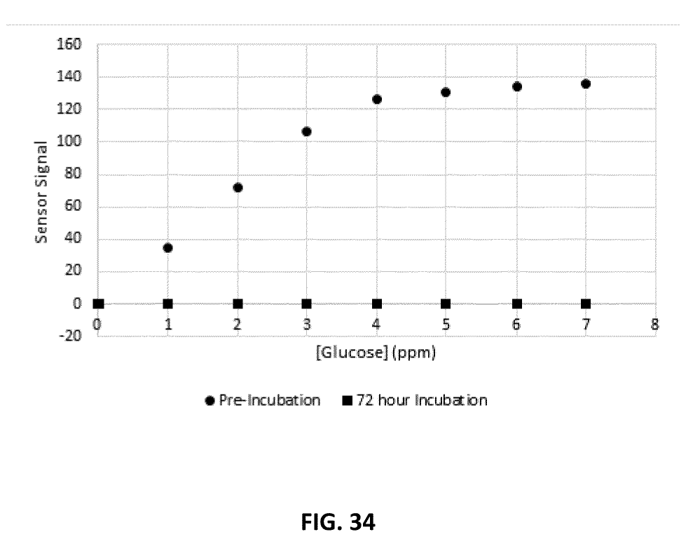

As shown in FIG. 33, response curves were generated to determine the effects of temperature incubation at 50° C. on the activity of glucose biosensing systems having glucose oxidase from Aspergillis niger (Vendor/Cat.#: Biomatik, A4149; Source: Aspergillis niger; Activity: 225 U/mg) immobilized in a cross-linked BSA matrix. To fabricate the glucose sensors, approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. All experiments were performed in HEPES buffer at pH 7.4. Sensors were placed in the beakers and glucose was added to the solution at the concentrations indicated in FIG. 33. The sensors' signals were recorded after reaching a new steady state signal subsequent to addition of glucose. Sensors were first calibrated at room temperature (pre-incubation) and then incubated in the buffer at 50° C. for 72 hours. After the 72-hour incubation period, the sensors were calibrated again and the effects of the elevated temperature incubation on the sensors signals were evaluated. As shown in FIG. 33, glucose biosensors with glucose oxidase from Biomatik retained activity after a significant length of time at an elevated temperature (i.e., high thermostability), demonstrating a difference with the other glucose oxidase variants (see FIGS. 34-36).

As shown in FIG. 34, response curves were generated to determine the effects of temperature incubation at 50° C. on the activity of glucose biosensing systems having glucose oxidase from Aspergillis niger (Vendor/Cat.#: Sigma Aldrich, G2133; Source: Aspergillis niger; Activity: 150 kU/mg) immobilized in a cross-linked BSA matrix. To fabricate the glucose sensors, approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. All experiments were performed in HEPES buffer at pH 7.4. Sensors were placed in the beakers and glucose was added to the solution at the concentrations indicated in FIG. 34. The sensors' signals were recorded after reaching a new steady state signal subsequent to addition of glucose. Sensors were first calibrated at room temperature (pre-incubation) and then incubated in the buffer at 50° C. for 72 hours. After the 72-hour incubation period, the sensors were calibrated again and the effects of the elevated temperature incubation on the sensors' signals were evaluated. As shown in FIG. 34, glucose biosensors with glucose oxidase from Sigma Aldrich did not retain activity at an elevated temperature (i.e., low thermostability), thus demonstrating the unpredictable nature of using purified enzymes such as glucose oxidase in biosensing systems.

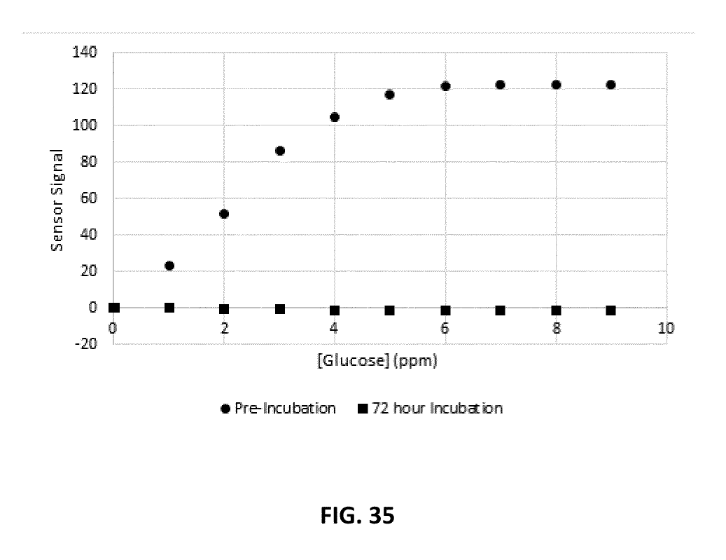

As shown in FIG. 35, response curves were generated to determine the effects of temperature incubation at 50° C. on the activity of glucose biosensing systems having glucose oxidase from Aspergillis niger (Vendor/Cat.#: EMD/Calbiochem, 345386; Source: Aspergillis niger; Activity: 306 U/mg) immobilized in a cross-linked BSA matrix. To fabricate the glucose sensors, approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. All experiments were performed in HEPES buffer at pH 7.4. Sensors were placed in the beakers and glucose was added to the solution at the concentrations indicated in FIG. 35. The sensors' signals were recorded after reaching a new steady state signal subsequent to addition of glucose. Sensors were first calibrated at room temperature (pre-incubation) and then incubated in the buffer at 50° C. for 72 hours. After the 72-hour incubation period, the sensors were calibrated again and the effects of the elevated temperature incubation on the sensors' signals were evaluated. As shown in FIG. 35, glucose biosensors with glucose oxidase from EMD/Calbiochem did not retain activity at an elevated temperature (i.e., low thermostability), thus demonstrating the unpredictable nature of using purified enzymes such as glucose oxidase in biosensing systems.

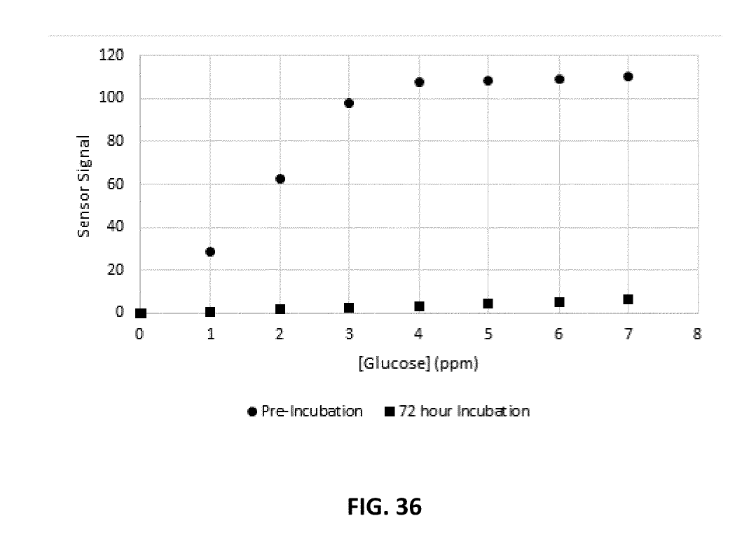

As shown in FIG. 36, response curves were generated to determine the effects of temperature incubation at 50° C. on the activity of glucose biosensing systems having glucose oxidase from Aspergillis niger (Vendor/Cat.#: Sigma Aldrich, G6125; Source: Aspergillis niger; Activity: 250 U/mg) immobilized in a cross-linked BSA matrix. To fabricate the glucose sensors, approximately 10 μL of glucose oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. All experiments were performed in HEPES buffer at pH 7.4. Sensors were placed in the beakers and glucose was added to the solution at the concentrations indicated in FIG. 36. The sensors' signals were recorded after reaching a new steady state signal subsequent to addition of glucose. Sensors were first calibrated at room temperature (pre-incubation) and then incubated in the buffer at 50° C. for 72 hours. After the 72-hour incubation period, the sensors were calibrated again and the effects of the elevated temperature incubation on the sensors' signals were evaluated. As shown in FIG. 36, glucose biosensors with glucose oxidase from Sigma Aldrich did not retain activity at an elevated temperature (i.e., low thermostability), thus demonstrating the unpredictable nature of using purified enzymes such as glucose oxidase in biosensing systems.

### Alcohol Analytes

Embodiments of the present disclosure are also directed to using biosensing systems for making real-time, continuous, and quantitative assessments of alcohols and alcohol-based analytes. In some embodiments, the biosensing systems of the present disclosure can also be designed to detect and monitor various other alcohols and alcohol-based analytes, including but not limited to, ethanol, butanol, methanol and analytes incorporating these and other alcohols (i.e., alcohol-based). In one embodiment, biosensing systems of the present disclosure have a biocomponent, a transducer, a photon-detection device, and a signal-processing system. A signal processing system processes the signal from a photon-detection device into information that can be displayed to an end user. An example of a signal processing system is a microprocessor that accepts an input signal from a photon-detection device that is coupled to a biosensing element. The signal processing system then uses a software program that encodes an algorithm. The algorithm used by the software transforms the data provided by the input signal and provides an output signal that correlates to a numerical display of the concentration of an analyte that the biosensing system detected. Biosensing systems and biosensing elements of the present disclosure are stable and long-lived, can stand prolonged storage and can also perform well in use for extended periods. Biocomponents may be stabilized through various means, depending upon the type of biocomponent and transducer used.

Some enzymes that react with alcohol analytes, such as alcohol oxidases (EC 1.1.3.13), produce hydrogen peroxide as a by-product, as shown in the representative equation below, where alcohol oxidase catalyzes the reaction of a primary alcohol and oxygen to produce an aldehyde and hydrogen peroxide.

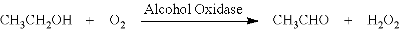

In some embodiments, hydrogen peroxide can then be detected in the biosensing element and used as an indicator of the concentration of alcohol in the aqueous solution. In other embodiments, catalase or peroxidase can be used as a biocomponent and can be coupled to an oxygen optode that measures a change in the concentration of oxygen in the solution. Enzymes like catalase and peroxidase catalyze the decomposition of hydrogen peroxide into water and oxygen. Thus, when hydrogen peroxide is in a solution and interacts with the biosensing element, oxygen is produced. The oxygen produced can interact with the transducer by quenching some of the luminescence of the transducer. Thus, the transducer produces a signal that is correlated to the concentration of oxygen in the sample which is related to the concentration of hydrogen peroxide.

In some embodiments, the active lifetime of sensors made using alcohol oxidase may be limited in some applications. Deactivation of the oxidase may occur when in solution with an alcohol due at least in part to its oxidation by hydrogen peroxide, a by-product of the enzyme-catalyzed oxidation of the alcohol. This limitation can be overcome through addition of a second enzyme, such as a peroxidase (EC 1.11.1), including but not limited to catalase (EC 1.11.1.6) or peroxidase (EC 1.11.1.7), to the sensing layer. These enzymes catalyze the rapid breakdown of hydrogen peroxide, thus lowering the concentration of hydrogen peroxide within the sensing layer and increasing the active lifetime of the alcohol oxidase in the layer. Sensors incorporating both alcohol oxidase and one of these hydrogen peroxide-degrading enzymes can retain activity longer.

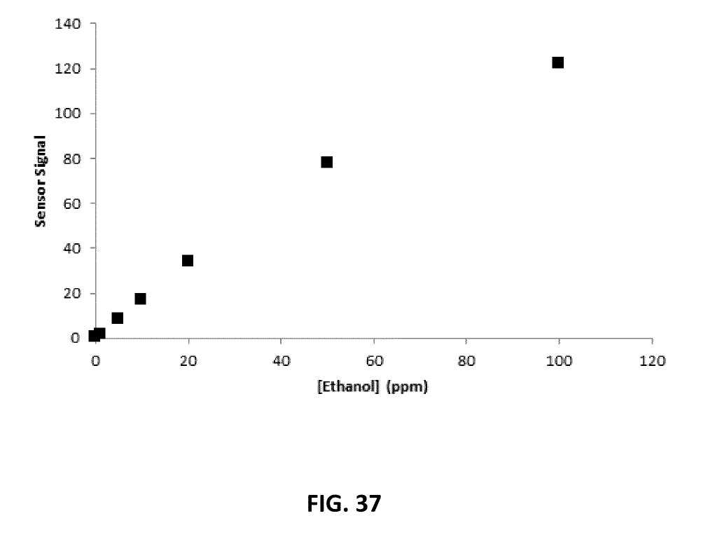

As shown in FIG. 37, a response curve was generated using an ethanol biosensing system that includes an ethanol sensor having alcohol oxidase immobilized in a cross-linked BSA matrix. The sensor was fabricated by crosslinking alcohol oxidase from Pichia pastoris in a bovine serum albumin (BSA) matrix using glutaraldehyde as the crosslinking agent. Approximately 2.5 μL of an alcohol oxidase solution containing 10-40 units alcohol oxidase/mL was mixed with 17.5 μL a 320 mg/mL BSA solution. To this solution, approximately 6.3 μL of 2.5% glutaraldehyde was added to initiate crosslinking 0.5 μL of the resulting solution was immediately pipetted on top of the fluorophore layer and allowed to cure for 20 min. These experiments were performed in stirred 200 mL beakers containing HEPES buffer at pH 7.2. Sensors were placed in the beakers and ethanol was added to the solution at the concentrations indicated in FIG. 37. The sensors' signals were recorded after reaching a new steady state signal subsequent to addition of ethanol. As shown in FIG. 37, the results of these experiments demonstrate the efficacy of using BSA-immobilized alcohol oxidase for measuring ethanol concentrations.

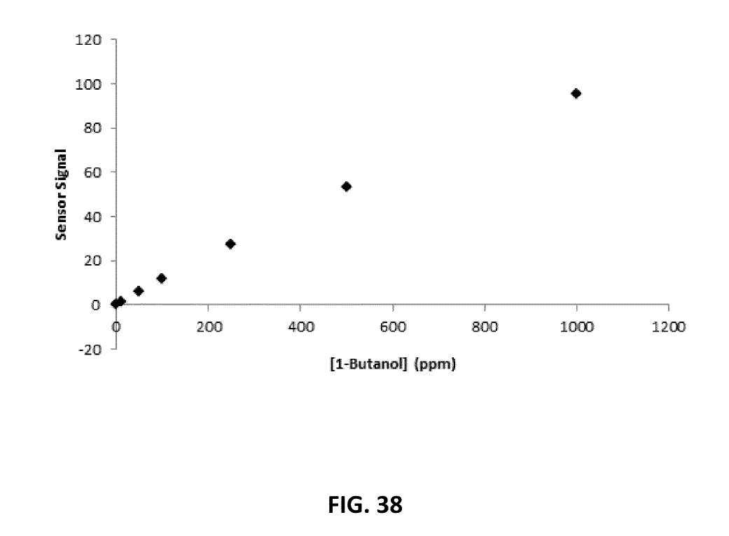

As shown in FIG. 38, a response curve was generated using a butanol biosensing system that includes a butanol sensor having alcohol oxidase immobilized in a cross-linked BSA matrix. The sensor was fabricated by crosslinking alcohol oxidase from Pichia pastoris in a bovine serum albumin (BSA) matrix using glutaraldehyde as the crosslinking agent. Approximately 2.5 μL of an alcohol oxidase solution containing 10-40 units alcohol oxidase/mL was mixed with 17.5 μL a 320 mg/mL BSA solution. To this solution, approximately 6.3 μL of 2.5% glutaraldehyde was added to initiate crosslinking 0.5 μL of the resulting solution was immediately pipetted on top of the fluorophore layer and allowed to cure for 20 min. These experiments were performed in stirred 200 mL beakers containing HEPES buffer at pH 7.2. Sensors were placed in the beakers and butanol was added to the solution at the concentrations indicated in FIG. 38. The sensors' signals were recorded after reaching a new steady state signal subsequent to addition of butanol. As shown in FIG. 38, the results of these experiments demonstrate the efficacy of using BSA-immobilized alcohol oxidase for measuring butanol concentrations.

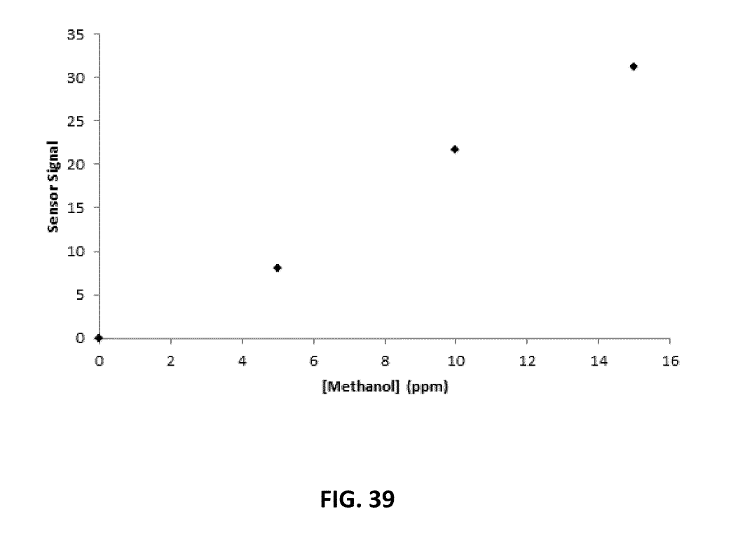

As shown in FIG. 39, a response curve was generated using a methanol biosensing system that includes a methanol sensor having alcohol oxidase immobilized in a cross-linked BSA matrix. The sensor was fabricated by crosslinking alcohol oxidase from Pichia pastoris in a bovine serum albumin (BSA) matrix using glutaraldehyde as the crosslinking agent. Approximately 2.5 μL of an alcohol oxidase solution containing 10-40 units alcohol oxidase/mL was mixed with 17.5 μL a 320 mg/mL BSA solution. To this solution, approximately 6.3 μL of 2.5% glutaraldehyde was added to initiate crosslinking 0.5 μL of the resulting solution was immediately pipetted on top of the fluorophore layer and allowed to cure for 20 min. These experiments were performed in stirred 200 mL beakers containing HEPES buffer at pH 7.2. Sensors were placed in the beakers and methanol was added to the solution at the concentrations indicated in FIG. 39. The sensors' signals were recorded after reaching a new steady state signal subsequent to addition of methanol. As shown in FIG. 39, the results of these experiments demonstrate the efficacy of using BSA-immobilized alcohol oxidase for measuring methanol concentrations.

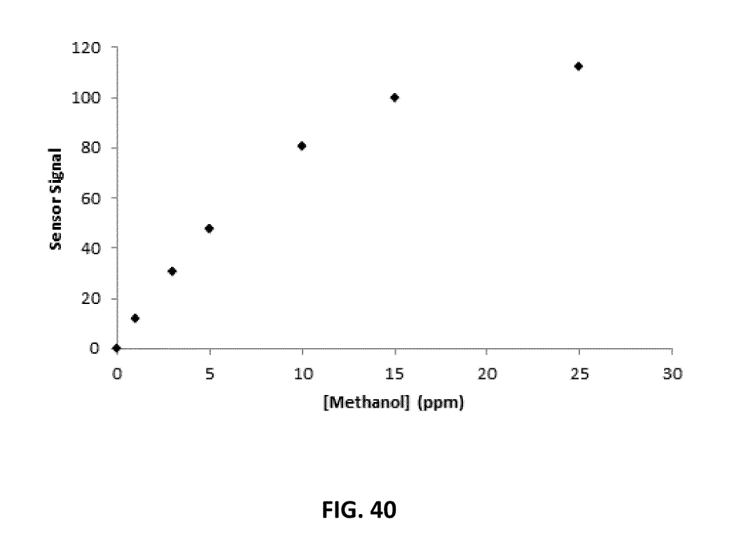

As shown in FIG. 40, a response curve was generated using a methanol biosensing system that includes a methanol sensor having alcohol oxidase immobilized in a sol gel-polyvinyl alcohol polymer matrix. To fabricate the methanol biosensors, approximately 725 μL of tetraethyl ortho-silicate was combined with 350 μL of deionized water and 25 μL of 0.1 M hydrochloric acid in a 2.0 mL microcentrifuge tube. The resulting mixture was shaken for 1 hour at room temperature until it was observed visually to become a single phase. Approximately 25 μL of the resultant mixture was then combined with 100 μL of 1% (w/v) polyvinyl alcohol and vortexed thoroughly. Approximately 10 μL of the vortexed mixture was then combined with 5 μL of an alcohol oxidase solution containing 10-40 units alcohol oxidase/mL and the mixture was vortexed for 10 seconds. Approximately 1 μL of the enzyme containing mixture was then pipetted onto the fluorophore layer of the sensor. The sol-gel was allowed to cure for 1 hour and was then stored in 1 mM HEPES buffer at pH 7.4 at 4° C. until use. These experiments were performed in a stirred 200 mL beaker containing HEPES buffer at pH 7.2. The sensors were placed in the beaker and methanol was added to the solution at the concentrations indicated in FIG. 40. The sensors' signals were recorded after reaching a new steady state signal subsequent to addition of methanol. As shown in FIG. 40, the results of these experiments demonstrate the efficacy of using sol-gel polyvinylalcohol-immobilized alcohol oxidase for measuring methanol concentrations.

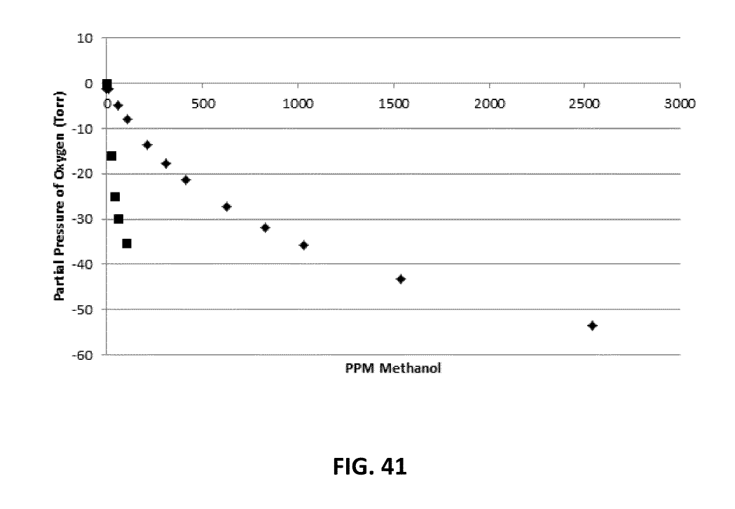

As shown in FIG. 41, response curves were generated using a methanol biosensing system that includes a methanol sensor having a Nafion coating. To fabricate the methanol sensors, approximately 10 μL of alcohol oxidase (12.5 mg/mL) was mixed with 90 μL of BSA (160 mg/mL in DI water), 5 μL of glycerol, and 100 μL of glutaraldehyde (2.5% in DI water). Approximately 1 μL of the resulting solution was pipetted onto the fluorophore layer and allowed to cure for 10 minutes before immersion in HEPES buffer at pH 7.2. After curing, sensors were dip coated 5 times in DuPont Nafion 117 with 10 minutes of cure time between dips. Sensors were stored dry after curing. Sensors were tested in HEPES buffer at pH 7.4.

Addition of the diffusion layer or barrier comprising a Nafion coating to the sensors alters the detection range through creation of a diffusion layer, which slows diffusion of the methanol into the biocomponent layer comprising alcohol oxidase. As the concentration of methanol increased, the local concentration of oxygen within the biosensor element decreased due to the alcohol oxidase catalyzed reaction with the alcohol and oxygen (diamonds; no Nafion coating). As shown in FIG. 41, the addition of the Nafion coating enhanced the sensitivity of detection, such that the linear response range was shifted left (squares; Nafion-coated).

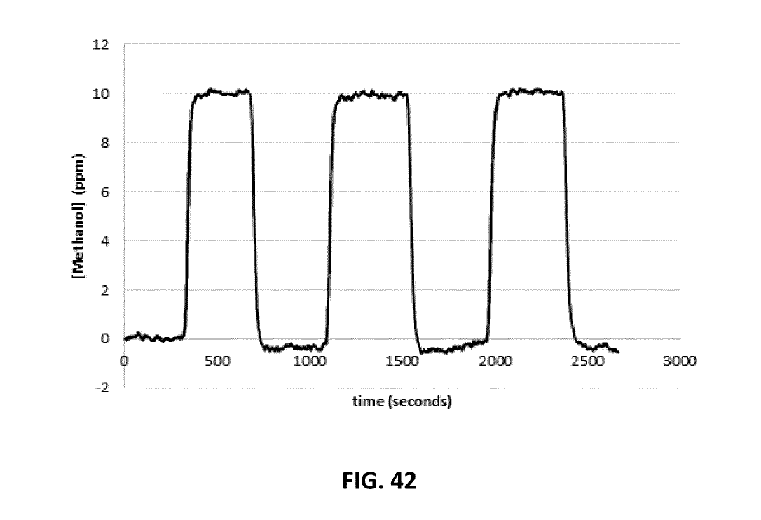

As shown in FIG. 42, sensor signals were generated using a flow-through chamber for continuously sensing methanol concentrations. The sensor was fabricated by crosslinking alcohol oxidase from Pichia pastoris in a bovine serum albumin (BSA) matrix using glutaraldehyde as the crosslinking agent. Approximately 2.5 μL of an alcohol oxidase solution containing 10-40 units alcohol oxidase/mL was mixed with 17.5 μL a 320 mg/mL BSA solution. To this solution, approximately 6.3 μL of 2.5% glutaraldehyde was added to initiate crosslinking 0.5 μL of the resulting solution was immediately pipetted on top of the fluorophore layer and allowed to cure for 20 min. These experiments were performed in stirred 200 mL beakers containing HEPES buffer at pH 7.2. Sensors were placed into a flow stream though a ferruled port. Solutions of water and water containing methanol at a concentration of 10 ppm were alternately allowed to flow across the sensor. Sensor data was collected continuously and is charted in FIG. 42. As shown in FIG. 42, the results of these experiments demonstrate the efficacy of the biosensors of the present disclosure for continuous in situ sensing, including rapid response times and the ability to accurately track changing concentrations of methanol in a flowing stream of fluid.

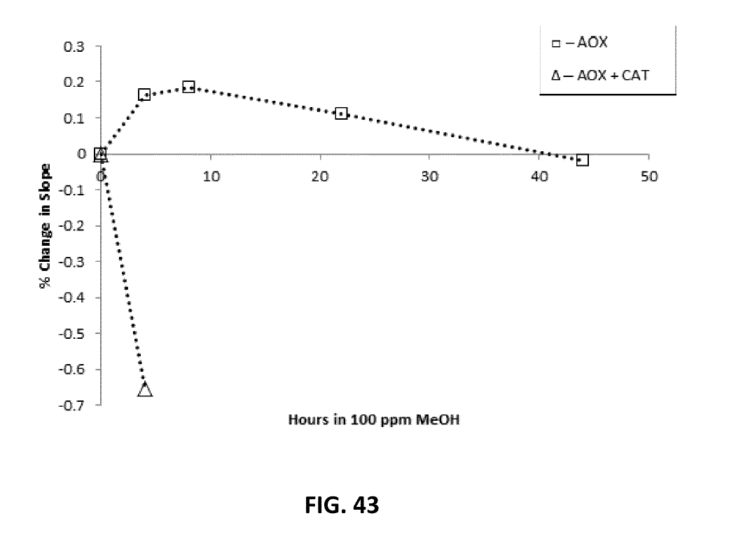

As shown in the graphical representations of FIG. 43, the effects of catalase on the active lifetime of methanol biosensing systems were investigated. H₂O₂ is a byproduct of the oxygenation of alcohols by alcohol oxidase. The H₂O₂ produced acts to inhibit the activity of the immobilized alcohol oxidase and also can oxidize the alcohol oxidase enzyme itself, thus shortening the lifetime of the sensor. Catalase (also referred to as “CAT”) can extend the lifetime of the sensors by rapidly catalyzing the dissociation of H₂O₂ into H₂O and O₂, thus lowering the steady state concentration of H₂O₂ within the enzymatic sensing layer. The sensor containing only alcohol oxidase was fabricated by crosslinking alcohol oxidase from Pichia pastoris in a bovine serum albumin (BSA) matrix using glutaraldehyde as the crosslinking agent. Approximately 2.5 μL of an alcohol oxidase solution containing 10-40 units alcohol oxidase/mL was mixed with 17.5 μL a 320 mg/mL BSA solution. To this solution, approximately 6.3 μL of 2.5% glutaraldehyde was added to initiate crosslinking 0.5 μL of the resulting solution was immediately pipetted on top of the fluorophore layer and allowed to cure for 20 min. The sensors containing both alcohol oxidase and catalase were fabricated by crosslinking alcohol oxidase from Pichia pastoris in a bovine serum albumin (BSA) matrix using glutaraldehyde as the crosslinking agent. Approximately 2.5 μL of an alcohol oxidase solution containing 10-40 units alcohol oxidase/mL was mixed with 17.5 μL a 320 mg/mL BSA solution. To this solution, 6.3 μL of 2.5% glutaraldehyde was added to initiate crosslinking 0.5 μL of the resulting solution was immediately pipetted on top of the fluorophore layer and allowed to cure for 20 min. A second solution was then prepared by mixing 10 μL of CAT, 90 μL BSA (56 mg/mL), and 90 μL BSA (56 mg/mL) by vortexing for 5 seconds. Approximately 50 μL glutaraldehyde was then added to the resulting solution and the mixture was vortexed for an additional 5 seconds. Approximately 0.5 μL of the resulting mixture was then pipetted on top of the alcohol oxidase layer and allowed to cure for 15 minutes.

The sensors were used to create calibration curves of sensor signal vs. concentration of methanol. The sensors were then allowed to incubate in in a solution of water and 100 ppm methanol for extended time periods, followed by recalibration of the sensor. A decrease in the slope of the calibration curve was used as an indicator of sensor activity loss. As shown in FIG. 43, the sensor fabricated with only alcohol oxidase (squares) lost nearly 70% of its activity after the first 4 hours of incubation in 100 ppm methanol. However, the sensor fabricated with both catalase and alcohol oxidase (triangles) in the sensing layer had a calibration curve slope within 2% of the initial slope after 44 hours of incubation in 100 ppm methanol, thus greatly extending the active lifetime of the sensor. As shown in FIG. 43, the addition of catalase extended the lifetime of the sensors when fabricated as a separate layer on top or beneath the alcohol oxidase sensing layer, and as a mixed layer in which alcohol oxidase and catalase were co-immobilized (see, e.g., FIG. 19).

The above examples, embodiments, definitions and explanations should not be taken as limiting the full metes and bounds of the invention. The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

### REFERENCES

The contents of the following references are hereby incorporated into the present disclosure:
Ikeda et al., J. Electroanal. Chem. 361 (1993) 221.
Safina et al., Electrochimica Acta 55 (2010) 7690-7695.
Roda A, Cevenini L, Michelini E, Branchini B R. A portable bioluminescence engineered cell-based biosensor for on-site applications. Biosens Bioelectron. 2011 Apr. 15; 26(8):3647-53. Epub 2011 Feb. 18.
Sezginturk M K, Dinckaya E. A Biosensor for the Determination of β-galactosidase Activity: A Different Viewpoint on Biosensors. Artif Cells Blood Substit Immobil Biotechnol. 2011 Feb. 25.
Sezginturk M K, Dinckaya E. β-galactosidase Determination by an Electrochemical Biosensor Mediated with Ferrocene. Artif Cells Blood Substit Immobil Biotechnol. 2011 Feb. 22.
Leal M P, Assali M, Fernandez I, Khiar N. Copper-catalyzed azide-alkyne cycloaddition in the synthesis of polydiacetylene: “click glycoliposome” as biosensors for the specific detection of lectins. Chemistry. 2011 Feb. 7; 17(6):1828-36.
Veetil J V, Jin S, Ye K. A glucose sensor protein for continuous glucose monitoring. Biosens Bioelectron. 2010 Dec. 15; 26(4):1650-5.
Moreira F T, Kamel A H, Guerreiro J R, Sales M G. Man-tailored biomimetic sensor of molecularly imprinted materials for the potentiometric measurement of oxytetracycline. Biosens Bioelectron. 2010 Oct. 15; 26(2):566-74.
Yang C, Zhang Z, Shi Z, Xue P, Chang P, Yan R. Application of a novel co-enzyme reactor in chemiluminescence flow-through biosensor for determination of lactose. Talanta. 2010 Jun. 30; 82(1):319-24.
Ren X, Yang L, Tang F, Yan C, Ren J. Enzyme biosensor based on NAD-sensitive quantum dots. Biosens Bioelectron. 2010 Sep. 15; 26(1):271-4.
Conzuelo F, Gamella M, Campuzano S, Ruiz M A, Reviejo A J, Pingarron J M. An integrated amperometric biosensor for the determination of lactose in milk and dairy products. J Agric Food Chem. 2010 Jun. 23; 58(12):7141-8.
Nagatsuka T, Uzawa H, Ohsawa I, Seto Y, Nishida Y. Use of lactose against the deadly biological toxin ricin. ACS Appl Mater Interfaces. 2010 April; 2(4):1081-5.
Kawsar S M, Matsumoto R, Fujii Y, Yasumitsu H, Dogasaki C, Hosono M, Nitta K, Hamako J, Matsui T, Kojima N, Ozeki Y. Purification and biochemical characterization of a D-galactose binding lectin from Japanese sea hare (Aplysia kurodai) eggs. Biochemistry (Mosc). 2009 July; 74(7):709-16.
Munoz F J, Perez J, Rumbero A, Santos J I, Canada F J, Andre S, Gabius H J, Jimenez-Barbero J, Sinisterra J V, Hernaiz M J. Glycan tagging to produce bioactive ligands for a surface plasmon resonance study via immobilization on different surfaces. Bioconjug Chem. 2009 April; 20(4):673-82.
Jenkins D M, Teruel M A, Reyes-de-Corcuera J I, Young O. Simultaneous determination of hydrolysis and mutarotation rates during the enzymatic hydrolysis of lactose. J Agric Food Chem. 2008 Sep. 24; 56(18):8303-8.
Sezginturk M K, Dinckaya E. Beta-galactosidase monitoring by a biosensor based on Clark electrode: its optimization, characterization and application. Biosens Bioelectron. 2008 Jul. 15; 23(12):1799-804.
Varshney M, Li Y. Double interdigitated array microelectrode-based impedance biosensor for detection of viable Escherichia coli O157:H7 in growth medium. Talanta. 2008 Jan. 15; 74(4):518-25.
Mora F, Tran D H, Oudry N, Hopfgartner G, Jeannerat D, Sakai N, Matile S. Interface engineering of synthetic pores: towards hypersensitive biosensors. Chemistry. 2008; 14(6): 1947-53.
Seo J H, Adachi K, Lee B K, Kang D G, Kim Y K, Kim K R, Lee H Y, Kawai T, Cha H J. Facile and rapid direct gold surface immobilization with controlled orientation for carbohydrates. Bioconjug Chem. 2007 November-December; 18(6):2197-201.
Sharma S K, Kumar A, Chaudhary R, Suman, Pundir C S, Sehgal N. Lactose biosensor based on lactase and galactose oxidase immobilized in polyvinyl formal. Artif Cells Blood Substit Immobil Biotechnol. 2007; 35(4):421-30.
Betancor L, Luckarift H R, Seo J H, Brand O, Spain J C. Three-dimensional immobilization of beta-galactosidase on a silicon surface. Biotechnol Bioeng. 2008 Feb. 1; 99(2):261-7.
Stoica L, Ruzgas T, Ludwig R, Haltrich D, Gorton L. Direct electron transfer—a favorite electron route for cellobiose dehydrogenase (CDH) from Trametes villosa. Comparison with CDH from Phanerochaete chrysosporium. Langmuir. 2006 Dec. 5; 22(25): 10801-6.
Turishcheva GKh, Kazarinov I A, Ignatov O V, Ignatov V V. [A bioelectrochemical study of a suspension of Escherichia coli cells metabolizing glucose and lactose]. Mikrobiologiia. 2006 January-February; 75(1):52-6. Russian.
Zhang Y, Luo S, Tang Y, Yu L, Hou K Y, Cheng J P, Zeng X, Wang P G. Carbohydrate-protein interactions by “clicked” carbohydrate self-assembled monolayers. Anal Chem. 2006 Mar. 15; 78(6):2001-8.
Stoica L, Ludwig R, Haltrich D, Gorton L. Third-generation biosensor for lactose based on newly discovered cellobiose dehydrogenase. Anal Chem. 2006 Jan. 15; 78(2):393-8.
Maestre E, Katakis I, Narvaez A, Dominguez E. A multianalyte flow electrochemical cell: application to the simultaneous determination of carbohydrates based on bioelectrocatalytic detection. Biosens Bioelectron. 2005 Nov. 15; 21(5):774-81. PubMed PMID: 16242617.
Sharma S K, Singhal R, Malhotra B D, Sehgal N, Kumar A. Lactose biosensor based on Langmuir-Blodgett films of poly(3-hexyl thiophene). Biosens Bioelectron. 2004 Oct. 15; 20(3):651-7.
Jenkins D M, Delwiche M J. Adaptation of a manometric biosensor to measure glucose and lactose. Biosens Bioelectron. 2003 January; 18(1):101-7.
Rajendran V, Lrudayaraj J. Detection of glucose, galactose, and lactose in milk with a microdialysis-coupled flow injection amperometric sensor. J Dairy Sci. 2002 June; 85(6):1357-61.
Curey T E, Salazar M A, Oliveira P, Javier J, Dennis P J, Rao P, Shear J B. Enzyme-based sensor arrays for rapid characterization of complex disaccharide solutions. Anal Biochem. 2002 Apr. 1; 303(1):42-8.
Lehmann M, Riedel K, Adler K, Kunze G. Amperometric measurement of copper ions with a deputy substrate using a novel Saccharomyces cerevisiae sensor. Biosens Bioelectron. 2000 June; 15(3-4):211-9.
Ramakrishnan A, Sadana A. Analyte-receptor binding and dissociation kinetics for biosensor applications: a fractal analysis. Biosens Bioelectron. 2000; 15(11-12):651-62.
Eshkenazi I, Sacks V, Neufeld T, Rishpon J. Amperometric biosensors based on microflow injection system. Appl Biochem Biotechnol. 2000 November-December; 89(2-3):217-30.
Eshkenazi I, Maltz E, Zion B, Rishpon J. A three-cascaded-enzymes biosensor to determine lactose concentration in raw milk. J Dairy Sci. 2000 September; 83(9):1939-45.
Tkac J, Sturdik E, Gemeiner P. Novel glucose non-interference biosensor for lactose detection based on galactose oxidase-peroxidase with and without co-immobilised beta-galactosidase. Analyst. 2000 July; 125(7):1285-9.
Svitel J, Curilla O, Tkac J. Microbial cell-based biosensor for sensing glucose, sucrose or lactose. Biotechnol Appl Biochem. 1998 April; 27 (Pt 2):153-8.
Sorochinskii V V, Kurganov B I. [Biosensors for detecting organic compounds. II. Sensors for carbohydrates, aromatic, heterocyclic and other organic compounds]. Prikl Biokhim Mikrobiol. 1998 January-February; 34(1):22-42. Review. Russian.
Ruzgas T, Csoregi E, Katakis I, Kenausis G, Gorton L. Preliminary investigations of an amperometric oligosaccharide dehydrogenase-based electrode for the detection of glucose and some other low molecular weight saccharides. J Mol Recognit. 1996 September-December; 9(5-6):480-4.
Szabo E E, Adanyi N, Varadi M. Application of biosensor for monitoring galactose content. Biosens Bioelectron. 1996; 11(10):1051-8.
Filipiak M, Fludra K, Go cimi ska E. Enzymatic membranes for determination of some disaccharides by means of an oxygen electrode. Biosens Bioelectron. 1996; 11(4):355-64.
Sriyudthsak M, Cholapranee T, Sawadsaringkarn M, Yupongchaey N, Jaiwang P. Enzyme-epoxy membrane based glucose analyzing system and medical applications. Biosens Bioelectron. 1996; 11(8):735-42.
Katsu T, Zhang X, Rechnitz G A. Simultaneous determination of lactose and glucose in milk using two working enzyme electrodes. Talanta. 1994 June; 41(6):843-8.
Carlsson H, Ljungcrantz P, Lindbladh C, Persson M, Billow L. Use of genetically prepared enzyme conjugates in lactose and galactose analyses. Anal Biochem. 1994 May 1; 218(2):278-83.
Kiefer H, Klee B, John E, Stierhof Y D, Jahnig F. Biosensors based on membrane transport proteins. Biosens Bioelectron. 1991; 6(3):233-7.
Svorc J, Miertus S, Barlikova A. Hybrid biosensor for the determination of lactose. Anal Chem. 1990 Aug. 1; 62(15):1628-31.
Pfeiffer D, Ralis E V, Makower A, Scheller F W. Amperometric bi-enzyme based biosensor for the detection of lactose-characterization and application. J Chem Technol Biotechnol. 1990; 49(3):255-65.
Heppel, L. A. and Porterfield, V. T. Enzymatic dehalogenation of certain brominated and chlorinated compounds. J. Biol. Chem. 176 (1948) 763-769.
Goldman, P., Milne, G. W. A. and Keister, D. B. Carbon-halogen bond cleavage. 3. Studies on bacterial halidohydrolases. J. Biol. Chem. 243 (1968) 428-434. [Medline UI: 68123008]
Motosugi, M., Esaki, N. and Soda, K. Preparation and properties of 2-halo acid dehalogenase from Pseudomonas putida. Agric. Biol. Chem. 46 (1982) 837-838.
Goldman, P. The enzymatic cleavage of the carbon-fluorine bond in fluoroacetate. J. Biol. Chem. 240 (1965) 3434-3438.
Goldman, P. and Milne, G. W. A. Carbon-fluorine bond cleavage. II. Studies on the mechanism of the defluorination of fluoroacetate. J. Biol. Chem. 241 (1966) 5557-5559. [Medline UI: 67053221]
Chopra, I. J. and Teco, G. N. C. Characteristics of inner ring (3 or 5) monodeiodination of 3,5-diiodothyronine in rat liver: evidence suggesting marked similarities of inner and outer ring deiodinases for iodothyronines. Endocrinology 110 (1982) 89-97. [Medline UI: 82095045]
Goswani, A., Leonard, J. L. and Rosenberg, I. N. Inhibition by coumadin anticoagulants of enzymatic outer ring monodeiodination of iodothyronines. Biochem. Biophys. Res. Commun. 104 (1982) 1231-1238. [Medline UI: 82182305]
Smallridge, R. C., Burman, K. D., Ward, K. E., Wartofsky, L., Dimond, R. C., Wright, F. D. and Lathan, K. R. 3′,5′-Diiodothyronine to 3′-monoiodothyronine conversion in the fed and fasted rat: enzyme characteristics and evidence for two distinct 5′-deiodinases. Endocrinology 108 (1981) 2336-2345. [Medline UI: 81188610]
Keuning, S., Janssen, D. B. and Witholt, B. Purification and characterization of hydrolytic haloalkane dehalogenase from Xanthobacter autotrophicus GJ10. J. Bacteriol. 163 (1985) 635-639. [Medline UI: 85261076]
Scholtz, R., Leisinger, T., Suter, F. and Cook, A. M. Characterization of 1-chlorohexane halidohydrolase, a dehalogenase of wide substrate range from an Arthrobacter sp. J. Bacteriol. 169 (1987) 5016-5021. [Medline UI: 88032819]
Yokota, T., Omori, T. and Kodama, T. Purification and properties of haloalkane dehalogenase from Corynebacterium sp. strain m15-3. J. Bacteriol. 169 (1987) 4049-4054. [Medline UI: 87307981]
Muller, R., Thiele, J., Klages, U. and Lingens, F. Incorporation of [18O]water into 4-hydroxybenzoic acid in the reaction of 4-chlorobenzoate dehalogenase from Pseudomonas sp. CBS 3. Biochem. Biophys. Res. Commun. 124 (1984) 178-182. [Medline UI: 85046491]
Chang, K. H., Liang, P. H., Beck, W., Scholten, J. D., Dunaway-Mariano, D. Isolation and characterization of the three polypeptide components of 4-chlorobenzoate dehalogenase from Pseudomonas sp. strain CBS-3. Biochemistry 31 (1992) 5605-5610. [Medline UI: 92304935]
Crooks, G. P., Copley, S. D. Purification and characterization of 4-chlorobenzoyl CoA dehalogenase from Arthrobacter sp. strain 4-CB1. Biochemistry, 33 (1994) 11645-11649. [Medline UI: 95001870]
de Souza, M. L., Wackett, L. P., Boundy-Mills, K. L., Mandelbaum, R. T. and Sadowsky, M. J. Cloning, characterization, and expression of a gene region from Pseudomonas sp. strain ADP involved in the dechlorination of atrazine. Appl. Environ. Microbiol. 61 (1995) 3373-3378. [Medline UI: 96035669]
de Souza, M. L., Sadowsky, M. J. and Wackett, L. P. Atrazine chlorohydrolase from Pseudomonas sp. strain ADP: gene sequence, enzyme purification, and protein characterization. J. Bacteriol. 178 (1996) 4894-4900. [Medline UI: 96326334]
Lipke, H. and Kearns, C. W. DDT dechlorinase. I. Isolation, chemical properties, and spectrophotometric assay. J. Biol. Chem. 234 (1959) 2123-2128.
Lipke, H. and Kearns, C. W. DDT dechlorinase. II. Substrate and cofactor specificity. J. Biol. Chem. 234 (1959) 2129-2132.
Moorefield, H. H. Purification of DDT-dehydrochlorinase from resistant houseflies. Contr. Boyce Thompson Inst. 18 (1956) 303-310.
Nagasawa, T., Ishii, T. and Yamada, H. Physiological comparison of D-cysteine desulfhydrase of Escherichia coli with 3-chloro-D-alanine dehydrochlorinase of Pseudomonas putida CR 1-1. Arch. Microbiol. 149 (1988) 413-416. [Medline UI: 88251237]
Yamada, H., Nagasawa, T., Ohkishi, H., Kawakami, B. and Tani, Y. Synthesis of D-cysteine from 3-chloro-D-alanine and hydrogen sulfide by 3-chloro-D-alanine hydrogen chloride-lyase (deaminating) of Pseudomonas putida. Biochem. Biophys. Res. Commun. 100 (1981) 1104-1110. [Medline UI: 81281807]
Kohler-Staub, D. and Leisinger, T. Dichloromethane dehalogenase of Hyphomicrobium sp. strain DM2. J. Bacteriol. 162 (1985) 676-681. [Medline UI: 85182487]
Moriguchi, M., Hoshino, S. and Hatanaka, S.-I. Dehalogenation and deamination of 1-2-amino-4-chloro-4-pentenoic acid by Proteus mirabilis. Agric. Biol. Chem. 51 (1987) 3295.
Kumagai, H., Suzuki, H., Shigematsu, H. and Tuchikura, T. S-Carboxymethylcysteine synthase from Escherichia coli. Agric. Biol. Chem. 53 (1989) 2481-2487.
Hayaishi, O. Direct oxygenation by O₂, oxygenases. In: Boyer, P. D., Lardy, H. and Myrback, K. (Eds.), The Enzymes, 2nd ed., vol. 8, Academic Press, New York, 1963, p. 353-371.
Junker, F., Field, J. A., Bangerter, F., Ramsteiner, K., Kohler, H.-P., Joannou, C. L., Mason, J. R., Leisinger, T. and Cook, A. M. Dioxygenation and spontaneous deamination of 2-aminobenzene sulphonic acid in Alcaligenes sp. strain 0-1 with subsequent meta ring cleavage and spontaneous desulphonation to 2-hydroxymuconic acid. Biochem. J. 300 (1994) 429-436.
Fujisawa, H. and Hayaishi, 0. Protocatechuate 3,4-dioxygenase. I. Crystallization and characterization. J. Biol. Chem. 243 (1968) 2673-2681.
Adachi, K., Iwayama, Y., Tanioka, H. and Takeda, Y. Purification and properties of homogentisate oxygenase from Pseudomonas fluorescens. Biochim. Biophys. Acta 118 (1966) 88-97.
Hayaishi, O. and Sutton, W. B. Enzymatic oxygen fixation into acetate concomitant with the enzymatic decarboxylation of L-lactate. J. Am. Chem. Soc. 79 (1957) 4809-4810.
Renganathan, V. Possible involvement of toluene-2,3-dioxygenase in defluorination of 3-fluoro-substituted benzenes by toluene-degrading Pseudomonas sp. strain T-12. Appl. Exp. Microbiol. 55 (1989) 330-334.
Ensley, B. D. and Gibson, D. T. Naphthalene dioxygenase: purification and properties of a terminal oxygenase component. J. Bacteriol. 155 (1983) 505-511.
Fetzner, S., Mueller, R. and Lingens, F. Degradation of 2-chlorobenzoate by Pseudomonas cepacia 2CBS. Biol. Chem. Hoppe-Seyler 370 (1989) 1173-1182.
Suzuki, K., Takemori, S. and Katagiri, M. Mechanism of the salicylate hydroxylase reaction. IV. Fluorimetric analysis of the complex formation. Biochim. Biophys. Acta 191 (1969) 77-85.
Hosokawa, K. and Stanier, R. Y. Crystallization and properties of p-hydroxybenzoate hydroxylase from Pseudomonas putida. J. Biol. Chem. 241 (1966) 2453-2460.
Nakagawa, H. and Takeda, Y. Phenol hydroxylase. Biochim. Biophys. Acta 62 (1962) 423-426.
Ziegler, D. M. and Pettit, F. H. Microsomal oxidases. I. The isolation and dialkylarylamine oxygenase activity of pork liver microsomes. Biochemistry 5 (1966) 2932-2938.
Colby, J. Stirling, D. I. and Dalton, H. The soluble methane mono-oxygenase of Methylococcus capsulatus (Bath). Its ability to oxygenate n-alkanes, n-alkenes, ethers, and alicyclic, aromatic and heterocyclic compounds. Biochem. J. 165 (1977) 395-402.
Schenk, T., Muller, R., Morsberger, F., Otto, M. K. and Lingens, F. Enzymatic dehalogenation of pentachlorophenol by extracts from Arthrobacter sp. strain ATCC 33790. J. Bacteriol. 171 (1989) 5487-5491.
Cardini, G. and Jurtshuk, P. The enzymatic hydroxylation of n-octane by Corynebacterium sp. strain 7E1C. J. Biol. Chem. 245 (1970) 2789-2796.
Augusteyn, R. C., de Jersey, J., Webb, E. C. and Zerner, B. On the homology of the active-site peptides of liver carboxylesterases. Biochim. Biophys. Acta 171 (1969) 128-137.
Dodgson, K. S., Spencer, B. and Williams, K. Studies on sulphatases. 13. The hydrolysis of substituted phenyl sulphates by the arylsulphatase of Alcaligenes metacaligenes. Biochem. J. 64 (1956) 216-221.
Aldridge, W. N. Serum esterases. I. Two types of esterase (A and B) hydrolysing p-nitrophenyl acetate, propionate and butyrate and a method for their determination. Biochem. J. 53 (1953) 110-117.
Augustinsson, K.-B. and Heimburger, G. Enzymatic hydrolysis of organophosphorus compounds. I. Occurrence of enzymes hydrolysing dimethyl-amido-ethoxy-phosphoryl cyanide (Tabun). Acta Chem. Scand. 8 (1954) 753-761.
Cardy, D. L. N., V. Laidler, G. P. C. Salmond, and J. C. Murrell, “Molecular Analysis of the Methane Monooxygenase (MMO) Gene Cluster of Methylosinus trichosporium OB3b,” Molecular Microbiology, 1991. 5(2): pp. 335-342.
Stainthorpe, A. C., V. Lees, G. P. C. Salmond, H. Dalton, and J. C. Murrell, “The Methane Monooxygenase Gene Cluster of Methylococcus capsulatus (Bath),” Gene, 1990. 91: pp. 27-34.
Rosenzwieg, A. C., P. Nordlund, P. M. Takahara, C. A. Frederick, and S. J. Lippard, “Geometry of the Soluble Methane Monoxygenase Catalytic Diiron Center in Two Oxidation States,” Chemistry and Biology, 1995. 2(6): pp. 409-418.
Shields, M. S. and S. C. Francesconi, Microbial Degradation of Trichloroethylene, Dichloroethylene, and Aromatic Pollutants, in U.S. Pat. No. 5,543,317. 1996.
Bertoni, G., F. Bolognese, E. Galli, and P. Barbieri, “Cloning of the Genes for and Characterization of the Early Stages of Toluene and o-Xylene Catabolism in Pseudomonas stutzeri OX1,” Applied and Environmental Microbiology, 1996, 62(10): pp. 3704-3711
Bertoni, G., M. Martino, E. Galli, and P. Barbieri, “Analysis of the Gene Cluster Encoding Toluene/o-Xylene Monoxygenase from Pseudomonas stutzeri OX1,” Applied and Environmental Microbiology, 1998. 64(10): pp. 3626-3632.
Pikus, J. D., J. M. Studts, C. Achim, K. E. Kauffinann, E. Munck, R. J. Steffan, K. McClay, and B. G. Fox, “Recombinant Toluene-4-Monoxygenase: Catalytic and Mossbauer Studies of the Purified Diiron and Rieski Components of a Four-Protein Complex,” Biochemistry, 1996. 35: pp. 9106-9119; Yen, K.-M., “Construction of Cloning Cartridges for Development of Expression Vectors in Gram-Negative Bacteria,” J. Bacteria., 1991. 173(17): pp. 5328-5335.
Newman, 1995; McClay, K., B. G. Fox, and R. J. Steffan, “Chloroform Mineralization by Toluene-Oxidizing Bacteria,” Applied and Environmental Microbiology, 1996. 62(8): pp. 2716-2722.
Byrne, A. M., J. J. Kukor, and R. H. Olsen, “Sequence Analysis of the Gene Cluster Encoding Toluene-3-monooxygenase from Pseudomonas pickettii PK01,” Gene, 1995. 154: pp. 65-70.
Nordlund, I., J. Powlowski, and V. Shingler, “Complete nucleotide sequence and polypeptide analysis of multicomponent phenol hydroxylase from Pseudomonas sp. strain CF600,” Journal of Bacteriology, 1990. 172: pp. 6826-6833.
Stoica, L. and Ludwig, R. and Haltrich, D. and Gorton, L., Third-generation biosensor for lactose based on newly discovered cellobiose dehydrogenase, Analytical chemistry, vol. 78, no. 2, pp. 393-398, 2006.
Sharma, S. K. and Singhal, R. and Malhotra, B D and Sehgal, N. and Kumar, A., Lactose biosensor based on Langmuir-Blodgett films of poly 3-hexyl thiophene, Biosensors and Bioelectronics, vol. 20, no. 3, pp. 651-657, 2004.
Ferreira, L S and Trierweiler, J O and De Souza Jr, M B and Folly, R O M, A lactose fia-biosensor system for monitoring and process control, Brazilian Journal of Chemical Engineering, vol. 21, no. 2, pp. 307-315, 2004.
Marrakchi, M. and Dzyadevych, S. V. and Lagarde, F. and Martelet, C. and Jaffrezic-Renault, N., Conductometric biosensor based on glucose oxidase and beta-galactosidase for specific lactose determination in milk, Materials Science and Engineering: C, vol. 28, nos. 5-6, pp. 872-875, 2008.
Eshkenazi, I. and Maltz, E. and Zion, B. and Rishpon, J., A Three-Cascaded-Enzymes Biosensor to Determine Lactose Concentration in Raw Milk, Journal of dairy science, vol. 83, no. 9, pp. 1939-1945, 2000.
Liu, H. and Li, H. and Ying, T. and Sun, K. and Qin, Y. and Qi, D., Amperometric biosensor sensitive to glucose and lactose based on co-immobilization of ferrocene, glucose oxidase, beta-galactosidase and mutarotase in beta-cyclodextrin polymer, Analytica Chimica Acta, vol. 358, no. 2, pp. 137-144, 1998.
Tkac, J. and Sturdik, E. and Gemeiner, P., Novel glucose non-interference biosensor for lactose detection based on galactose oxidase-peroxidase with and without co-immobilised [small beta]-galactosidase, Analyst, vol. 125, no. 7, pp. 1285-1289, 2000.
Svorc, J. and Miertus, S. and Barlikova, A., Hybrid biosensor for the determination of lactose, Analytical chemistry, vol. 62, no. 15, pp. 1628-1631, 1990.
Pfeiffer, D. and Ralis, E. V. and Makower, A. and Scheller, F. W., Amperometric Bi-enzyme based biosensor for the detection of lactose—characterization and application, Journal of Chemical Technology and Biotechnology, vol. 49, no. 3, pp. 255-265, 1990.
Pilloton, R. and Mascini, M., Flow analysis of lactose and glucose in milk with an improved electrochemical biosensor, Food chemistry, vol. 36, no. 3, pp. 213-222, 1990.
Jenkins, D. M. and Delwiche, M. J., Adaptation of a manometric biosensor to measure glucose and lactose, Biosensors and Bioelectronics, vol. 18, no. 1, pp. 101-107, 2003.
Frreira, L S and Souza Jr, M B and Trierweiler, J O and Hitzmann, B. and Folly, R O M, Analysis of experimental biosensor/FIA lactose measurements, Brazilian Journal of Chemical Engineering, vol. 20, no. 1, pp. 7-13, 2003.
Ottenbacher, D. and Jahnig, F. and Gopel, W., A prototype biosensor based on transport proteins: Electrical transducers applied to lactose permease, Sensors and Actuators B: Chemical, vol. 13, nos. 1-3, pp. 173-175, 1993.
Loechel, C. and Chemnitius, G. C. and Borchardt, M. and Cammann, K., Amperometric bi-enzyme based biosensor for the determination of lactose with an extended linear range, Zeitschrift Lebensmitteluntersuchung und-Forschung A., vol. 207, no. 5, pp. 381-385, 1998.
Goktug, T. and Sezginturk, M. K. and Dinckaya, E., Glucose oxidase-[beta]-galactosidase hybrid biosensor based on glassy carbon electrode modified with mercury for lactose determination, Analytica chimica acta, vol. 551, nos. 1-2, pp. 51-56, 2005.
Yang, W. and Pang, P. and Gao, X. and Cai, Q. and Zeng, K. and Grimes, C. A., Detection of lactose in milk samples using a wireless multi-enzyme biosensor, Sensor Letters, vol. 5, no. 2, pp. 405-410, 2007.
Jturdk, E. and Gemeiner, P., Novel glucose non-interference biosensor for lactose detection based on galactose oxidase-peroxidase with and without co-immobilised beta-galactosidase, Analyst, vol. 125, no. 7, pp. 1285-1289, 2000
Louren, R J M and Serralheiro, M L M and Rebelo, M J F, Development of a new amperometric biosensor for lactose determination, Portugaliae Electrochimica Acta, vol. 21, no. 2, pp. 171-177, 2003.
Pyeshkova, V M and Saiapina, O Y and Soldatkin, O O and Kukla, O L and Dzyadevych, S V, Enzyme conductometric biosensor for determination of lactose, Biotechnology, pp. 76-84, 2008
Fritzen, M. and Schuhmann, W. and Lengeier, J W and Schmidt, H. L., Immobilized transport mutants of bacterial cells in biosensor arrays. Improved selectivity for the simultaneous determination of glucose and lactose, Progress in Biotechnology, vol. 11, pp. 821-827, 1996.
Svitel, J. and Curilla, O. and Tkac, J., Microbial cell-based biosensor for sensing glucose, sucrose or lactose, Biotechnology and applied biochemistry, vol. 27, no. 2, pp. 153-158, 1998.
Lu, E. and Sungur, S, and Yildiz, Y., Development of lactose biosensor based on beta-galactosidase and glucose oxidase immobilized into gelatin, Journal of Macromolecular Science, Part A, vol. 43, no. 3, pp. 525-533, 2006.
Lu, E. and Sungur, S, and Yildiz, Y., Development of Lactose Biosensor Based on Galactosidase and Glucose Oxidase Immobilized into Gelatin, Journal of Macromolecular Science-Part A: Pure and Applied Chemistry, vol. 43, no. 3, pp. 525-534, 2006.
Tkac, J. and Sturdik, E. and Gemeiner, P., Full Papers-Sensors-Novel glucose non-interference biosensor for lactose detection based on galactose oxidase-peroxidase with and without co-immobilised b-galactosidase, Analyst-Letchworth, 125, no. 7, 1285-1290, 2000.
Yang, C. and Zhang, Z. and Shi, Z. and Xue, P. and Chang, P. and Yan, R., Application of a novel co-enzyme reactor in chemiluminescence flow-through biosensor for determination of lactose, Talanta, 82, no. 1, 319-324, 2010.
Tkac, J. and Svitel, J., Bulletin Potravinarskeho Vyskumu (Slovak Republic); Determination of glucose and lactose in milk by a microbial biosensor; Stanovenie glukozy a laktozy v mlieku mikrobialnym sensorom, Bulletin of Food Research, 1997.
Park, I S and Kim, J H and Noh, B S and Kim, T J, Simultaneous determination of lactose and lactic acid in yoghurt by biosensor using dual cathode electrode, Korean Journal of Biotechnology and Bioengineering, Korea Republic, 1997.
C. Muller, F. Schubert and T. Scheper, Multicomponent fiberoptical biosensor for use in hemodialysis monitoring, SPIE Proc., Vol. 2131, pp. 555-562, Biomedical Fiber Optic Instrumentation, Los Angeles, Calif., USA, 1994.
Liu, H. and Ying, T. and Sun, K. and Li, H. and Qi, D., Reagentless amperometric biosensors highly sensitive to hydrogen peroxide, glucose and lactose based on N-methyl phenazine methosulfate incorporated in a Nafion film as an electron transfer mediator between horseradish peroxidase and an electrode, Analytica chimica acta, vol. 344, no. 3, pp. 187-199, 1997.

What is claimed is:

1. A biosensing system for measuring the concentration of an analyte in a solution, the biosensing system comprising:
an optode comprising an optical fiber having a distal tip and a proximal tip;
a photon-detection device coupled to the proximal tip; and
a signal processing system coupled to the photon-detection device;
wherein the distal tip comprises a transducer layer and a biocomponent layer, the biocomponent layer comprising at least one oxidase from Enzyme Commission number 1 (EC 1) that catalyzes a chemical reaction with the analyte;
wherein the transducer layer converts an input signal generated from the chemical reaction with the analyte in the biocomponent layer into an output signal detectable by the photon-detection device; and
wherein the signal processing system generates a value from the output signal detectable by the photon-detection device that corresponds to the concentration of the analyte in the solution.

2. The biosensing system of claim 1, wherein the transducer layer comprises one or more of a fluorescent luminescent agent, a phosphorescent luminescent agent, a bioluminescent luminescent agent, a chemiluminescent luminescent agent, and derivatives and combinations thereof.

3. The biosensing system of claim 1, wherein the transducer layer comprises one or more of trisodium 8-hydroxy-1,3,6-trisulphonate, fluoro (8-anilino-1-naphthalene sulphonate), tris(bipyridine)ruthenium(II) complex, RuDPP, ruthenium complexes, platinum tetrakis(pentafuorophenyl)porphyrin, platinum complexes, acridinium-based reagents, quinidinium-based reagents, fluorescein, fluoresceinamine, a fluorescein containing compound, and derivatives and combinations thereof.

4. The biosensing system of claim 1, wherein the at least one oxidase comprises one or more enzymes from EC numbers 1.1.3, 1.2.3, 1.3.3, 1.4.3, 1.5.3, 1.6.3, 1.7.3, 1.8.3, 1.9.3, 1.10.3, 1.16.3, 1.17.3, 1.21.3, 3.2.1.23, 1.1.3.2, 1.1.3.4, 1.1.3.10, 1.1.3.13, and derivatives and combinations thereof.

5. The biosensing system of claim 1, wherein the biocomponent layer comprises a hydrogel matrix comprising one or more of algal polysaccharides, agarose, alginate, gelatin, collagen, pectin, poly(carbamoyl)sulfonate, locust bean gum, gellan, and combinations and derivatives thereof.

6. The biosensing system of claim 1, wherein said biocomponent layer comprises a matrix comprising a cross-linking agent and one or more of a bovine serum albumin, a lysozyme, alginate, a sol-gel polyvinyl alcohol, and combinations and derivatives thereof.

7. The biosensing system of claim 6, wherein the cross-linking agent is one or more of glutaraldehyde, hexamethylene diisocyanate and 1,5-dinitro-2,4-difluorobenzene, polyethyleneimine, hexamethylenediamine formaldehyde, and combinations and derivatives thereof.

8. The biosensing system of claim 1, wherein the biocomponent layer comprises a matrix that is neither hydrogel-based nor a cross-linked polymer.

9. The biosensing system of claim 1, wherein the biocomponent layer comprises a sol-gel-based matrix.

10. The biosensing system of claim 1, wherein the distal tip further comprises one or more polymer-based diffusion layers.

11. The biosensing system of claim 10, wherein the one or more polymer-based diffusion layers comprise one or more of a polyurethane-based polymer and a tetrafluoroethylene-based fluoropolymer, and combinations and derivatives thereof.

12. The biosensing system of claim 1, wherein the biocomponent layer further comprises one or more enzymes from EC numbers 1.11.1, 1.11.1.6, 1.11.1.7, and combinations and derivatives thereof.

13. The biosensing system of claim 1, wherein the biocomponent layer further comprises one or more stabilizing agents.

14. The biosensing system of claim 13, wherein the one or more stabilizing agents comprises one or more of β-mercaptoethanol, cysteine, dithitreitol (DTT) α-thioglycerol, and other thiol containing reducing agents and combinations and derivatives thereof.

15. The biosensing system of claim 1, wherein the analyte is a carbohydrate.

16. The biosensing system of claim 15, wherein the carbohydrate is glucose, galactose, sucrose, or xylose.

17. The biosensing system of claim 1, wherein the analyte is an alcohol.

18. The biosensing system of claim 17, wherein the alcohol is ethanol, methanol, or butanol.

19. The biosensing system of claim 1, wherein the at least one oxidase comprises an enzyme from EC number 1.1.3.2, and the analyte is galactose, glucose, lactose, or sucrose.

20. The biosensing system of claim 1, wherein the at least one oxidase comprises an enzyme from EC number 1.1.3.4, and the analyte is glucose.

21. The biosensing system of claim 1, wherein the at least one oxidase comprises an enzyme from EC number 1.1.3.10, and the analyte is glucose or xylose.

22. The biosensing system of claim 1, wherein the at least one oxidase comprises an enzyme from EC number 1.1.3.13, and the analyte is ethanol, methanol, or butanol.

23. The biosensing system of claim 1, wherein the at least one oxidase is a purified enzyme.

24. The biosensing system of claim 1, wherein the input signal generated from the chemical reaction comprises oxygen, and wherein the output signal is an optical signal.

25. A method for measuring the concentration of an analyte in a solution, the method comprising:
obtaining a biosensing system having an optode comprising an optical fiber having a distal tip and a proximal tip; a photon-detection device coupled to the proximal tip; and a signal processing system coupled to the photon-detection device;
placing the distal tip into the solution, the distal tip comprising a transducer layer and a biocomponent layer, wherein the biocomponent layer comprises at least one oxidase from Enzyme Commission number 1 (EC 1) that catalyzes a chemical reaction with the analyte, wherein the transducer layer converts an input signal generated from the chemical reaction with the analyte in the biocomponent layer into an output signal detectable by the photon-detection device; and
using the signal processing system to generate a value from the output signal detectable by the photon-detection device that corresponds to the concentration of the analyte in the solution.

26. The method of claim 25, wherein the method further comprises sterilizing the distal tip of the biosensing system prior to use.

27. The method of claim 25, wherein the method further comprises using the value from the output signal to assess one or more bioprocesses of a microorganism.
